(19) United States
(12) Patent Application Publication
(10) Pub. No.: US 2023/0158102 A1
(43) Pub. Date: May 25, 2023

(54) COMPOUNDS FOR USE IN THE TREATMENT OF CORONAVIRUS INFECTION

(71) Applicant: PHARMA MAR, S.A., Madrid (ES)

(72) Inventors: José María FERNÁNDEZ SOUSA-FARO, Madrid (ES); Pablo AVILÉS MARÍN, Madrid (ES); Alejandro LOSADA GONZÁLEZ, Madrid (ES); Salvador FUDIO MUÑOZ, Madrid (ES)

(21) Appl. No.: 17/908,524

(22) PCT Filed: Mar. 2, 2021
(86) PCT No.: PCT/EP2021/055137

§ 371 Date: Aug. 31, 2022

(30) Foreign Application Priority Data
Mar. 2, 2020 (EP) 20382152.5
Mar. 13, 2020 (EP) 20382192.1
Apr. 2, 2020 (EP) 20382266.3
Apr. 27, 2020 (EP) 20382339.8
Sep. 16, 2020 (EP) 20382815.7
Sep. 16, 2020 (EP) 20382816.5

(51) Int. Cl. A61K 38/15; A61K 31/573; A61K 31/4178; A61K 31/138; A61K 31/341; A61P 31/14

(52) U.S. Cl. CPC A61K 38/15; A61K 31/573; A61K 31/4178; A61K 31/138; A61K 31/341; A61P 31/14

(57) ABSTRACT

A compound of general formula (I) wherein X, Y, n, p, q, and R₁-R₁₇ take various meanings, for use in the treatment of Coronavirus infection.

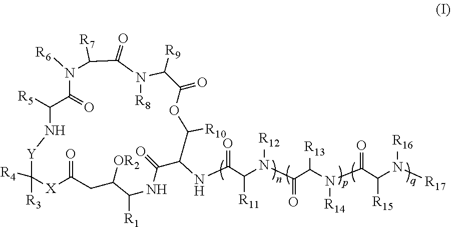

### FIELD OF THE INVENTION

The present invention relates to the treatment of Coronavirus infection.

### BACKGROUND OF THE INVENTION

Coronaviruses (CoVs) are enveloped single-stranded, positive-sense RNA viruses with genomes ranging between 26.2-31.7 kb. This large, capped and polyadenylated genome contains seven common coronavirus genes in the following conserved order: 5′-ORF1a-ORF1b-S-ORF3-E-M-N-3′. ORF1a/b produces a genome-length mRNA (mRNA1) that encodes two overlapping viral replicase proteins in the form of polyproteins 1a (pp1a) and pp1ab. These polyproteins are proteolytically processed by virally encoded proteases into mature nonstructural proteins (nsp1 to nsp16), which assemble to form a membrane-associated viral replicase-transcriptase complex (RTC). The last third of the genome produces subgenomic (sg) mRNAs that encode the four structural proteins, spike (S), envelope (E), membrane (M), and nucleocapsid (N), as well as a number of accessory proteins. CoVs belong to the subfamily Coronavirinae in the family of Coronaviridae of the order Nidovirales. The family includes four genera: α-coronavirus, β-coronavirus, γ-coronavirus and δ-coronavirus. SARS (severe acute respiratory syndrome)-CoV-2 and SARS-CoV are in the β-coronavirus genera and share around 80% of their genomes. The coronavirus N protein is abundantly produced within infected cells. N protein has multiple functions, including binding to viral RNA to form the helical ribonucleocapsid and has a structural role in coronavirus assembly. The N protein has also been proposed to have roles in virus replication, transcription and translation.

Coronaviruses (CoVs) infect a variety of human and animal hosts, causing illnesses that range from gastrointestinal tract infections, encephalitis and demyelination in animals to mostly upper relatively mild respiratory tract infections in humans. However the zoonotic coronaviruses, SARS-CoV, MERS CoV and Wuhan coronavirus (2019-nCoV, recently renamed as SARS-CoV-2) can cause severe illness and death. The disease caused by SARS-CoV-2 is called Coronavirus disease 2019 or COVID-19.

The WHO has declared the 2019-2020 coronavirus outbreak to be a Public Health Emergency of International Concerns (PHEIC). As of 12 Feb. 2021, according to the WHO, there were 107,252,265 cases of SARS-CoV-2 including 2,355,339 deaths. There is no specific treatment for a SARS-CoV infection, including SARS-Cov (which causes SARS) and SARS-CoV-2 (which causes COVID-19). A number of vaccines have been developed and, since December 2020, approved for immunisation of individuals for the prevention of COVID-19. However, due to viral mutation, vaccine take up and/or other factors, there remains high hospitalisation rates for patients having COVID-19.

As such, there is an urgent un-met medical need for a treatment for CoV infection, and in particular a treatment for COVID-19. The present invention addresses this need. In addition, there also exists a need for a treatment that does not target viral proteins and therefore is effective against variants of SARS-CoV. The present invention also addresses this need.

### SUMMARY OF THE INVENTION

In one aspect, the present invention is directed to a compound of general formula I, or a pharmaceutically acceptable salt or stereoisomer thereof,

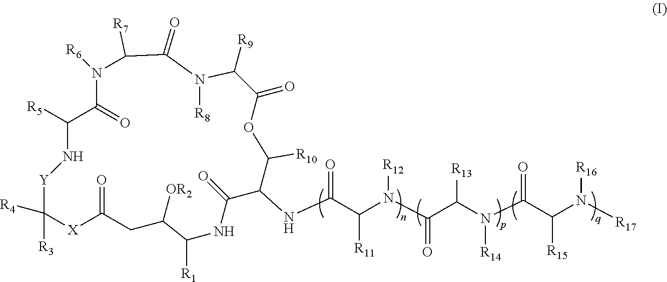

wherein X is selected from O and NH;
Y is selected from CO and —COCH(CH3)CO—;
each n and p is independently selected from 0 and 1, and q is selected from 0, 1 and 2;
each R1, R3, R5, R9, R11, and R15 is independently selected from hydrogen, substituted or unsubstituted C1-C6 alkyl, substituted or unsubstituted C2-C6 alkenyl, and substituted or unsubstituted C2-C6 alkynyl;
R2 is selected from hydrogen, CORa, COORa, substituted or unsubstituted C1-C6 alkyl, substituted or unsubstituted C2-C6 alkenyl, and substituted or unsubstituted C2-C6 alkynyl;
each R4, R8, R10, R12, and R16 is independently selected from hydrogen and substituted or unsubstituted C1-C6 alkyl;
each R7 and R13 is independently selected from hydrogen, substituted or unsubstituted C1-C6 alkyl, substituted or unsubstituted C2-C6 alkenyl, and substituted or unsubstituted C2-C6 alkynyl; each R6 and R14 is independently selected from hydrogen and substituted or unsubstituted C1-C6 alkyl; or R6 and R7 and/or R13 and R14 together with the corresponding N atom and C atom to which they are attached may form a substituted or unsubstituted heterocyclic group;
R17 is selected from hydrogen, CORa, COORa, CONHRb, COSRc, (C═NRb)ORa, (C═NRb)NHRb, (C═NRb)SRc, (C═S)ORa, (C═S)NHRb, (C═S)SRc, SO2Rc, SO3Rc, substituted or unsubstituted C1-C12 alkyl, substituted or unsubstituted C2-C12 alkenyl, substituted or unsubstituted C2-C12 alkynyl, substituted or unsubstituted aryl, and substituted or unsubstituted heterocyclic group, with the proviso that when n, p, and q are 0 then R17 is not hydrogen; and
each Ra, Rb, and Rc is independently selected from hydrogen, substituted or unsubstituted C1-C12 alkyl, substituted or unsubstituted C2-C12 alkenyl, substituted or unsubstituted C2-C12 alkynyl, substituted or unsubstituted aryl, and substituted or unsubstituted heterocyclic group;
for use in the treatment of coronavirus (CoV) infection.

In a particular aspect, the compound of general formula I is PLD, or a pharmaceutically acceptable salt or stereoisomer thereof.

In another aspect, the present invention is also directed to a pharmaceutical composition comprising a compound as defined herein, and a pharmaceutically acceptable carrier, for use in the treatment of CoV infection.

In another aspect, the present invention is directed to the use of a compound as defined herein, in the manufacture of a medicament for the treatment of CoV infection.

In another aspect, the present invention is directed to a method for treating any mammal, preferably a human, affected by a CoV infection, wherein the method comprises administering to individual in need thereof a therapeutically effective amount of a compound as defined herein.

In another aspect of the invention, there is provided a compound as defined herein, for use in the treatment of COVID-19. Particularly, the present invention provides PLD for use in the treatment of COVID-19. COVID-19 is the disease that results from infection by SARS-CoV-2.

In another aspect of the invention, there is provided a compound as defined herein, for use in the treatment of pneumonia caused by COVID-19. Particularly, the present invention provides PLD for use in the treatment of pneumonia caused by COVID-19. In another aspect of the invention, there is provided a compound as defined herein, for use in the treatment of acute respiratory distress syndrome (ARDS) caused by COVID-19. Particularly, the present invention provides PLD for use in the treatment of ARDS caused by COVID-19.

In another aspect of the invention, there is provided a compound as defined herein, for use in reducing complications associated with CoV infection, including hospitalization, ICU and death.

In another aspect of the invention, there is provided a compound as defined herein, for use in the prophylaxis, reduction or treatment of COVID persistent (also known as long COVID or post-COVID syndrome).

In another aspect of the invention, there is provided a compound as defined herein, for use in reducing the infectivity of CoV patients. The patients may be asymptomatic or not very symptomatic patients. In another aspect of the invention, there is provided a compound as defined herein, for use in reducing the occurrence of supercontagators (asymptomatic or not very symptomatic patients with high viral loads (e.g. TC<25)).

In another aspect of the invention, there is provided a compound as defined herein, for use in the treatment of coronavirus (CoV) infection (including treatment of COVID-19, treatment of pneumonia caused by COVID-19 and any of the uses as herein defined), wherein the compound is administered in combination with a corticosteroid. In a particular embodiment, there is provided PLD for use in the treatment of coronavirus (CoV) infection (including treatment of COVID-19, treatment of pneumonia caused by COVID-19 and any of the uses as herein defined), wherein the compound is administered in combination with dexamethasone. The compound and corticosteroid may be administered concurrently, separately or sequentially.

In another aspect of the invention, there is provided a corticosteroid, for use in the treatment of coronavirus (CoV) infection (including treatment of COVID-19, treatment of pneumonia caused by COVID-19 and any of the uses as herein defined), wherein the corticosteroid is administered in combination with a compound according to the present invention.

In another aspect of the invention, there is provided a compound as defined herein and a corticosteroid, for use in the treatment of coronavirus (CoV) infection (including treatment of COVID-19, treatment of pneumonia caused by COVID-19 and any of the uses as herein defined).

In another aspect, there is provided a method of treating a coronavirus (CoV) infection (including treatment of COVID-19, treatment of pneumonia caused by COVID-19 and any of the uses as herein defined), said method comprising administering a combination of a compound according to the present invention and a corticosteroid.

In another aspect, the administration regimens disclosed herein are used in the methods of treatment according to the present invention.

In another aspect, the administration regimens disclosed herein are used in the use of a compound according to the present invention in the manufacture of a medicament for the treatments as defined herein.

In another aspect, there is provided use of a compound as defined herein, in the manufacture of a medicament for the treatment of CoV infection; wherein said treatment includes administration of a corticosteroid.

In another aspect, there is provided use of a corticosteroid in the manufacture of a medicament for the treatment of CoV infection; wherein said treatment includes administration of a compound as defined herein.

In another aspect, there is provided use of a compound as defined herein and a corticosteroid in the manufacture of a medicament for the treatment of CoV infection.

In another aspect, there is provided a pharmaceutical package comprising a compound as defined herein and a corticosteroid, optionally further comprising instructions.

In another aspect, there is provided a kit comprising the compound as defined herein together with instructions for treating CoV infections. In another aspect, there is provided a kit comprising a compound as defined herein and a corticosteroid together with instructions for treating CoV infections.

The following embodiments apply to all aspects of the present invention.

R₃ and R₄ may be independently selected from hydrogen and substituted or unsubstituted C₁-C₆ alkyl. R₃ may be isopropyl and R₄ may be hydrogen. R₃ and R₄ may be methyl (this compound is also designated a compound of general formula I).

R₁₁ may be selected from hydrogen and substituted or unsubstituted C₁-C₆ alkyl. R₁₁ may be methyl or isobutyl. R₁₁ may be methyl and n=1 (this compound is also designated a compound of general formula III).

R₁, R₅, R₉, and R₁₅ may be independently selected from hydrogen and substituted or unsubstituted C₁-C₆ alkyl. R₁ may be selected from sec-butyl and isopropyl, R₅ may be isobutyl, R₉ may be p-methoxybenzyl, and R₁₅ may be selected from methyl and benzyl.

R₈, R₁₀, R₁₂, and R₁₆ may be independently selected from hydrogen and substituted or unsubstituted C₁-C₆ alkyl. R₈, R₁₀ and R₁₂ may be methyl, and R₁₆ may be hydrogen.

R₆ and R₁₄ may be independently selected from hydrogen and substituted or unsubstituted C₁-C₆ alkyl. R₆ may be selected from hydrogen and methyl, and R₁₄ may be hydrogen.

R₇ and R₁₃ may be independently selected from hydrogen and substituted or unsubstituted C₁-C₆ alkyl. R₇ may be methyl and R₁₃ may be selected from hydrogen, methyl, isopropyl, isobutyl, and 3-amino-3-oxopropyl.

R₆ and R₇ and/or R₁₃ and R₁₄ together with the corresponding N atom and C atom to which they are attached may form a substituted or unsubstituted pyrrolidine group.

R₂ may be selected from hydrogen, substituted or unsubstituted C₁-C₆ alkyl, and CORₐ, and wherein Rₐ may be a substituted or unsubstituted C₁-C₆ alkyl. R₂ may be hydrogen.

R₁₇ may be selected from hydrogen, CORₐ, COORₐ, CONHR_{b}, (C═S)NHR_{b}, and SO₂R_{c}, and wherein each Rₐ, R_{b}, and R_{c} may be independently selected from substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group. R₁₇ may be selected from hydrogen, COObenzyl, CObenzo[b]thiophen-2-yl, SO₂(p-methylphenyl), COCOCH₃ and COOC(CH₃)₃.

X may be NH. X may be O. Y may be CO. Y may be —COCH(CH₃)CO—.

The compound may be PLD, or pharmaceutically acceptable salts or stereoisomers thereof. The compound may be PLD.

The compound may be didemninB, or pharmaceutically acceptable salts or stereoisomers thereof. The compound may be didemninB.

The use may be use in the treatment of COVID-19 and/or use in the treatment of pneumonia caused by COVID-19.

The CoV infection may be mild infection; and/or moderate infection; and/or severe infection.

The CoV infection may be acute CoV infection, preferably wherein the CoV infection is acute COVID-19 infection; and/or may be ongoing symptomatic CoV infection, preferably wherein the CoV infection is ongoing symptomatic COVID-19 infection; and/or may be post-CoV syndrome, CoV persistent or long CoV; preferably wherein the CoV infection is post-COVID-19 syndrome, COVID persistent or long COVID. The post-CoV syndrome, CoV persistent or long CoV may include one or more symptoms arising from the cardiovascular, respiratory, gastrointestinal, neurological, musculoskeletal, metabolic, renal, dermatological, otolaryngological, haematological and autonomic systems; psychiatric problems, generalised pain, fatigue and/or persisting fever.

The use may be in the treatment of a patient with signs and symptoms of CoV infection (preferably COVID-19) for up to 4 weeks; and/or from 4 weeks to 12 weeks; and/or for more than 12 weeks.

The use may be in the prophylaxis, reduction or treatment of COVID persistent, long COVID or post-COVID syndrome; preferably wherein the prophylaxis, reduction or treatment minimises the likelihood that a patient suffers from COVID persistent, long COVID or post-COVID syndrome symptoms; and/or reduces the severity of such symptoms; further preferably wherein the treatment minimising the symptoms of CoV infection.

The treatment may reduce the infectivity of CoV patients; including wherein the patient is asymptomatic or not very symptomatic yet has a high viral load.

The compound may be administered in combination with a corticosteroid, preferably dexamethasone. The compound and corticosteroid may be administered concurrently, separately or sequentially.

The compound may be administered according to a regimen of a once daily dose for 10 days, 9 days, 8 days, 7 days, 6 days, 5 days, 4 days, 3 days, 2 days or 1 day; preferably 2-5 days, 3-5 days, or 3, 4 or 5 days; most preferably 3 days or 5 days; most preferably 3 days.

The compound may be administered at a dose of 5 mg a day or less, 4.5 mg a day or less, 4 mg a day or less, 3.5 mg a day or less, 3 mg a day or less, 2.5 mg a day or less or 2 mg a day or less; 0.5 mg/day, 1 mg/day, 1.5 mg/day, 2 mg/day, 2.5 mg/day, 3 mg/day, 3.5 mg/day, 4 mg/day, 4.5 mg/day, or 5 mg/day; preferably 1 mg/day, 1.5 mg/day, 2 mg/day or 2.5 mg/day; preferably 1.5-2.5 mg/day; further preferably 1.5 mg/day, 2 mg/day or 2.5 mg/day.

The compound may be administered at a total dose of 1-50 mg, 1-40 mg, 1-30 mg, 1-20 mg, 1-15 mg, 3-15 mg, 3-12 mg, 4-12 mg, 4-10 mg, or 4.5-10 mg; 4 mg, 4.5 mg, 5 mg, 5.5 mg, 6 mg, 6.5 mg, 7 mg, 7.5 mg, 8 mg, 8.5 mg, 9 mg, 9.5 mg or 10 mg; preferably 4.5 mg, 5 mg, 6 mg, 7.5 mg, 8 mg, 9 mg or 10 mg; more preferably 4.5-7.5 mg/day. The total dose may be split over 1, 2, 3, 4, 5, 6, 7, 8, 9 or 10 days, preferably 3 days or 5 days; most preferably 3 days.

The compound may be administered at a once daily dose for 3 days at a dose of 1.5-2.5 mg/day. The dose may be 1.5 mg/day. The dose may be 2.5 mg/day.

The compound may be PLD administered as a 1.5-hour infusion, once a day for 3 consecutive days. 1.5 mg of PLD may be administered as a 1.5-hour infusion, once a day for 3 consecutive days. 2 mg of PLD may be administered as a 1.5-hour infusion, once a day for 3 consecutive days. 2.5 mg of PLD may be administered as a 1.5-hour infusion, once a day for 3 consecutive days. 1 mg of PLD may be administered as a 1.5-hour infusion, once a day for 5 consecutive days. 2 mg of PLD may be administered as a 1.5-hour infusion, once a day for 5 consecutive days.

The regimen may be a single dose (1 day). The compound may be administered as a single dose of 1-10 mg, 4-10 mg, 4.5-10 mg; 4 mg, 4.5 mg, 5 mg, 5.5 mg, 6 mg, 6.5 mg, 7 mg, 7.5 mg, 8 mg, 8.5 mg, 9 mg, 9.5 mg or 10 mg; preferably 4.5 mg, 5 mg, 6 mg, 7.5 mg, 8 mg, 9 mg or 10 mg; more preferably 5-9 mg, 6.5-8.5 mg, 7-8 mg or 7.5 mg. The compound may be PLD administered as a single dose 1.5-hour infusion.

The single dose regimen may be utilised with all therapies set out in the present invention. The single dose regimen may be utilised with mild infection cases. The single dose regimen may, however, be utilised with moderate and/or severe infection cases. The combination use with corticosteroids (including subsequent corticosteroid administration) may in embodiments be used with the single dose regimen.

The multi-day regimen may be utilised with all therapies set out in the present invention. The multi-day regimen may be utilised with moderate and/or severe infection cases. The multi-day regimen may, however, also be utilised with mild infection cases.

The corticosteroid may be administered daily on the same day(s) as administering a compound according to the present invention. The corticosteroid may be administered on one or more subsequent days. The corticosteroid may be administered on 1, 2, 3, 4, 5, 6, 7, 8, 9, 10 or more subsequent days. The corticosteroid may be administered at a higher dose when administered on the same day as a compound according to the present invention and at a lower dose on subsequent day(s). The corticosteroid may be dexamethasone.

The compound according to the present invention may be administered at a dose according to the present invention on days 1-3 of the dosage regimen. The corticosteroid may be administered intravenously on days 1-3 of the dosage regimen. The corticosteroid may thereafter be administered by oral administration or IV from Day 4 and up to Day 10 (as per physician judgement according to patient clinical condition and evolution). The corticosteroid may be dexamethasone. The dose may be 6.6 mg/day IV on Days 1 to 3 (for example 8 mg dexamethasone phosphate), followed by dexamethasone 6 mg/day (for example 7.2 mg dexamethasone phosphate or 6 mg dexamethasone base) oral administration or IV from Day 4 and up to Day 10.

In embodiments, dexamethasone is dexamethasone phosphate and is, for example, administered at a dose of 8 mg/day IV on Days 1 to 3, followed by dexamethasone 7.2 mg/day oral administration or IV from Day 4 and up to Day 10.

The compound according to the present invention may be administered as an infusion, preferably a 1 hour infusion, a 1.5 hour infusion, a 2 hour infusion, a 3 hour infusion or longer; particularly preferably a 1.5 hour infusion.

The regimen may be 1.5 mg of plitidepsin administered as a 1.5-hour infusion, once a day for 3 consecutive days; or 2 mg of plitidepsin administered as a 1.5-hour infusion, once a day for 3 consecutive days; or 2.5 mg of plitidepsin administered as a 1.5-hour infusion, once a day for 3 consecutive days; or 1 mg of plitidepsin administered as a 1.5-hour infusion, once a day for 5 consecutive days; or 2 mg of plitidepsin administered as a 1.5-hour infusion, once a day for 5 consecutive days.

The regimen may be 7.5 mg of plitidepsin administered as a 1.5-hour infusion, as a single dose on day 1.

The compound according to the present invention may be administered using a loading dose and a maintenance dose.

The regimen according to the present invention may be:
a loading dose of 2.5 mg for day 1, and followed by a maintenance dose of 2 mg/day for subsequent days;
a loading dose of 2.5 mg for day 1, and followed by a maintenance dose of 1.5 mg/day for subsequent days;
a loading dose of 2.5 mg for day 1, and followed by a maintenance dose of 1 mg/day for subsequent days;
a loading dose of 2.5 mg for day 1, and followed by a maintenance dose of 0.5 mg/day for subsequent days;
a loading dose of 2 mg for day 1, and followed by a maintenance dose of 1.5 mg/day for subsequent days;
a loading dose of 2 mg for day 1, and followed by a maintenance dose of 1 mg/day for subsequent days;
a loading dose of 2 mg for day 1, and followed by a maintenance dose of 0.5 mg/day for subsequent days;
a loading dose of 1.5 mg for day 1, and followed by a maintenance dose of 1 mg/day for subsequent days;
a loading dose of 1.5 mg for day 1, and followed by a maintenance dose of 0.5 mg/day for subsequent days; or
a loading dose of 1 mg for day 1, and followed by a maintenance dose of 0.5 mg/day for subsequent days.

The compound according to the present invention may be administered in combination with a corticosteroid. The corticosteroid may be administered on the same days as administration of the compound.

The corticosteroid may also be administered on one or more subsequent days; for example wherein the corticosteroid is administered with the compound on days 1-3 and the corticosteroid is further administered on one or more of days 4-10.

The corticosteroid may be administered intravenously on days when the compound is administered but administered by oral administration or IV on subsequent days.

The corticosteroid may be dexamethasone. Dexamethasone may be administered at a dose of 6.6 mg/day IV on days when the compound is administered.

Dexamethasone may be administered at a dose of 6 mg/day oral administration or IV on subsequent days, preferably one or more of days 4, 5, 6, 7, 8, 9 and 10.

The dexamethasone dose as defined herein refers to the base weight. The dose can therefore be adjusted if used in salt form. For example, the dexamethasone may be dexamethasone phospate such that 8 mg/day is equivalent to 6.6 mg of dexamethasone base, and 7.2 mg/day is equivalent to 6 mg of dexamethasone base.

The compound according to the present invention, particularly PLD, may be administered 1.5 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).

The compound according to the present invention, particularly PLD, may be administered 2.0 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).

The compound according to the present invention, particularly PLD, may be administered 2.5 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).

The corticosteroid may be administered 20 to 30 minutes prior to starting treatment with the compound as defined herein.

In regimens according to the present invention, the patient may additionally receive the following medications, preferably 20 to 30 minutes prior to starting treatment with the compound according to the present invention:

Ondansetron 8 mg IV (or equivalent);
Diphenhydramine hydrochloride 25 mg IV (or equivalent); and
Ranitidine 50 mg IV (or equivalent).

In regimens according to the present invention, on Days 4 and 5, patients may receive ondansetron (or equivalent) 4 mg twice a day PO.

When administered as a single dose, patients may receive the following prophylactic medications 20-30 minutes prior to plitidepsin infusion:
Diphenhydramine hydrochloride 25 mg i.v;
Ranitidine 50 mg i.v;
Dexamethasone 6.6 mg intravenously;
Ondansetron 8 mg i.v. in slow infusion of 15 minutes.

Ondansetron 4 mg orally may be given every 12 hours for 3 days after plitidepsin administration to relieve drug-induced nausea and vomiting. If plitidepsin is administered in the morning the patient may receive the first dose of ondansetron in the afternoon.

### DESCRIPTION OF THE FIGURES

The invention is further described in the following non-limiting figures:

FIG. 1—Graphical representation of the antiviral activity (—♦— RLUs) and toxicity (—●— Viability) of several concentrations (μM) of compound 3 in MT-2 cells (FIG. 1A) and in preactivated PBMCs (FIG. 1B), both infected with a recombinant virus (NL4.3 Luc). Graphical representations are at least mean of two independent experiments for MT-2 cells and four for PBMCs.

FIG. 2—Graphical representation of the antiviral activity (—♦— RLUs) and toxicity (—●— Viability) of several concentrations (μM) of compound 8 in MT-2 cells (FIG. 2A) and in preactivated PBMCs (FIG. 2B), both infected with a recombinant virus (NL4.3 Luc). Graphical representations are at least mean of two independent experiments for MT-2 cells and four for PBMCs.

FIG. 3—Graphical representation of the antiviral activity (—♦— RLUs) and toxicity (—▪— Viability) of several concentrations (μM) of compound 9 in MT-2 cells (FIG. 3A) and in preactivated PBMCs (FIG. 3B), both infected with a recombinant virus (NL4.3 Luc). Graphical representations are at least mean of two independent experiments for MT-2 cells and four for PBMCs.

FIG. 4—Graphical representation of the antiviral activity (—♦— RLUs) and toxicity (—▪— Viability) of several concentrations (μM) of compound 10 in MT-2 cells (FIG. 4A) and in preactivated PBMCs (FIG. 4B), both infected with a recombinant virus (NL4.3 Luc). Graphical representations are at least mean of two independent experiments for MT-2 cells and four for PBMCs.

FIG. 5—Graphical representation of the antiviral activity (—♦— RLUs) and toxicity (—▪— Viability) of several concentrations (μM) of compound 11 in MT-2 cells (FIG. 5A) and in preactivated PBMCs (FIG. 5B), both infected with a recombinant virus (NL4.3 Luc). Graphical representations are at least mean of two independent experiments for MT-2 cells and four for PBMCs.

FIGS. 6-10 show fluorescence images showing a) cell growth and b) antiviral activity for DMSO 24 hpi against HCoV-229E infected Huh-7 cells (A1, A2, A3, A4, A5 from Table 1). It can be seen that cells remain viable but that no antiviral effect is seen.

FIGS. 11-14 show fluorescence images showing a) cell growth and b) antiviral activity for Compound 240 (DidemninB) 24 hpi against HCoV-229E infected Huh-7 cells at 50 nM, 5 nM and 0.5 nM concentrations (B1, B2, B3, B4 from Table 1 respectively). It can be seen that cells remain viable at all concentrations, including high concentrations; and that remarkable antiviral properties are seen across all concentrations, even at sub nano-molar concentrations.

FIGS. 15-18 show fluorescence images showing a) cell growth and b) antiviral activity for PLD 24 hpi against HCoV-229E infected Huh-7 cells at 50 nM, 5 nM and 0.5 nM concentrations (C1, C2, C3, C4 from Table 1 respectively). Again, it can be seen that cells remain viable at all concentrations, including high concentrations; and that remarkable antiviral properties are seen across all concentrations, even at sub nano-molar concentrations.

FIGS. 19-21 show fluorescence images showing a) cell growth and b) antiviral activity for Compound 9 24 hpi against HCoV-229E infected Huh-7 cells at 50 nM, 5 nM and 0.5 nM concentrations (D1, D2, D3, from Table 1 respectively). Again, it can be seen that cells remain viable at all concentrations, including high concentrations; and that remarkable antiviral properties are seen across all concentrations, even at sub nano-molar concentrations.

FIGS. 22-24 show fluorescence images showing a) cell growth and b) antiviral activity for Compound 10 24 hpi against HCoV-229E infected Huh-7 cells at 50 nM, 5 nM and 0.5 nM concentrations (E1, E2, E3, from Table 1 respectively). Again, it can be seen that cells remain viable at all concentrations, including high concentrations; and that remarkable antiviral properties are seen across all concentrations, even at sub nano-molar concentrations.

FIGS. 25-28 show fluorescence images showing a) cell growth and b) antiviral activity for PLD (second run) 24 hpi against HCoV-229E infected Huh-7 cells at 50 nM, 5 nM and 0.5 nM concentrations (F1, F2, F3, F4 from Table 1 respectively). Again, it can be seen that cells remain viable at all concentrations, including high concentrations; and that remarkable antiviral properties are seen across all concentrations, even at sub nano-molar concentrations.

FIGS. 29 and 30 show total plasma concentration profiles vs. time predicted for dosing schedules and administration according to the present invention.

FIG. 31 shows total plasma concentration profiles vs. time predicted for further dosing schedules and administration according to the present invention.

FIG. 32 shows dose response curves showing the antiviral effect of plitidepsin on SARS-CoV-2 in vero cells.

FIG. 33 shows dose response curves showing the antiviral effect of plitidepsin on SARS-CoV-2 in vero cells.

FIG. 34 shows x-rays showing the effects of PLD administration on a patient with bilateral pneumonia.

FIG. 35 shows x-rays showing the effects of PLD administration on a patient with unilateral pneumonia.

FIG. 36 shows C-reactive protein tests for patients treated with PLD.

FIG. 37 shows the viral load log of Patient 4 (FIG. 37a), Patient 5 (FIG. 37b), Patient 6 (FIG. 37c) and Patient 7 (FIG. 37d). Patients were administered PLD as a 90 minute IV infusion daily for 3 consecutive days (day 1-3) with viral load assessed by PCR at baseline, day 4, day 7, day 15 and day 31.

FIG. 38 shows total vs. plasma concentration profiles for single dose plitidepsin 7.5 mg and 1.5, 2.0 and 2.5 mg on day 1 to 3, using a 1.5 hour infusion.

### DETAILED DESCRIPTION

The present invention will now be further described. In the following passages, different aspects of the invention are defined in more detail. Each aspect so defined may be combined with any other aspect or aspects or embodiment or embodiments unless clearly indicated to the contrary. In particular, any feature indicated as being preferred or advantageous may be combined with any other feature or features indicated as being preferred or advantageous.

In the present application, a number of general terms and phrases are used, which should be interpreted as follows.

The term “treating”, as used herein, unless otherwise indicated, means reversing, attenuating, alleviating or inhibiting the progress of the disease or condition to which such term applies, or one or more symptoms of such disorder or condition. The term treating as used herein may also include prophylactic treatment, that is treatment designed to prevent a disease from occurring or minimize the likelihood of a disease occurring.

“Treat”, “treating”, and “treatment” in the context of a viral infection may refer to one or more of the following: 1) reduction in the number of infected cells; 2) reduction in the number of virions present in the serum, including reduction in viral titre (which can be measured by qPCR); 3) inhibition (i.e., slowing to some extent, preferably stopping) the rate of viral replication; 4) reduction in the viral RNA load; 5) reduction in the viral infectivity titre (the number of virus particles capable of invading a host cell); and 6) relieving or reducing to some extent one or more of the symptoms associated with the viral infection. This may include inflammation associated with viral infection.

The treatment may be treating CoV infection. The treatment may be treating SARS-CoV-2 infection. The treatment may be treating COVID-19 infection. The treatment may be the treatment of COVID-19. The treatment may be the treatment of a disease that results from infection by CoV. The treatment may be the treatment of a disease that results from infection by SARS-CoV-2. The treatment may be the treatment of pneumonia caused by infection by CoV. The treatment may be the treatment of pneumonia caused by infection by SARS-CoV-2. The treatment may be the treatment of pneumonia caused by infection by COVID-19. The treatment may be the treatment of pneumonia caused by COVID-19. Similarly, the treatment may be the treatment of acute respiratory distress syndrome (ARDS) caused by infection by SARS-CoV-2 or COVID-19.

The infection may be moderate infection. The infection may be severe infection. The infection may be mild infection.

The treatment may be reducing complications associated with CoV infection, including hospitalization, ICU and death.

The present invention may be useful to treat acute COVID-19 infection (signs and symptoms of COVID-19 for up to 4 weeks); treat (or miniminse) ongoing symptomatic COVID-19 (signs and symptoms of COVID-19 from 4 weeks up to 12 weeks); or treat or minimise post-COVID-19 syndrome (signs and symptoms that develop during or following an infection consistent with COVID-19, continue for more than 12 weeks and are not explained by an alternative diagnosis.

It usually presents with clusters of symptoms, often overlapping, which can fluctuate and change over time and can affect any system in the body. Post-COVID-19 syndrome may be considered before 12 weeks while the possibility of an alternative underlying disease is also being assessed). The compounds of the present invention may treat a patient with signs and symptoms of COVID-19 for up to 4 weeks. The compounds of the present invention may treat a patient with signs and symptoms of COVID-19 from 4 weeks to 12 weeks. The compounds of the present invention may treat a patient with signs and symptoms of COVID-19 for more than 12 weeks.

The treatment may be prophylaxis, reduction or treatment of COVID persistent (also known as long COVID or post-COVID syndrome). The compounds according to the present invention can minimise the likelihood of a patient suffering from COVID persistent symptoms. The compounds according to the present invention may alternatively reduce the severity of such symptoms, preferably may minimise the symptoms of CoV infection.

Post-COVID syndrome may be considered as signs and symptoms that develop during or following an infection consistent with COVID-19 which continue for more than 12 weeks and are not explained by an alternative diagnosis. The condition usually presents with clusters of symptoms, often overlapping, which may change over time and can affect any system within the body. Many people with post-COVID syndrome can also experience generalised pain, fatigue, persisting high temperature and psychiatric problems. Symptoms include (but are not limited to) symptoms arising in the cardiovascular, respiratory, gastrointestinal, neurological, musculoskeletal, metabolic, renal, dermatological, otolaryngological, haematological and autonomic systems, in addition to psychiatric problems, generalised pain, fatigue and persisting fever.

The treatment may be reducing the infectivity of CoV patients. The present invention achieves a rapid and significant reduction in the viral burden. Reducing the viral burden may reduce the infectiveness of patients. This is particular beneficial with patients who are asymptomatic or not very symptomatic patients yet have a high viral loads (e.g. TC<25). Such patients may be supercontagators or superspreaders. Administration of compounds according to the present invention upon detection of infection can reduce the viral burden and therefore reduce the infectiveness of the patient.

The treatment may result in a reduction of viral load. This may be expressed as a replication cycle threshold (Ct) value greater than 30 (Ct>30), on day 6 after the administration. The treatment may reduce viral load from baseline. This may be expressed as a reduction in the percentage of patients requiring hospitalisation following administration. This may be expressed as a reduction in the percentage of patients requiring invasive mechanical ventilation and/or admission to the ICU following administration. This may be expressed as a reduction of patients who develop sequelae related to persistent disease. This may be expressed as an increase in the percentage of patients with normalization of analytical parameters chosen as poor prognosis criteria (including, for example, lymphopenia, LDH, D-dimer or PCR). This may be expressed as an increase in the percentage of patients with normalization of clinical criteria (disappearance of symptoms), including, for example: headache, fever, cough, fatigue, dyspnea (shortness of breath), arthromyalgia or diarrhoea.

“Patient” includes humans, non-human mammals (e.g., dogs, cats, rabbits, cattle, horses, sheep, goats, swine, deer, and the like) and non-mammals (e.g., birds, and the like). The patient my require hospitalisation for management of infection.

Plitidepsin (PLD) is a cyclic depsipeptide originally isolated from the marine tunicate Aplidium albicans. PLD is also known as Aplidin. PLD analogues are those analogues as defined herein as compounds of Formula I, II or III. In a preferred embodiment, the present invention relates to the use of PLD.

Human/eukaryotic translation elongation factor eEF1A, is a subunit of the eukaryotic translation elongation 1 complex (eEF1). This complex delivers aminoacylated tRNA to the elongating ribosomes during protein synthesis. However, eEF1A in not only a major translation factor, but also one of the most important multifunctional proteins, having roles in the quality surveillance of newly synthesised proteins, in ubiquitin-dependent degradation and in facilitating apoptosis.

The N protein of CoVs, such as SARS-CoV and TGEV (transmissible gastroenteritis coronavirus), have been shown to bind directly to eukaryotic elongation factor 1A (eEF1A). Furthermore, knockdown of eEF1A has been shown to lead to a significant reduction in virus number demonstrating that the interaction of the N protein with eEF1A is essential for viral replication.

PLD has been shown to bind to the human translation elongation factor eEF1A with a high-affinity and a low rate of dissociation. FLIM-phasor FRET experiments demonstrate that PLD localises in tumor cells sufficiently close to eEF1A as to suggest the formation of drug-protein complexes in living cells. PLD-resistant cell lines also show reduced levels of eEF1A protein and ectopic expression of eEF1A in these resistant cells restores the sensitivity to PLD, demonstrating that eEF1A is directly involved in the mechanism of action of PLD.

As explained above, the N protein of CoVs also bind to eEF1A, and this binding is essential for viral replication. Furthermore, the N protein is highly conserved within CoVs—and in particular, SARS-CoV-2 shares around 90% amino acid identity with the N-protein in SARS-CoV. However, administration and subsequent binding of PLD to eEF1A prohibits the binding of the CoV N-protein to eEF1A. This in turn prevents virus replication. The interaction between PLD and eEF1A therefore reduces the efficiency of de novo viral capsid synthesis and consequently causes a decrease in viral load.

In addition to the above, PLD binding to eEF1A prevents eEF1A from interacting with its usual binding partners. One such binding-partner is the dsRNA-activated protein kinase (PKR or EIF2AK2). Binding of PLD to eEF1A releases PKR from a complex with eEF1A leading to the activation of PKR. PKR is a known activator of the innate immune response and a key player in anti-viral immune responses. Specifically,
(i) activated PKR phosphorylates the alpha subunit of initiation factor eIF2, leading to the formation of an inactive eiF2 complex;
(ii) activated PKR induces the degradation of IκB, nuclear translocation of NF-κB and activation of the NF-κB pathway. NF-κB is a major transcription factor that regulates the genes responsible for both innate and adaptive immune responses, such as genes involved in T-cell development, maturation and proliferation;
(iii) activation of PKR induces apoptosis through a mechanism involving Fas clustering and NF-κB translocation leading to the elimination of infected cells.

Of note, protein 4a of CoVs potently suppresses the activation of PKR through the sequestration of dsRNA. PLD bypasses this viral response, leading to activation of PKR by releasing PKR from the eEF1A complex, as can be seen from the activation of PKR in the absence of viral infection.

Finally—and in addition to the above, binding of PLD to eEF1A also activates the ER-stress induced unfolded protein response (UPR), which in turn leads to a number of anti-viral responses, including again the phosphorylation of eIF2α.

Through a combination of these mechanisms—(i) inhibition of the CoV N-protein/eEF1A interaction; (ii) activation of PKR and (iii) activation of the UPR; PLD prevents CoV replication And causes the activation of host responses that lead to the elimination of CoV. Both of which contribute to an effective viral therapy. An additional advantage of targeting eEF1A is that it is a human target and as such will not mutate to evade PLD the way viral proteins do.

Accordingly, compounds of the present invention (including PLD) can be used in the treatment of CoV infection.

In these compounds the groups can be selected in accordance with the following guidance:

Alkyl groups may be branched or unbranched, and preferably have from 1 to about 12 carbon atoms. One more preferred class of alkyl groups has from 1 to about 6 carbon atoms. Even more preferred are alkyl groups having 1, 2, 3 or 4 carbon atoms. Methyl, ethyl, n-propyl, isopropyl and butyl, including n-butyl, tert-butyl, sec-butyl and isobutyl are particularly preferred alkyl groups in the compounds of the present invention. As used herein, the term alkyl, unless otherwise stated, refers to both cyclic and noncyclic groups, although cyclic groups will comprise at least three carbon ring members.

Preferred alkenyl and alkynyl groups in the compounds of the present invention may be branched or unbranched, have one or more unsaturated linkages and from 2 to about 12 carbon atoms. One more preferred class of alkenyl and alkynyl groups has from 2 to about 6 carbon atoms. Even more preferred are alkenyl and alkynyl groups having 2, 3 or 4 carbon atoms. The terms alkenyl and alkynyl as used herein, unless otherwise stated, refer to both cyclic and noncyclic groups, although cyclic groups will comprise at least three carbon ring members.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Preferably aryl groups contain from 6 to about 10 carbon ring atoms. Specially preferred aryl groups include substituted or unsubstituted phenyl, substituted or unsubstituted naphthyl, substituted or unsubstituted biphenyl, substituted or unsubstituted phenanthryl, and substituted or unsubstituted anthryl.

Suitable heterocyclic groups include heteroaromatic and heteroalicyclic groups containing from 1 to 3 separated or fused rings and from 5 to about 18 ring atoms. Preferably heteroaromatic and heteroalicyclic groups contain from 5 to about 10 ring atoms, most preferably 5, 6 or 7 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl including pyrazol-3-yl, pyrazol-4-yl and pyrazol-5-yl, pyrimidinyl, furanyl including furan-2-yl, furan-3-yl, furan-4-yl and furan-5-yl, pyrrolyl, thienyl, thiazolyl including thiazol-2-yl, thiazol-4-yl and thiazol-5-yl, isothiazolyl, thiadiazolyl including thiadiazol-4-yl and thiadiazol-5-yl, triazolyl, tetrazolyl, isoxazolyl including isoxazol-3-yl, isoxazol-4-yl and isoxazol-5-yl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl including benzo[b]thiophen-2-yl and benzo[b]thiophen-3-yl, benzothiazolyl, benzoxazolyl, imidazo[1,2-a]pyridinyl including imidazo[1,2-a]pyridine-2-yl and imidazo[1,2-a]pyridine-3-yl, quinazolinyl, quinoxalinyl, naphthyridinyl and furopyridyl. Suitable heteroalicyclic groups in the compounds of the present invention contain one, two or three heteroatoms selected from N, O or S atoms and include, e.g., pyrrolidinyl, tetrahydrofuranyl, dihydrofuranyl, tetrahydrothienyl, tetrahydrothiopyranyl, piperidinyl including piperidin-3-yl, piperidin-4-yl and piperidin-5-yl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridyl, 2-pyrrolinyl, 3-pyrrolinyl, dihydropyrrolyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

In the above mentioned groups one or more hydrogen atoms may be substituted by one or more suitable groups such as OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂. NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. When a substituent group terminates with a double bound (such as ═O and ═N—R′) it replaces 2 hydrogen atoms in the same carbon atom.

Suitable halogen substituents in the compounds of the present invention include F, Cl, Br and I.

The term “pharmaceutically acceptable salts” refers to any salt which, upon administration to the patient is capable of providing (directly or indirectly) a compound as described herein. It will be appreciated that non-pharmaceutically acceptable salts also fall within the scope of the invention since those may be useful in the preparation of pharmaceutically acceptable salts. The preparation of salts can be carried out by methods known in the art. For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts.

The compounds of the invention may be in crystalline form either as free compounds or as solvates (e.g. hydrates, alcoholates, particularly methanolates) and it is intended that both forms are within the scope of the present invention. Methods of solvation are generally known within the art. The compounds of the invention may present different polymorphic forms, and it is intended that the invention encompasses all such forms

Any compound referred to herein is intended to represent such specific compound as well as certain variations or forms. In particular, compounds referred to herein may have asymmetric centres and therefore exist in different enantiomeric or diastereomeric forms. Thus any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same or different than the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

In compounds of general formula I and II, particularly preferred R₁, R₅, R₉, R₁₁, and R₁₅ are independently selected from hydrogen and substituted or unsubstituted C₁-C₆ alkyl. More preferred R₁, R₅, R₉, R₁₁, and R₁₅ are independently selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, methyl, n-propyl, isopropyl, isobutyl, sec-butyl, 4-aminobutyl, 3-amino-3-oxopropyl, benzyl, p-methoxybenzyl, p-hydroxybenzyl, and cyclohexylmethyl are the most preferred R₁, R₅, R₉, R₁₁, and R₁₅ groups. Specifically, particularly preferred R₁ is selected from sec-butyl and isopropyl, being sec-butyl the most preferred. Particularly preferred R₅ is selected from isobutyl and 4-aminobutyl, being isobutyl the most preferred. Particularly preferred Rn is methyl and isobutyl. Particularly preferred R₉ is selected from p-methoxybenzyl, p-hydroxybenzyl, and cyclohexylmethyl, being p-methoxybenzyl the most preferred. Particularly preferred R₁₅ is selected from methyl, n-propyl, and benzyl, being methyl and benzyl the most preferred.

In compounds of general formula III, particularly preferred R₁, R₅, R₉, and R₁₅ are independently selected from hydrogen and substituted or unsubstituted C₁-C₆ alkyl. More preferred R₁, R₅, R₉, and R₁₅ are independently selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, methyl, n-propyl, isopropyl, isobutyl, sec-butyl, 4-aminobutyl, 3-amino-3-oxopropyl, benzyl, p-methoxybenzyl, p-hydroxybenzyl, and cyclohexylmethyl are the most preferred R₁, R₅, R₉, and R₁₅ groups. Specifically, particularly preferred R₁ is selected from sec-butyl and isopropyl, being sec-butyl the most preferred. Particularly preferred R₅ is selected from isobutyl and 4-aminobutyl, being isobutyl the most preferred. Particularly preferred R₉ is selected from p-methoxybenzyl, p-hydroxybenzyl, and cyclohexylmethyl, being p-methoxybenzyl the most preferred. Particularly preferred R₁₅ is selected from methyl, n-propyl, and benzyl, being methyl and benzyl the most preferred.

In compounds of general formula I, II and III, particularly preferred R₈, R₁₀, R₁₂, and R₁₆ are independently selected from hydrogen and substituted or unsubstituted C₁-C₆ alkyl. More preferred R₈, R₁₀, R₁₂, and R₁₆ are independently selected from hydrogen, methyl, ethyl, n-propyl, isopropyl and butyl, including n-butyl, tert-butyl, isobutyl and sec-butyl, and even more preferred they are independently selected from hydrogen and methyl. Specifically, particularly preferred R₈, R₁₀ and R₁₂ are methyl, and particularly preferred R₁₆ is hydrogen.

In compounds of general formula I and II, particularly preferred R₃ and R₄ are independently selected from hydrogen and substituted or unsubstituted C₁-C₆ alkyl. More preferred R₃ and R₄ are independently selected from hydrogen, substituted or unsubstituted methyl, substituted or unsubstituted ethyl, substituted or unsubstituted n-propyl, substituted or unsubstituted isopropyl, and substituted or unsubstituted butyl, including substituted or unsubstituted n-butyl, substituted or unsubstituted tert-butyl, substituted or unsubstituted isobutyl and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, methyl, isopropyl, and sec-butyl are the most preferred R₃ and R₄ groups. Specifically, particularly preferred R₃ is selected from methyl and isopropyl and particularly preferred R₄ is methyl or hydrogen.

In one embodiment of compounds of general formula I, II and III, particularly preferred R₆ and R₇ are independently selected from hydrogen and substituted or unsubstituted C₁-C₆ alkyl. More preferred R₇ is selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. More preferred R₆ is selected from hydrogen, methyl, ethyl, n-propyl, isopropyl and butyl, including n-butyl, tert-butyl, isobutyl and sec-butyl. Most preferred R₆ is selected from hydrogen and methyl and most preferred R₇ is methyl.

In another embodiment of compounds of general formula I, II and III, it is particularly preferred that R₆ and R₇ together with the corresponding N atom and C atom to which they are attached form a substituted or unsubstituted heterocyclic group. In this regard, preferred heterocyclic group is a heteroalicyclic group containing one, two or three heteroatoms selected from N, O or S atoms, most preferably one N atom, and having from 5 to about 10 ring atoms, most preferably 5, 6 or 7 ring atoms. A pyrrolidine group is the most preferred.

In one embodiment of compounds of general formula I, II and III, particularly preferred R₁₃ and R₁₄ are independently selected from hydrogen and substituted or unsubstituted C₁-C₆ alkyl. More preferred R₁₃ is selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. More preferred R₁₄ is selected from hydrogen, methyl, ethyl, n-propyl, isopropyl and butyl, including n-butyl, tert-butyl, isobutyl and sec-butyl. Most preferred R₁₃ is selected from hydrogen, methyl, isopropyl, isobutyl, and 3-amino-3-oxopropyl and most preferred R₁₄ is hydrogen.

In another embodiment of compounds of general formula I, II and III, it is particularly preferred that R₁₃ and R₁₄ together with the corresponding N atom and C atom to which they are attached form a substituted or unsubstituted heterocyclic group. In this regard, preferred heterocyclic group is a heteroalicyclic group containing one, two or three heteroatoms selected from N, O or S atoms, most preferably one N atom, and having from 5 to about 10 ring atoms, most preferably 5, 6 or 7 ring atoms. A pyrrolidine group is the most preferred.

In compounds of general formula I, II and III, particularly preferred R₂ is selected from hydrogen, substituted or unsubstituted C₁-C₆ alkyl, and CORₐ, wherein Rₐ is a substituted or unsubstituted C₁-C₆ alkyl, and even more preferred Rₐ is methyl, ethyl, n-propyl, isopropyl and butyl, including n-butyl, tert-butyl, sec-butyl and isobutyl. More preferably R₂ is hydrogen.

In compounds of general formula I, II and III, particularly preferred R₁₇ is selected from hydrogen, CORₐ, COORₐ, CONHR_{b}, (C═S)NHR_{b}, and SO₂R_{c}, wherein each Rₐ, R_{b}, and R_{c} is preferably and independently selected from substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, CORₐ, COORₐ, and SO₂Re are the most preferred R₁₇ groups, and hydrogen, COObenzyl, CObenzo[b]thiophen-2-yl, SO₂(p-methylphenyl), COCOCH₃ and COOC(CH₃)₃ are even most preferred.

In another embodiment of compounds of general formula I, II and III, it is particularly preferred that Y is CO. In another embodiment, it is particularly preferred that Y is —COCH(CH₃)CO—.

In another embodiment of compounds of general formula I, II and III, it is particularly preferred that X is O. In another embodiment, it is particularly preferred that X is NH.

In another embodiment of compounds of general formula I and H, it is particularly preferred that n, p and q are 0. In another embodiment, it is particularly preferred that n is 1 and p and q are 0. In another embodiment, it is particularly preferred that n and p are 1 and q is 0. In another embodiment, it is particularly preferred that n, p, and q are 1. In another embodiment, it is particularly preferred that n and p are 1 and q is 2.

In another embodiment of compounds of general formula III, it is particularly preferred that p and q are 0. In another embodiment, it is particularly preferred that p is 1 and q is 0. In another embodiment, it is particularly preferred that p and q are 1. In another embodiment, it is particularly preferred that p is 1 and q is 2.

In additional preferred embodiments, the preferences described above for the different substituents are combined. The present invention is also directed to such combinations of preferred substitutions of formula I, II and III above.

In the present description and definitions, when there are several groups Rₐ, R_{b}, and R_{c} present in the compounds of the invention, and unless it is stated explicitly so, it should be understood that they can be each independently different within the given definition, i.e. Rₐ does not represent necessarily the same group simultaneously in a given compound of the invention.

In compounds of general formula I, II and III when q takes a value of 2 there are two groups R₁₅ and two groups R₁₆ in the compound. It is hereby clarified that each R₁₅ and each R₁₆ group in a given compound may be independently selected among the different possibilities described above for such groups.

A particularly preferred stereochemistry for compounds of general formula I is

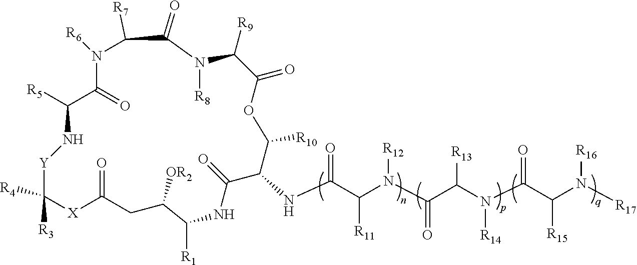

wherein X, Y, n, p, q, and R₁-R₁₇ are as defined above, and when Y is —COCH(CH₃)CO—it has the following stereochemistry:

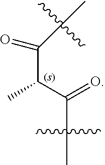

A particularly preferred stereochemistry for compounds of general formula II is

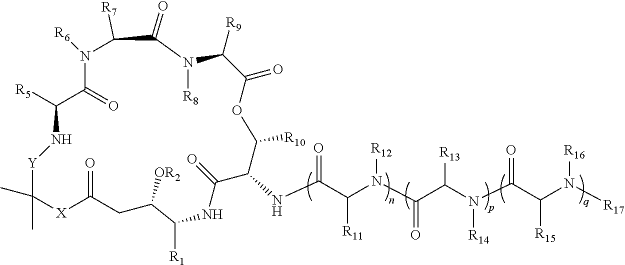

wherein X, Y, n, p, q, R₁, R₂, and R₅-R₁₇ are as defined above, and when Y is —COCH(CH₃)CO—it has the following stereochemistry:

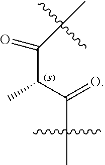

A particularly preferred stereochemistry for compounds of general formula III is

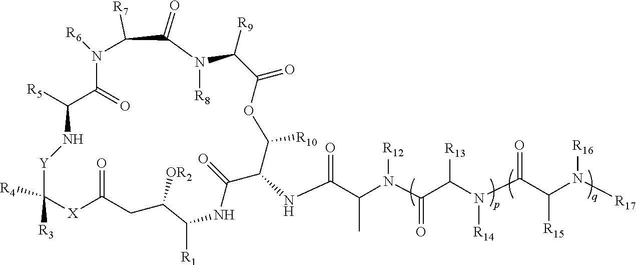

wherein X, Y, p, q, R₁-R₁₀, and R₁₂-R₁₇ are as defined above, and when Y is —COCH(CH₃)CO—it has the following stereochemistry:

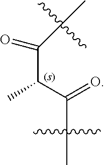

Particularly preferred compounds of the invention are the following:

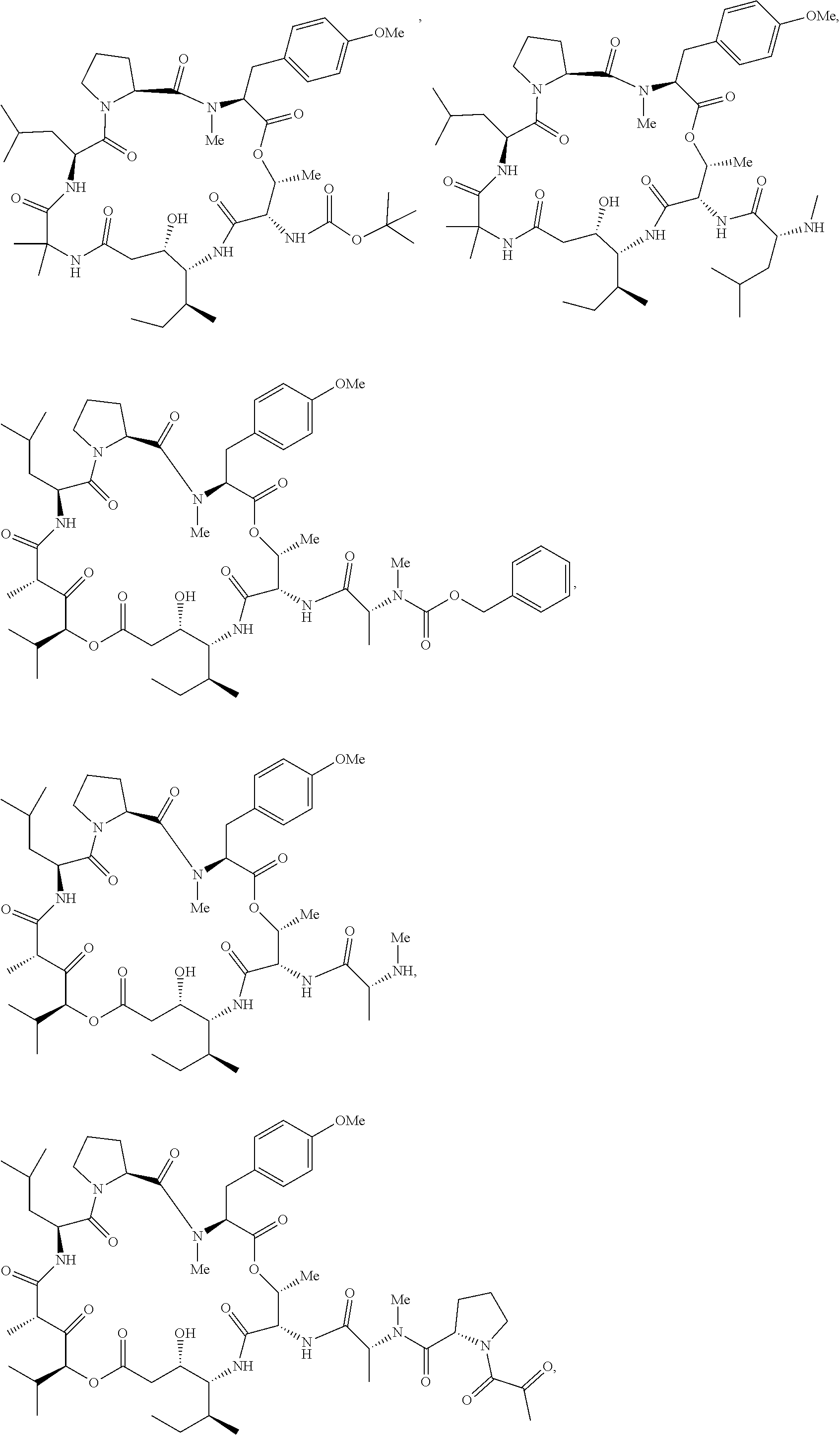

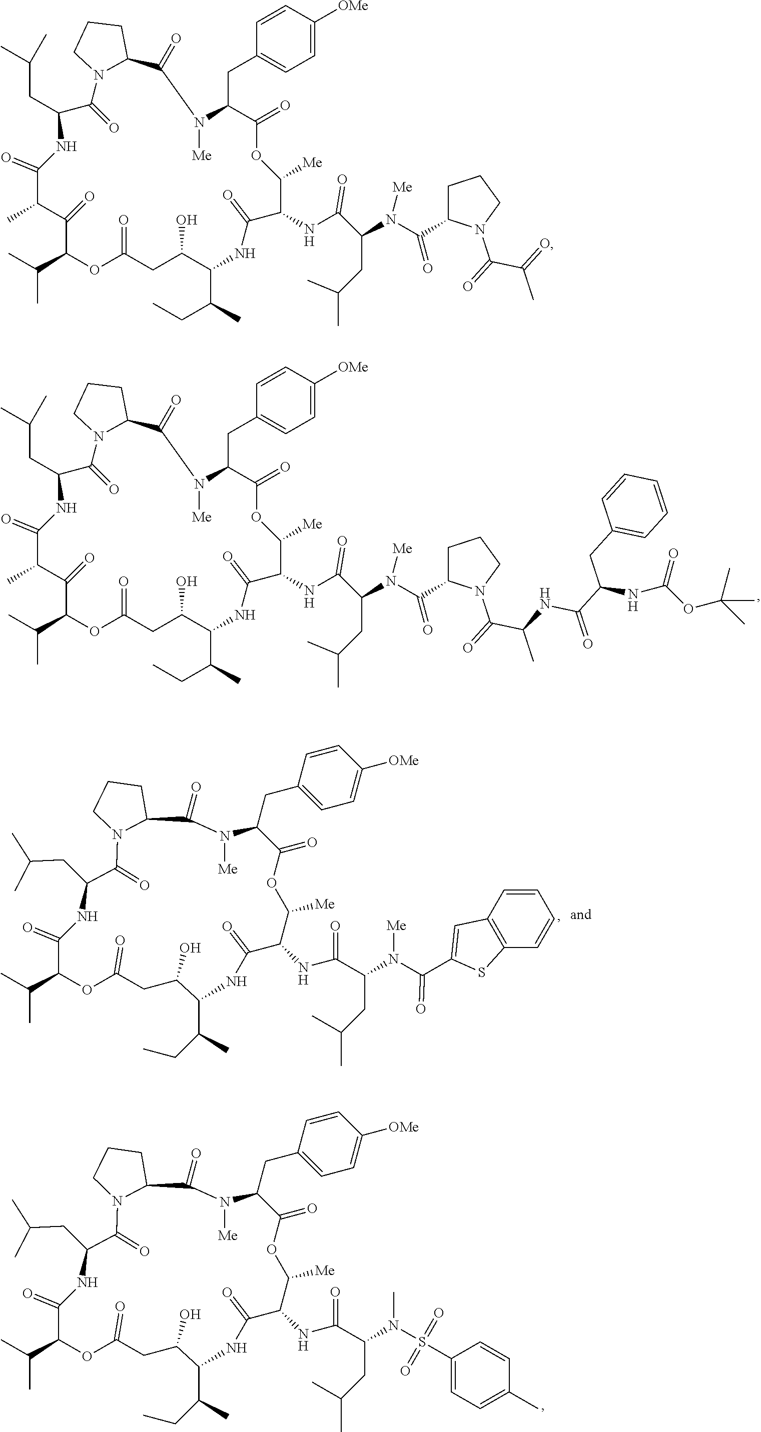

or pharmaceutically acceptable salts or stereoisomers thereof.

The compounds of general formula I, II and III may be prepared following any of the synthetic processes disclosed in Vera et al. Med. Res. Rev. 2002, 22(2), 102-145, WO 2011/020913 (see in particular Examples 1-5), WO 02/02596, WO 01/76616, and WO 2004/084812, which are incorporated herein by reference.

The preferred compound is PLD or pharmaceutically acceptable salts or stereoisomers thereof. Most preferred is PLD.

The chemical name of plitidepsin is (−)-(3S,6R,7S,10R,11S,15S,17S,20S,25aS)-11-hydroxy-3-(4-methoxybenzyl)-2,6,17-trimethyl-15-(1-methylethyl)-7-[[(2R)-4-methyl-2-[methyl[[(2S)-1-(2-oxopropanoyl)pyrrolidin-2-yl]carbonyl]amino]pentanoyl]amino]-10-[(1S)-1-methylpropyl]-20-(2-methylpropyl)tetradecahydro-15H-pyrrolo[2,1-f]-[1,15,4,7,10,20]dioxatetrazacyclotricosine-1,4,8,13,16,18,21(17H)-heptone corresponding to the molecular formula C₅₇H₈₇N₇O₁₅. It has a relative molecular mass of 1110.34 g/mol and the following structure:

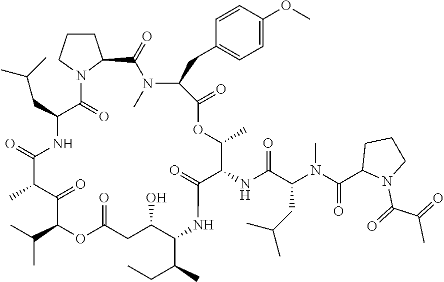 [SMILES: CC[C@H](C)[C@H]1NC(=O)[C@@H](NC(=O)[C@@H](CC(C)C)N(C)C(=O)C2CCCN2C(=O)C(C)=O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O]

Plitidepsin is a cyclic depsipeptide originally isolated from a Mediterranean marine tunicate (Aplidium albicans) and currently manufactured by full chemical synthesis. It is licensed and marketed in Australia under the brand name plitidepsin for the treatment of multiple myeloma.

In eukaryotic cells, plitidepsin has been shown to target the eukaryotic elongation factor (eEF1A), which has a key role in modulating interaction with other proteins, some of which are believed to be essential in viral replication. It is noteworthy that one of the aforementioned proteins is the coronavirus N protein, which is produced abundantly within infected cells and is known to interact with elongation factor EF1A. As said above, the interaction between plitidepsin and EF1A could therefore reduce the efficacy of de novo viral capsid synthesis and consequently lead to a decrease in viral load.

The present invention provides the use of a compound of the present invention in the treatment of CoV infection. Particularly, the present invention provides the use of PLD in the treatment of CoV infection. The term “CoV” infection means any infection from a virus in the family Coronaviridae and the sub-family Orthocoronavirinae. In one embodiment, the infection is from a virus in the genus Betacoronavirus, which includes Betacoronavirus 1, Human coronavirus HKU1, Murine coronavirus, Pipistrellus bat coronavirus HKU5, Rousettus bat coronavirus HKU9, Severe acute respiratory syndrome-related coronavirus (SARS-CoV), Tylonycteris bat coronavirus HKU4, Middle East respiratory syndrome-related coronavirus, Human coronavirus OC43 and Hedgehog coronavirus 1 (EriCoV). Preferably, the virus is SARS-CoV, or SARS-CoV-2, and most preferably SARS-CoV-2. SARS-CoV-2 was previously called 2019-nCoV and such terms may be used interchangeably herein.

In particular embodiments, the virus is SARS-CoV-2 and the associated COVID-19 disease. Mortality associated with COVID-19 disease appears to be associated with a) severe respiratory failure secondary to respiratory distress and b) an inflammatory status caused by a cytokine storm. Thus, the proportion of patients with severe disease requiring hospitalisation with or without high-flow oxygen supplements and patients requiring mechanical ventilatory support was estimated to be close to 15% and 5%, respectively, in the initial series from China. However, in Europe, the figures reported by the health authorities are higher, reaching 30% of serious cases requiring hospitalisation (in the city of Madrid) without the need for mechanical ventilation and close to 10% of patients requiring mechanical ventilation. Likewise, the duration of the need for mechanical ventilation in the Chinese series is much shorter than that reported in cities such as Madrid, so the usual flow of patients to intensive care units is being altered by the prolonged stay of patients. This is putting an enormous burden on hospital services, which has made it necessary to take extraordinary, unprecedented measures. It is believed that the magnitude of the complications initially described can be avoided or reduced through the use of the present invention in patients with early-stage COVID-19 pneumonia, since once the cytokine storm and respiratory distress take place, it is typically harder for an antiviral drug to have a beneficial therapeutic effect. However, in embodiments, the compounds of the present invention are also useful at a later stage of the viral infection, for example in patients where cytokine storm and respiratory distress have taken place.

As mentioned above, in eukaryotic cells, FLIM-FRET experiments demonstrated that plitidepsin localises sufficiently close to eEF1A to suggest the formation of drug-protein complexes. A separate set of experiments carried out with 14C-plitidepsin and eEF1A purified from rabbit muscle showed that plitidepsin binds eEF1A with high affinity and a low rate of dissociation.

Plitidepsin Activity on SARS-CoV-2 In Vitro

Several in vitro experiments aimed at determining the effect of plitidepsin on SARS-CoV-2 were carried out and are disclosed herein. Two studies, each using Vero E6 cells infected with SARS-CoV-2 and direct quantitation of SARS-CoV-2 nucleocapsid (N) protein, which is clearly involved in the mechanism of plitidepsin-induced antiviral activity, showed that plitidepsin is a potent inhibitor of SARS-CoV-2 growth in vitro, with IC₅₀ of 0.7 to 3.0 nM. In another study, human stem cell-derived pneumocyte like cells were prophylactically exposed to 10 nm plitidepsin for 1 hour and then infected with SARS-CoV-2 (4×10⁴ plaque forming units). After a 48 hour incubation period, both antiviral and cytotoxic plitidepsin effects were determined. Results showed that plitidepsin completely eliminated replication of SARS-CoV-2 with no observable cytotoxicity against the pneumocyte like cells.

Plitidepsin Effects on SARS-CoV-2 In Vivo

Plitidepsin demonstrated potent antiviral effects in vivo, using a previously described mouse model of adenovirus-mediated hACE2 infected with SARS-CoV-2. Plitidepsin also demonstrated potent antiviral effects in vivo using a previously described model of transgenic mice expressing hACE2 driven by the cytokeratin-18 gene promoter (K18-hACE2) infected with SARS-CoV-2.

Plitidepsin Effects on Host Inflammatory Reaction

Similar to SARS CoV, infection with SARS-CoV-2 also produces hypersecretion of several cytokines, with increasing plasma levels as the disease progresses, suggesting a possible relation between cytokine release and disease severity.

Innate immunity is the first line of defence against invading pathogens. In the case of SARS-CoV-2, the entry of the virus into host epithelial cells is mediated by the interaction between the viral envelope spike (S) protein and the cell surface receptor ACE2. Viral RNAs, as pathogen associated molecular patterns, are then detected by the host pattern recognition receptors, which include the family of toll like receptors. Toll like receptors then upregulate antiviral and proinflammatory mediators, such as interleukin (IL) 6, IL 8, and interferon (IFN)-γ, through activation of the transcription factor nuclear factor kappa B (NF-κB). The importance of NF-κB towards proinflammatory gene expression, particularly in the lungs, has been highlighted by studies exploring SARS CoV infection in nonclinical species as well as in patients. In mice infected with SARS CoV, the pharmacologic inhibition of NF-κB resulted in higher survival rates and reduced expression of tumour necrosis factor alpha (TNFα), CCL2, and CXCL2 in lungs.

Early in vitro studies showed that plitidepsin induces down-regulation of NFκB in tumour cells. Subsequently, both in vitro and ex vivo studies were performed to assess the effects of plitidepsin on immune cells.

In vitro studies were performed using THP-1 cells, a spontaneously immortalised monocyte-like cell line derived from the peripheral blood of a childhood case of acute monocytic leukaemia, that is widely used for investigating monocyte structure and function. Results showed that all the pathogen-associated molecular patterns-mimicking compounds induced the production of proinflammatory cytokines in THP-1 cells and the addition of plitidepsin significantly reduced the secretion of the proinflammatory cytokines.

An ex vivo study assessed the effect of plitidepsin on expression of the cytokines IL 6, IL 10, and TNFα in the lungs of mice. Results showed that cluster of differentiation (CD)45+ cells from placebo treated mice were capable of producing IL 6, IL 10, and TNFα upon LPS-B5 stimulation. However, CD45+ cells from plitidepsin treated mice failed to show a marked increase in IL 6, IL 10, and TNFα compared with nonstimulated controls. These results suggest that the in vivo exposure to plitidepsin prevented the increased production of proinflammatory cytokines mediated by LPS-B5 in the CD45+ cells isolated from broncho-alveolar lavages.

Compounds of the invention may be used in pharmaceutical compositions having biological/pharmacological activity for the treatment of the above mentioned infections and associated conditions. These pharmaceutical compositions comprise a compound of the invention together with a pharmaceutically acceptable carrier. The term “carrier” refers to a diluent, adjuvant, excipient or vehicle with which the active ingredient is administered. Suitable pharmaceutical carriers are described in “Remington's Pharmaceutical Sciences” by E. W. Martin, 1995. Examples of pharmaceutical compositions include any solid (tablets, pills, capsules, granules, etc.) or liquid (solutions, suspensions, emulsions, etc.) compositions for oral, topical or parenteral administration. Pharmaceutical compositions containing compounds of the invention may be delivered by liposome or nanosphere encapsulation, in sustained release formulations or by other standard delivery means.

An exemplary composition is in the form of powder for solution for infusion. For example, compositions as described in WO9942125. For example, a lyophilised preparation of a compound of the invention including water-soluble material and secondly a reconstitution solution of mixed solvents. A particular example is a lyophilised preparation of PLD and mannitol and a reconstitution solution of mixed solvents, for example PEG-35 castor oil, ethanol and water for injections. Each vial, for example may contain 2 mg of PLD. After reconstitution, each mL of reconstituted solution may contain: 0.5 mg of PLD, 158 mg of PEG-35 castor oil, and ethanol 0.15 mL/mL.

The specific dosage and treatment regimen for any particular patient may be varied and will depend upon a variety of factors, including the activity of the specific compound employed, the particular formulation being used, the mode of application, the age, body weight, general health, sex, diet, time of administration, rate of excretion, drug combination, reaction sensitivities, and the severity of the particular disease or condition being treated.

In embodiments of the invention, the compounds of the present invention may be administered according to a dosing regimen of a daily dose.

In embodiments of the invention, the compounds of the present invention may be administered according to a dosing regimen of a once daily dose.

In further embodiments, the compounds of the present invention may be administered according to a dosing regimen of a daily dose for 10 days, 9 days, 8 days, 7 days, 6 days, 5 days, 4 days, 3 days, 2 days or 1 day. Preferred regimen is 2-5 days, or 3-5 days, or 3, 4 or 5 days, most preferably 3 days or 5 days.

The dose may be a dose of 5 mg a day or less, 4.5 mg a day or less, 4 mg a day or less, 3.5 mg a day or less, 3 mg a day or less, 2.5 mg a day or less or 2 mg a day or less.

Particular doses include 0.5 mg/day, 1 mg/day, 1.5 mg/day, 2 mg/day, 2.5 mg/day, 3 mg/day, 3.5 mg/day, 4 mg/day, 4.5 mg/day, or 5 mg/day. Preferred doses are 1 mg/day, 1.5 mg/day, 2 mg/day and 2.5 mg/day.

In further embodiments, the compounds of the present invention may be administered according to a total dose of 1-50 mg, 1-40 mg, 1-30 mg, 1-20 mg, 1-15 mg, 3-15 mg, 3-12 mg, 4-12 mg, 4-10 mg, or 4.5-10 mg. Total doses may be 4 mg, 4.5 mg, 5 mg, 5.5 mg, 6 mg, 6.5 mg, 7 mg, 7.5 mg, 8 mg, 8.5 mg, 9 mg, 9.5 mg or 10 mg. Preferred total doses are 4.5 mg, 5 mg, 6 mg, 7.5 mg, 8 mg, 9 mg or 10 mg. The total dose may be split over 1, 2, 3, 4, 5, 6, 7, 8, 9 or 10 days, preferably 3 days or 5 days.

In a particular embodiment, the compounds of the present invention may be administered according to a dosing regimen of a once daily dose for 5 days, at a dose of 2.5 mg a day or less.

In a further embodiment, the compounds of the present invention may be administered according to a dosing regimen of a once daily dose for 5 days, at a dose of 2 mg a day or less.

In a further embodiment, the compounds of the present invention may be administered according to a dosing regimen of a once daily dose for 3 days, at a dose of 1.5 mg a day or less.

In a further embodiment, the compounds of the present invention may be administered according to a dosing regimen of a once daily dose for 3 days, at a dose of 2 mg a day or less.

In a further embodiment, the compounds of the present invention may be administered according to a dosing regimen of a once daily dose for 3 days, at a dose of 2.5 mg a day or less.

In a further embodiment, the compounds of the present invention may be administered according to a dosing regimen of a once daily dose for 3 days, at a dose of 1.5 mg a day.

In a further embodiment, the compounds of the present invention may be administered according to a dosing regimen of a once daily dose for 3 days, at a dose of 2.0 mg a day.

In a further embodiment, the compounds of the present invention may be administered according to a dosing regimen of a once daily dose for 3 days, at a dose of 2.5 mg a day.

In a further embodiment, the compounds of the present invention may be administered according to a dosing regimen of a once daily dose for 3 days, at a dose of 1.5 to 2.5 mg a day.

An alternative regimen is a single dose on day 1. The single dose regiment may be particularly suited to the treatment of: mild infection; reducing complications associated with CoV infection, including hospitalization, ICU and death; prophylaxis, reduction, avoidance or treatment of COVID persistent, long COVID, post-COVID syndrome; and/or reducing the infectivity of CoV patients. The single dose may be 1-10 mg, 4-10 mg, 4.5-10 mg; 4 mg, 4.5 mg, 5 mg, 5.5 mg, 6 mg, 6.5 mg, 7 mg, 7.5 mg, 8 mg, 8.5 mg, 9 mg, 9.5 mg or 10 mg; preferably 4.5 mg, 5 mg, 6 mg, 7.5 mg, 8 mg, 9 mg or 10 mg; more preferably 5-9 mg, 6.5-8.5 mg, 7-8 mg or 7.5 mg.

In a further embodiment, the compounds of the present invention may be administered according to the present invention, wherein the compounds of the present invention are administered with a corticosteroid. Preferably the corticosteroid is dexamethasone.

The corticosteroid may be administered daily with the compounds of the present invention. Administration may be sequential, concurrent or consecutive. The corticosteroid may be further administered on the days following administration of compounds according to the present invention. By way of example, with a 3 day dosing regimen, the corticosteroid may be administered on days 1-3 and then further administered daily for 3, 4, 5, 6, 7, 8, 9 or 10 or more further days.

In a particular embodiment, the corticosteroid may be administered is administered on days 1-3 as an intravenous administration and then on days 6-10 as an oral administration. In a further embodiment, the dosage of corticosteroid may be higher during the co-administration phase with the compounds of the present invention, and is lowered during the subsequent days.

Particular dosing schedules include:
Plitidepsin 1.5 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).
Plitidepsin 2.0 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).
Plitidepsin 2.5 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).

In an embodiment, to avoid administration-related infusion reactions patients may receive the following medications 20 to 30 minutes prior to starting the infusion with a compound according to the present invention:
Ondansetron 8 mg IV (or equivalent)
Diphenhydramine hydrochloride 25 mg IV (or equivalent)
Ranitidine 50 mg IV (or equivalent)
Dexamethasone 6.6 mg IV (which is included in the schedule above)

Additionally, on Days 4 and 5 patients treated with compounds according to the present invention may receive ondansetron 4 mg twice a day PO.

Doses of dexamethasone, ondansetron and ranitidine are herein defined on the basis of the base form. The dose of diphenhydramine hydrochloride is given on the basis of the hydrochloride salt. Doses of compounds of the invention are given on the basis of the base form.

The daily doses may be administered as an infusion. The infusion may be a 1 hour infusion, a 1.5 hour infusion, a 2 hour infusion, a 3 hour infusion or longer. Preferably, the infusion is 1.5 hours.

In certain embodiments, the dose may be administered according to a regimen which uses a loading dose and a maintenance dose. Loading/maintenance doses according to the present invention includes:
a loading dose of 2.5 mg for day 1, and followed by a maintenance dose of 2 mg/day for subsequent days;
a loading dose of 2.5 mg for day 1, and followed by a maintenance dose of 1.5 mg/day for subsequent days;
a loading dose of 2.5 mg for day 1, and followed by a maintenance dose of 1 mg/day for subsequent days;
a loading dose of 2.5 mg for day 1, and followed by a maintenance dose of 0.5 mg/day for subsequent days;
a loading dose of 2 mg for day 1, and followed by a maintenance dose of 1.5 mg/day for subsequent days;
a loading dose of 2 mg for day 1, and followed by a maintenance dose of 1 mg/day for subsequent days;
a loading dose of 2 mg for day 1, and followed by a maintenance dose of 0.5 mg/day for subsequent days;
a loading dose of 1.5 mg for day 1, and followed by a maintenance dose of 1 mg/day for subsequent days;
a loading dose of 1.5 mg for day 1, and followed by a maintenance dose of 0.5 mg/day for subsequent days; and
a loading dose of 1 mg for day 1, and followed by a maintenance dose of 0.5 mg/day for subsequent days.

According to a further embodiment, the daily dose may be reduced in the final day or days of the regimen.

According to a further embodiment, if the daily dose is 2 mg, the dose may be reduced to 1 mg on days 4 and 5.

Particular regimens include:
1 mg of plitidepsin administered as a 1.5-hour infusion, once a day for 5 consecutive days. (total dose 5 mg);
2 mg of plitidepsin administered as a 1.5-hour infusion, once a day for 5 consecutive days. At the discretion of the researcher, the dose may be reduced to 1 mg/day on days 4 and 5 (total dose 8-10 mg);
1.5 mg of plitidepsin administered as a 1.5-hour infusion, once a day for 3 consecutive days. (total dose 4.5 mg);
2 mg of plitidepsin administered in 1.5 hour infusion, once a day for 3 consecutive days. (total dose 6 mg); and
2.5 mg of plitidepsin administered as a 1.5-hour infusion, once a day for 3 consecutive days. (total dose 7.5 mg).

A single dose regimen includes:
Plitidepsin administered as a 1.5-hour infusion, once on day 1 at a dose of 1-10 mg, 4-10 mg, 4.5-10 mg, 4 mg, 4.5 mg, 5 mg, 5.5 mg, 6 mg, 6.5 mg, 7 mg, 7.5 mg, 8 mg, 8.5 mg, 9 mg, 9.5 mg or 10 mg, preferably 4.5 mg, 5 mg, 6 mg, 7.5 mg, 8 mg, 9 mg or 10 mg, more preferably 5-9 mg, 6.5-8.5 mg, 7-8 mg or most preferably 7.5 mg.
The single dose regimen may further include the following prophylactic medications 20-30 minutes prior to plitidepsin infusion:
   Diphenhydramine hydrochloride 25 mg i.v.,
   Ranitidine 50 mg i.v.
   Dexamethasone 6.6 mg intravenously.
   Ondansetron 8 mg i.v. in slow infusion of 15 minutes
Ondansetron 4 mg orally may be given every 12 hours for 3 days after plitidepsin administration to relieve drug-induced nausea and vomiting. If plitidepsin is administered in the morning the patient may receive the first dose of ondansetron in the afternoon.

According to further embodiments, patients may be selected for treatment with plitidepsin based on clinical parameters and/or patient characteristics. Suitable parameters may be measurements disclosed in the present application.

The regimens and doses outlined above apply to both methods of treatment according to the present invention, use, and use of a compound as defined herein in the manufacture of medicaments as defined herein.

In embodiments, the present invention is directed to a compound for use according to the present invention, wherein the compound is administered in combination with one or more of the following prophylactic medications: diphenhydramine hydrochloride, ranitidine, dexamethasone, ondansetron. In particular, one or more of diphenhydramine hydrochloride 25 mg iv or equivalent; Ranitidine 50 mg iv or equivalent; Dexamethasone 8 mg intravenous; Ondansetron 8 mg i.v. in slow infusion of 15 minutes or equivalent. Patients may receive said prophylactic medications 20-30 minutes before the infusion of plitidepsin. Dexamethasone 8 mg intravenous may be dexamethasone phosphate leading to 6.6 mg dexamethasone base.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof.

However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

### EXAMPLES

Compounds of the present invention can be obtained according to the processes set out in the literature, for example: Vera et al. Med. Res. Rev. 2002, 22(2), 102-145, WO 2011/020913 (see in particular Examples 1-5), WO 02/02596, WO 01/76616, and WO 2004/084812, the contents of which are incorporated herein by reference.

Particular compounds used in experiments of the present invention are:

| Compound | Structure |
|---|---|
| PLD | 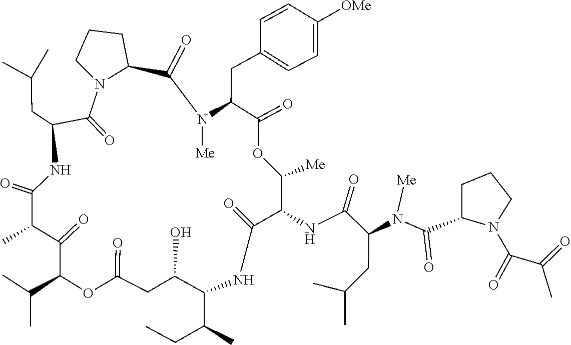 [SMILES: CC[C@H](C)[C@H]1NC(=O)[C@@H](NC(=O)[C@H](CC(C)C)N(C)C(=O)[C@@H]2CCCN2C(=O)C(C)=O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O] |
| DidemninB (compound 240) | 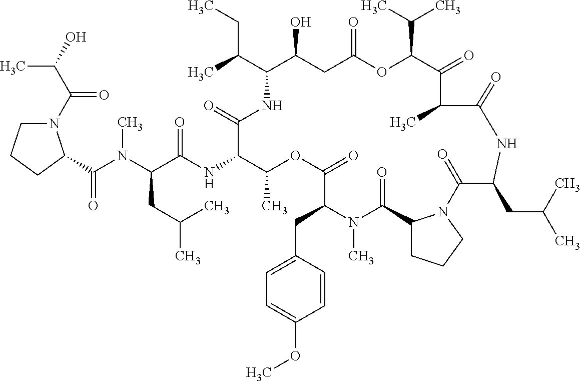 |
| Compound 3 | 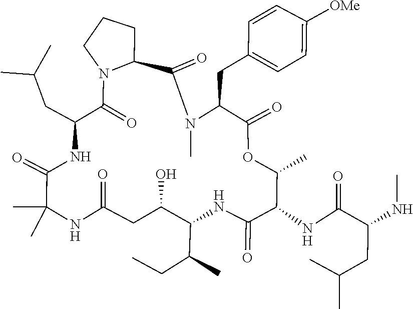 [SMILES: CC[C@H](C)[C@H]1NC(=O)[C@@H](NC(=O)[C@@H](CC(C)C)NC)[C@@H](C)OC(=O)[C@H](Cc2ccc(OC)cc2)N(C)C(=O)[C@@H]2CCCN2C(=O)[C@H](CC(C)C)NC(=O)C(C)(C)NC(=O)C[C@@H]1O] |
| Compound 8 | 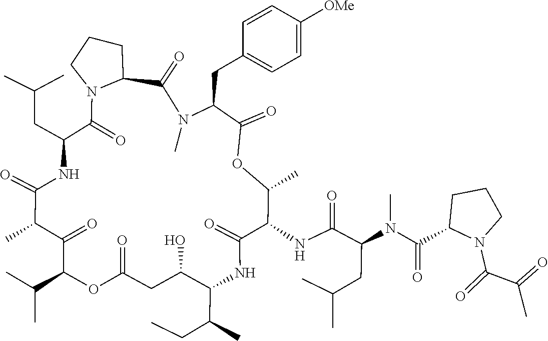 [SMILES: CC[C@H](C)[C@H]1NC(=O)[C@@H](NC(=O)[C@H](CC(C)C)N(C)C(=O)[C@@H]2CCCN2C(=O)C(C)=O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O] |
| Compound 9 | 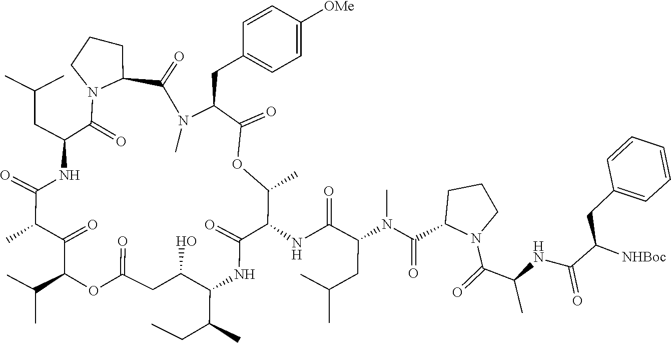 [SMILES: CC[C@H](C)[C@H]1NC(=O)[C@@H](NC(=O)[C@@H](CC(C)C)N(C)C(=O)[C@@H]2CCCN2C(=O)[C@H](C)NC(=O)[C@@H](Cc2ccccc2)NC(=O)OC(C)(C)C)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O] |
| Compound 10 | 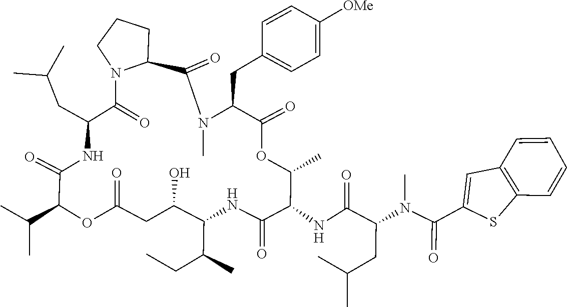 [SMILES: CC[C@H](C)[C@H]1NC(=O)[C@@H](NC(=O)[C@@H](CC(C)C)N(C)C(=O)c2cc3ccccc3s2)[C@@H](C)OC(=O)[C@H](Cc2ccc(OC)cc2)N(C)C(=O)[C@@H]2CCCN2C(=O)[C@H](CC(C)C)NC(=O)[C@H](C(C)C)OC(=O)C[C@@H]1O] |
| Compound 11 | 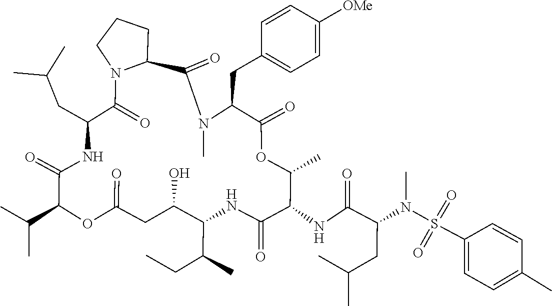 [SMILES: CC[C@H](C)[C@H]1NC(=O)[C@@H](NC(=O)[C@@H](CC(C)C)N(C)S(=O)(=O)c2ccc(C)cc2)[C@@H](C)OC(=O)[C@H](Cc2ccc(OC)cc2)N(C)C(=O)[C@@H]2CCCN2C(=O)[C@H](CC(C)C)NC(=O)[C@H](C(C)C)OC(=O)C[C@@H]1O] |

Following the procedures described in WO 02/02596 and in the specification, and further disclosed in the previous examples, the following compounds are obtainable:

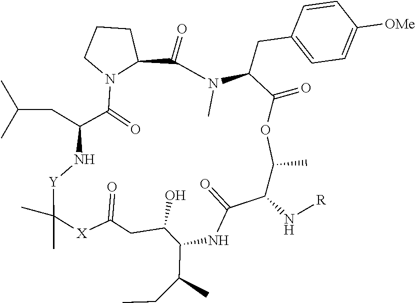

| Compound | X | Y | R |
|---|---|---|---|
| 12 13 14 15 | O NH O NH | CO CO —COCH(CH₃)CO— —COCH(CH₃)CO— | 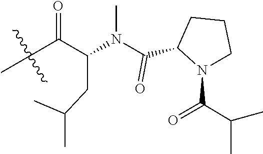 |
| 16 17 18 19 | O NH O NH | CO CO —COCH(CH₃)CO— —COCH(CH₃)CO— | 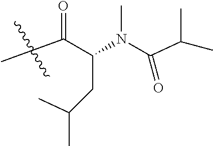 [SMILES: *C(=O)[C@@H](CC(C)C)N(C)C(=O)C(C)C] |
| 20 21 22 23 | O NH O NH | CO CO —COCH(CH₃)CO— —COCH(CH₃)CO— | 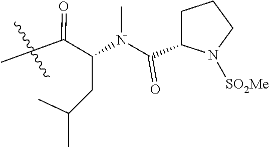 [SMILES: *C(=O)[C@@H](CC(C)C)N(C)C(=O)[C@@H]1CCCN1S(C)(=O)=O] |
| 24 25 26 27 | O NH O NH | CO CO —COCH(CH₃)CO— —COCH(CH₃)CO— | 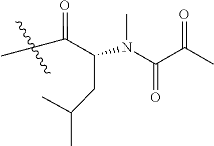 [SMILES: *C(=O)[C@@H](CC(C)C)N(C)C(=O)C(C)=O] |
| 28 29 30 31 | O NH O NH | CO CO —COCH(CH₃)CO— —COCH(CH₃)CO— | 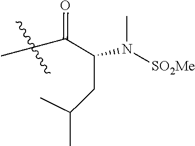 [SMILES: *C(=O)[C@@H](CC(C)C)N(C)S(C)(=O)=O] |
| 32 33 34 35 | O NH O NH | CO CO —COCH(CH₃)CO— —COCH(CH₃)CO— | 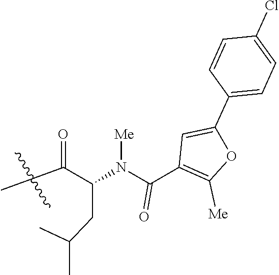 [SMILES: *C(=O)[C@@H](CC(C)C)N(C)C(=O)c1cc(-c2ccc(Cl)cc2)oc1C] |
| 36 37 38 39 | O NH O NH | CO CO —COCH(CH₃)CO— —COCH(CH₃)CO— | 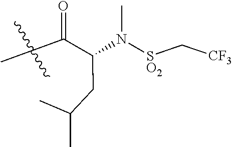 [SMILES: *C(=O)[C@@H](CC(C)C)N(C)S(=O)(=O)CC(F)(F)F] |
| 40 41 42 43 | O NH O NH | CO CO —COCH(CH₃)CO— —COCH(CH₃)CO— | 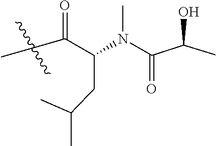 [SMILES: *C(=O)[C@@H](CC(C)C)N(C)C(=O)[C@H](C)O] |
| 44 45 46 47 | O NH O NH | CO CO —COCH(CH₃)CO— —COCH(CH₃)CO— | 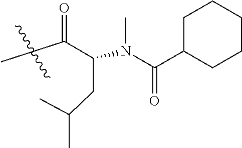 [SMILES: *C(=O)[C@@H](CC(C)C)N(C)C(=O)C1CCCCC1] |
| 48 49 50 51 | O NH O NH | CO CO —COCH(CH₃)CO— —COCH(CH₃)CO— | 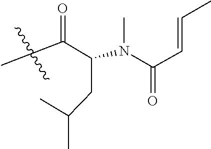 [SMILES: *C(=O)[C@@H](CC(C)C)N(C)C(=O)/C=C/C] |
| 52 53 54 55 | O NH O NH | CO CO —COCH(CH₃)CO— —COCH(CH₃)CO— | 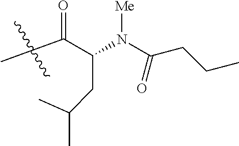 [SMILES: *C(=O)[C@@H](CC(C)C)N(C)C(=O)CCC] |
| 56 57 58 59 | O NH O NH | CO CO —COCH(CH₃)CO— —COCH(CH₃)CO— | 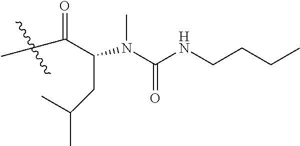 [SMILES: *C(=O)[C@@H](CC(C)C)N(C)C(=O)NCCCC] |
| 60 61 62 63 | O NH O NH | CO CO —COCH(CH₃)CO— —COCH(CH₃)CO— | 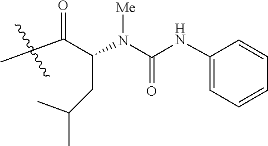 [SMILES: *C(=O)[C@@H](CC(C)C)N(C)C(=O)Nc1ccccc1] |
| 64 65 66 67 | O NH O NH | CO CO —COCH(CH₃)CO— —COCH(CH₃)CO— | 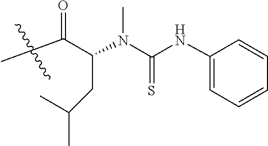 [SMILES: *C(=O)[C@@H](CC(C)C)N(C)C(=S)Nc1ccccc1] |
| 68 69 70 71 | O NH O NH | CO CO —COCH(CH₃)CO— —COCH(CH₃)CO— | 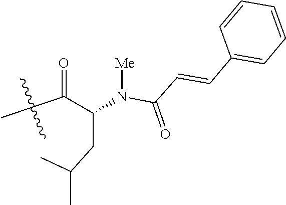 [SMILES: *C(=O)[C@@H](CC(C)C)N(C)C(=O)/C=C/c1ccccc1] |
| 72 73 74 75 | O NH O NH | CO CO —COCH(CH₃)CO— —COCH(CH₃)CO— | 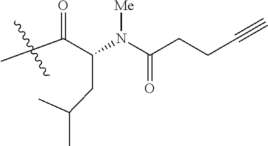 [SMILES: *C(=O)[C@@H](CC(C)C)N(C)C(=O)CCC#C] |
| 76 77 78 79 | O NH O NH | CO CO —COCH(CH₃)CO— —COCH(CH₃)CO— | 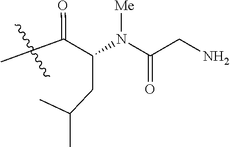 [SMILES: *C(=O)[C@@H](CC(C)C)N(C)C(=O)CN] |
| 80 81 82 83 | O NH O NH | CO CO —COCH(CH₃)CO— —COCH(CH₃)CO— | 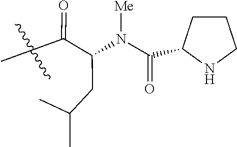 [SMILES: *C(=O)[C@@H](CC(C)C)N(C)C(=O)[C@@H]1CCCN1] |
| 84 85 86 87 | O NH O NH | CO CO —COCH(CH₃)CO— —COCH(CH₃)CO— | 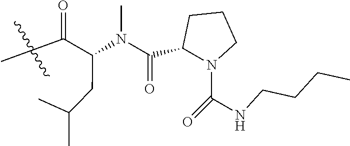 [SMILES: *C(=O)[C@@H](CC(C)C)N(C)C(=O)[C@@H]1CCCN1C(=O)NCCCC] |
| 88 89 90 91 | O NH O NH | CO CO —COCH(CH₃)CO— —COCH(CH₃)CO— | 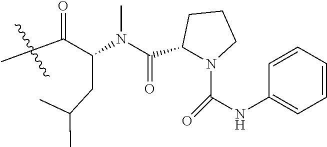 [SMILES: *C(=O)[C@@H](CC(C)C)N(C)C(=O)[C@@H]1CCCN1C(=O)Nc1ccccc1] |
| 92 93 94 95 | O NH O NH | CO CO —COCH(CH₃)CO— —COCH(CH₃)CO— | 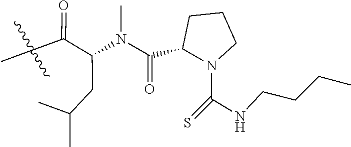 [SMILES: *C(=O)[C@@H](CC(C)C)N(C)C(=O)[C@@H]1CCCN1C(=S)NCCCC] |
| 96 97 98 99 | O NH O NH | CO CO —COCH(CH₃)CO— —COCH(CH₃)CO— | 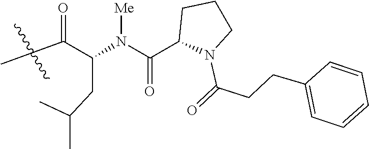 [SMILES: *C(=O)[C@@H](CC(C)C)N(C)C(=O)[C@@H]1CCCN1C(=O)CCc1ccccc1] |
| 100 101 102 103 | O NH O NH | CO CO —COCH(CH₃)CO— —COCH(CH₃)CO— | 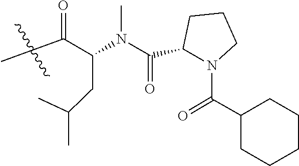 [SMILES: *C(=O)[C@@H](CC(C)C)N(C)C(=O)[C@@H]1CCCN1C(=O)C1CCCCC1] |
| 104 105 106 107 | O NH O NH | CO CO —COCH(CH₃)CO— —COCH(CH₃)CO— | 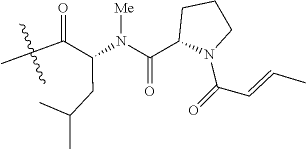 [SMILES: *C(=O)[C@@H](CC(C)C)N(C)C(=O)[C@@H]1CCCN1C(=O)/C=C/C] |
| 108 109 110 111 | O NH O NH | CO CO —COCH(CH₃)CO— —COCH(CH₃)CO— | 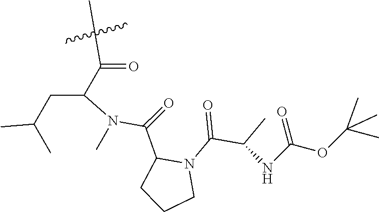 [SMILES: *C(=O)C(CC(C)C)N(C)C(=O)C1CCCN1C(=O)[C@H](C)NC(=O)OC(C)(C)C] |
| 112 113 114 115 | O NH O NH | CO CO —COCH(CH₃)CO— —COCH(CH₃)CO— | 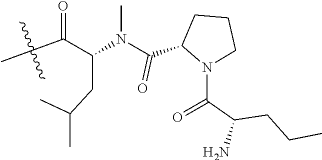 [SMILES: *C(=O)[C@@H](CC(C)C)N(C)C(=O)[C@@H]1CCCN1C(=O)[C@@H](N)CCC] |
| 116 117 118 119 | O NH O NH | CO CO —COCH(CH₃)CO— —COCH(CH₃)CO— | 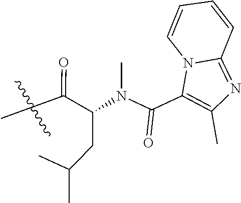 [SMILES: *C(=O)[C@@H](CC(C)C)N(C)C(=O)c1c(C)nc2ccccn12] |
| 120 121 122 123 | O NH O NH | CO CO —COCH(CH₃)CO— —COCH(CH₃)CO— | 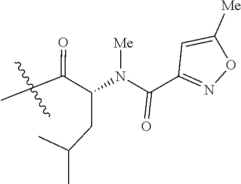 [SMILES: *C(=O)[C@@H](CC(C)C)N(C)C(=O)c1cc(C)on1] |
| 124 125 126 127 | O NH O NH | CO CO —COCH(CH₃)CO— —COCH(CH₃)CO— | 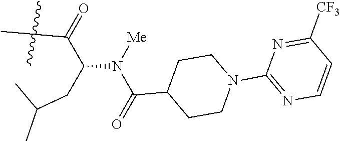 [SMILES: *C(=O)[C@@H](CC(C)C)N(C)C(=O)C1CCN(c2nccc(C(F)(F)F)n2)CC1] |
| 128 129 130 131 | O NH O NH | CO CO —COCH(CH₃)CO— —COCH(CH₃)CO— | 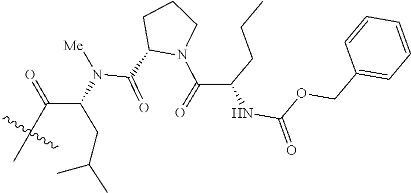 [SMILES: *C(=O)[C@@H](CC(C)C)N(C)C(=O)[C@@H]1CCCN1C(=O)[C@H](CCC)NC(=O)OCc1ccccc1] |

Following the procedures described in WO 02/02596 and in the specification, and further disclosed n the previous examples. the following compounds are obtainable:

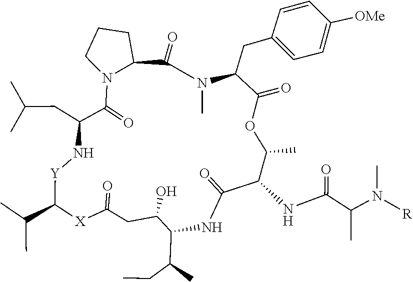

| Com- | | | |
|---|---|---|---|
| pound | X | Y | R |
| 132 133 134 135 | O NH O NH | CO CO —COCH(CH₃)CO— —COCH(CH₃)CO— | 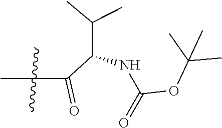 [SMILES: *C(=O)[C@@H](NC(=O)OC(C)(C)C)C(C)C] |
| 136 137 138 139 | O NH O NH | CO CO —COCH(CH₃)CO— —COCH(CH₃)CO— | 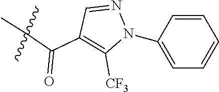 [SMILES: *C(=O)c1cnn(-c2ccccc2)c1C(F)(F)F] |
| 140 141 142 143 | O NH O NH | CO CO —COCH(CH₃)CO— —COCH(CH₃)CO— | 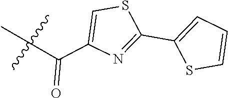 [SMILES: *C(=O)c1csc(-c2cccs2)n1] |
| 144 145 146 147 | O NH O NH | CO CO —COCH(CH₃)CO— —COCH(CH₃)CO— | 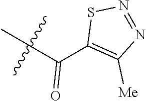 [SMILES: *C(=O)c1snnc1C] |
| 148 149 150 151 | O NH O NH | CO CO —COCH(CH₃)CO— —COCH(CH₃)CO— | 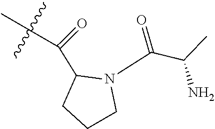 [SMILES: *C(=O)C1CCCN1C(=O)[C@H](C)N] |
| 152 153 154 155 | O NH O NH | CO CO —COCH(CH₃)CO— —COCH(CH₃)CO— | 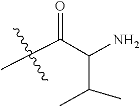 [SMILES: *C(=O)C(N)C(C)C] |
| 156 157 158 159 | O NH O NH | CO CO —COCH(CH₃)CO— —COCH(CH₃)CO— | 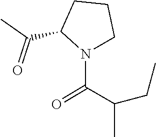 |
| 160 161 162 163 | O NH O NH | CO CO —COCH(CH₃)CO— —COCH(CH₃)CO— | 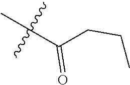 [SMILES: *C(=O)CCC] |
| 164 165 166 167 | O NH O NH | CO CO —COCH(CH₃)CO— —COCH(CH₃)CO— | 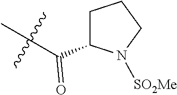 [SMILES: *C(=O)[C@@H]1CCCN1S(C)(=O)=O] |
| 168 169 170 171 | O NH O NH | CO CO —COCH(CH₃)CO— —COCH(CH₃)CO— | 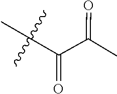 [SMILES: *C(=O)C(C)=O] |
| 172 173 174 175 | O NH O NH | CO CO —COCH(CH₃)CO— —COCH(CH₃)CO— | 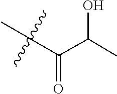 [SMILES: *C(=O)C(C)O] |
| 176 | O | CO | —SO₂Me |
| 177 | NH | CO | |
| 178 | O | —COCH(CH₃)CO— | |
| 179 | NH | —COCH(CH₃)CO— | |
| 180 181 182 183 | O NH O NH | CO CO —COCH(CH₃)CO— —COCH(CH₃)CO— | 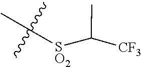 [SMILES: *S(=O)(=O)C(C)C(F)(F)F] |
| 184 185 186 187 | O NH O NH | CO CO —COCH(CH₃)CO— —COCH(CH₃)CO— | 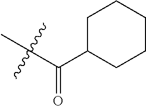 [SMILES: *C(=O)C1CCCCC1] |
| 188 189 190 191 | O NH O NH | CO CO —COCH(CH₃)CO— —COCH(CH₃)CO— | 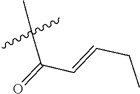 [SMILES: *C(=O)/C=C/CC] |
| 192 193 194 195 | O NH O NH | CO CO —COCH(CH₃)CO— —COCH(CH₃)CO— | 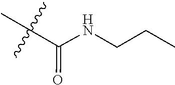 [SMILES: *C(=O)NCCC] |
| 196 197 198 199 | O NH O NH | CO CO —COCH(CH₃)CO— —COCH(CH₃)CO— | 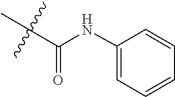 [SMILES: *C(=O)Nc1ccccc1] |
| 200 201 202 203 | O NH O NH | CO CO —COCH(CH₃)CO— —COCH(CH₃)CO— | 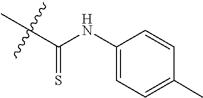 [SMILES: *C(=S)Nc1ccc(C)cc1] |
| 204 205 206 207 | O NH O NH | CO CO —COCH(CH₃)CO— —COCH(CH₃)CO— | 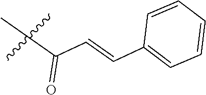 [SMILES: *C(=O)/C=C/c1ccccc1] |
| 208 209 210 211 | O NH O NH | CO CO —COCH(CH₃)CO— —COCH(CH₃)CO— | 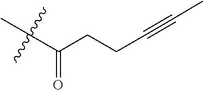 [SMILES: *C(=O)CCC#CC] |
| 212 213 214 215 | O NH O NH | CO CO —COCH(CH₃)CO— —COCH(CH₃)CO— | 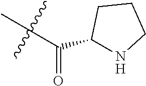 [SMILES: *C(=O)[C@@H]1CCCN1] |
| 216 217 218 219 | O NH O NH | CO CO —COCH(CH₃)CO— —COCH(CH₃)CO— | 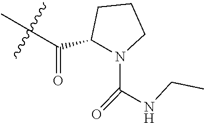 [SMILES: *C(=O)[C@@H]1CCCN1C(=O)NCC] |
| 220 221 222 223 | O NH O NH | CO CO —COCH(CH₃)CO— —COCH(CH₃)CO— | 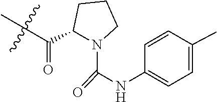 [SMILES: *C(=O)[C@@H]1CCCN1C(=O)Nc1ccc(C)cc1] |
| 224 225 226 227 | O NH O NH | CO CO —COCH(CH₃)CO— —COCH(CH₃)CO— | 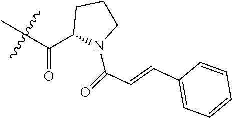 [SMILES: *C(=O)[C@@H]1CCCN1C(=O)/C=C/c1ccccc1] |
| 228 229 230 231 | O NH O NH | CO CO —COCH(CH₃)CO— —COCH(CH₃)CO— | 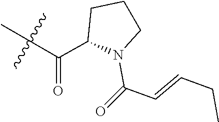 [SMILES: *C(=O)[C@@H]1CCCN1C(=O)/C=C/CC] |
| 232 233 234 235 | O NH O NH | CO CO —COCH(CH₃)CO— —COCH(CH₃)CO— | 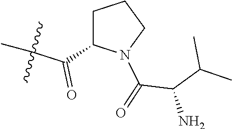 [SMILES: *C(=O)[C@@H]1CCCN1C(=O)[C@@H](N)C(C)C] |
| 236 237 238 239 | O NH O NH | CO CO —COCH(CH₃)CO— —COCH(CH₃)CO— | 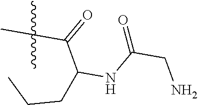 [SMILES: *C(=O)C(CCC)NC(=O)CN] |

A further compound is Compound 240, known as DidemninB and shown by the structure below:

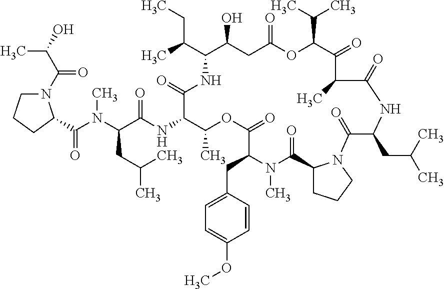 [SMILES: CC[C@H](C)[C@H]1NC(=O)[C@@H](NC(=O)[C@@H](CC(C)C)N(C)C(=O)[C@@H]2CCCN2C(=O)[C@H](C)O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O]

### Example 1

Recombinant virus assay was performed in both, MT-2 cells and PBMCs previously activated with PHA+IL-2. Cells were infected with supernatants obtained from 293t cells transfected with full-length infectious HIV-1 plasmids pNL4.3-Luc (X4 tropic virus), pNL4.3-Renilla (X4 tropic virus able to develop more than one round of replication), pNL4.3-Δenv-Luc plus pVSV-env (HIV pseudotyped with the G protein of VSV) or pJR-Renilla (R5 tropic virus able to develop more than one round of replication). Resistant viruses were obtained cloning in NL4.3-Renilla the pol gene of viruses from different infected donors. Virus 9D carry the following mutations: 41L, 67N, 70R, 98G, 118I, 184V, 215F, 219Q, 74I and virus 4D: K65R, K70R, V75I, F77L, F116Y, Q151M, M1841, L10I. The assay was then performed in 96 well microplates seeded with 100 μl containing 250.000 (PBMCs) or 100.000 (MT-2) cells/well. The compounds to be tested were added to the culture in concentrations ranging from 50 to 0.0016 pg/ml (100 μl/well). Finally, cell culture was infected with supernatants obtained form transfection of the different plasmids described above. After 48 hours, cell culture supernatant was removed and cells were lysed with Luciferase assay system or Renilla assay system (both from Promega) following the specifications of the manufacturer, and the luciferase-renilla activity was measured in a luminometre (Berthold Detection systems). All the experiments were controlled with cells treated with the vehicle (DMSO) and non-treated cells.

HIV-1 replication inhibition was evaluated by measuring the reduction of luciferase-renilla activity or RLUs (Relative light units) in a luminometre, being the 100% the infection of non-treated cells.

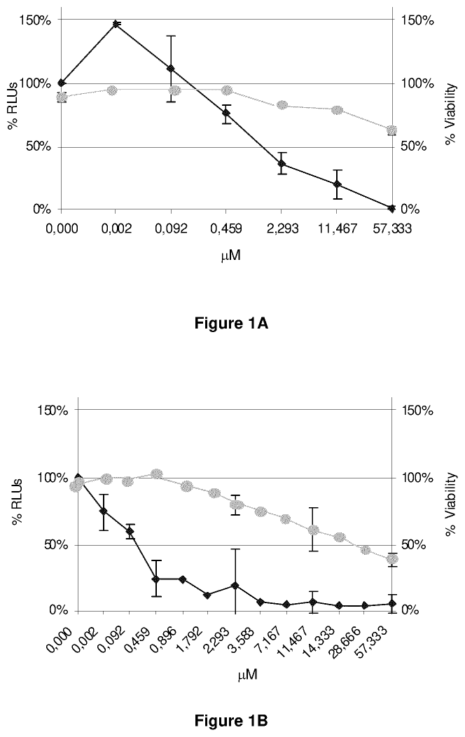

Compound 3 (FIGS. 1A and 1B) showed antiviral activity in both MT-2 cells and PBMCs (IC₅₀ 1.39 uM and 0.16 μM, respectively). This compound was more toxic in PBMCs, as shown in FIG. 1. Toxic concentrations were not reached at 57.3 μM in MT-2 cells, while in PBMCS CC₅₀ value was about 27 μM.

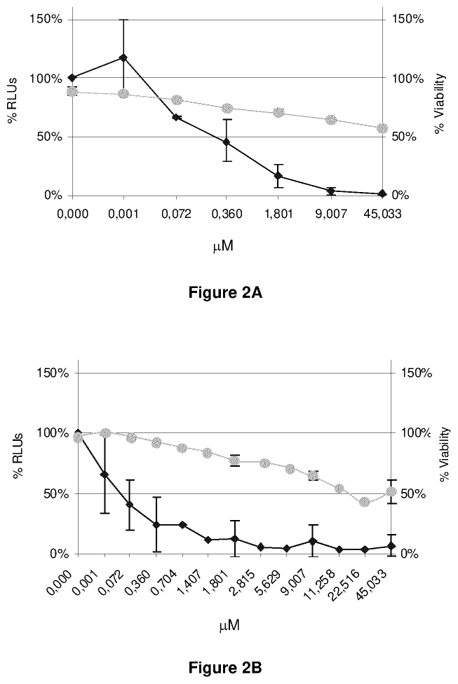

Compound 8 (FIGS. 2A and 2B) showed also antiviral activity in both MT-2 cells and PBMCs. Although at concentrations of 50 μM it was non-specific, at 10 μM it was specific, with an IC₅₀ value 100 fold lower.

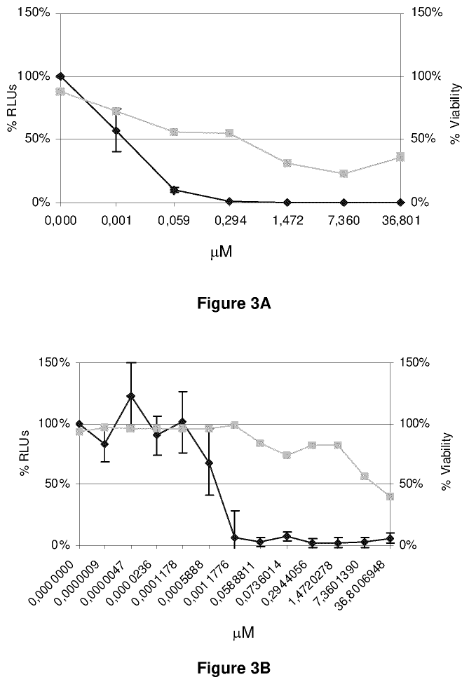

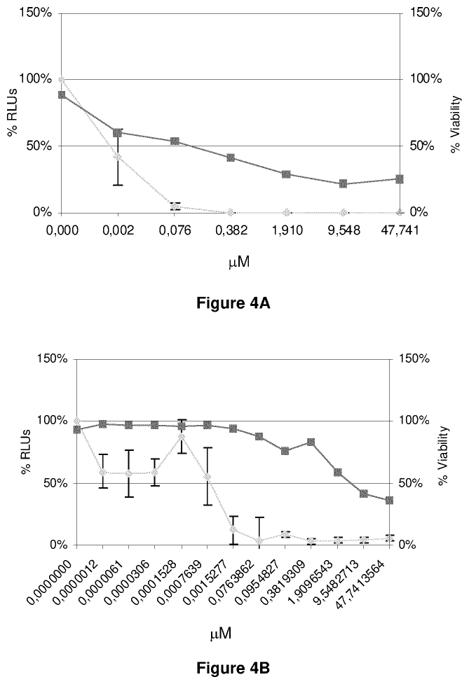

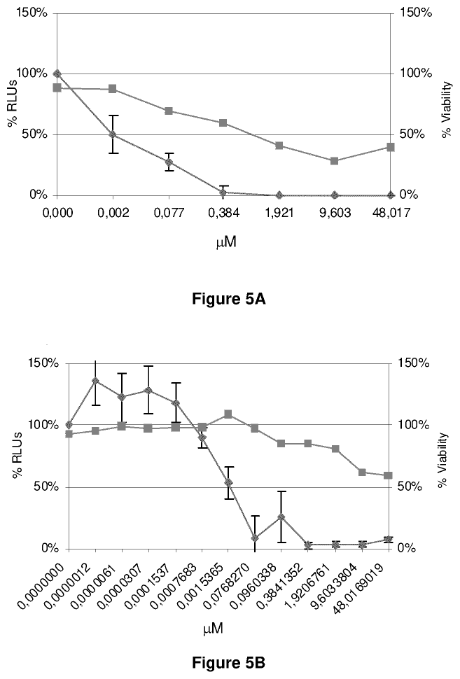

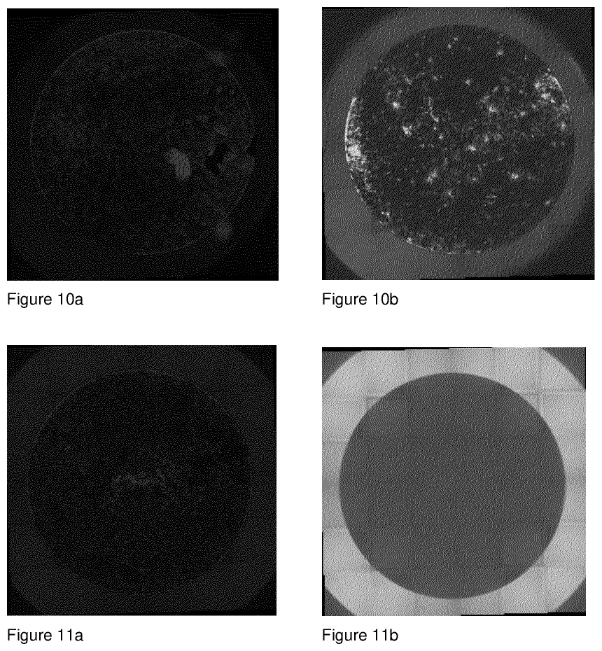

Compounds 9 (FIGS. 3A and 3B), 10 (FIGS. 4A and 4B), and 11 (FIGS. 5A and 5B) were the most potent compounds of all tested compounds. Compounds 9, 10, and 11 showed IC50s values in the nanomolar range in PBMCs (0.63, 0.86, and 69.4 nM, respectively), and they are among the most potent of the antiviral compounds in vitro existing in the literature.

### Example 2

Antiviral activity of compounds of the invention were tested in Huh-7 cells (human hepatoma cell line) infected with HCoV-229E. HCoV-229E has a multiplication and propagation mechanism very similar to SARS-COV-2. Indeed, the N protein of HCoV-229E has a protein homology greater than 90% with the homologous N protein in SARS-CoV-2. It is believed that all coronaviruses need their N (nucleocapsid) protein to bind to EF1A in order to replicate effectively and synthesise viral proteins. Reducing or abolishing the binding of N to EF1A reduces the viability for the spread of the virus.

Compounds of the invention as set out in Table 1 below were reconstituted in DMSO and stored at −20° C.

Huh-7 cells (human hepatoma cell line) grown to confluence in a M96 well plate, were infected with HCoV-229E-GFP virus at an moi (multiplicity of infection) of 0.01 pfu. Virus stock was HCoV-229E-GFP (from 31 Jan. 2013) at 3×10⁷ pfu/ml.

At 8 hpi (hours post infection), media is replaced by media with the appropriated compound dilutions (DMSO final concentration 2%), following the scheme:

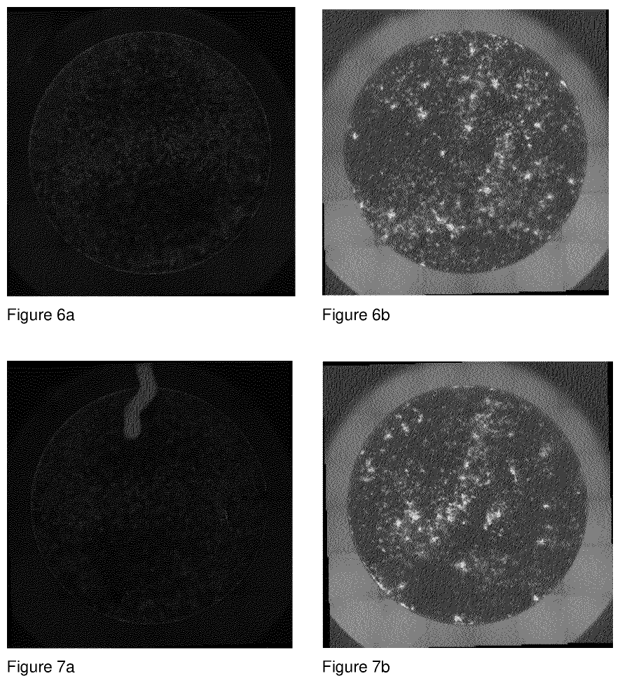

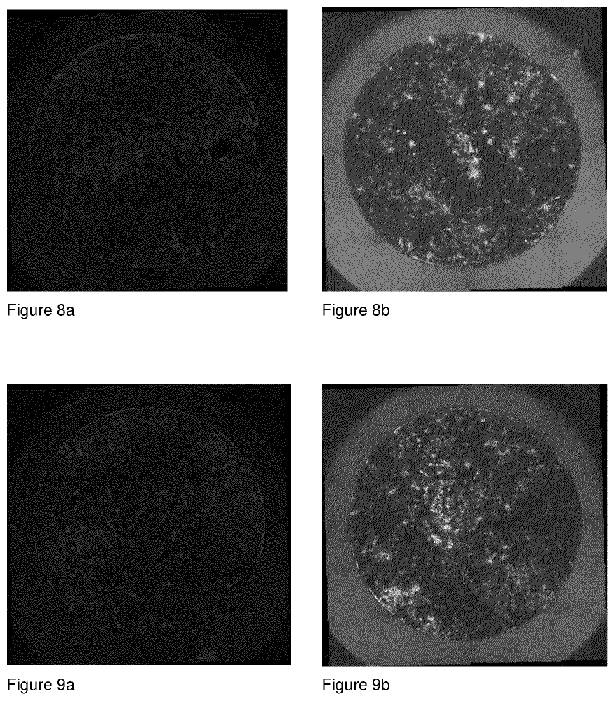

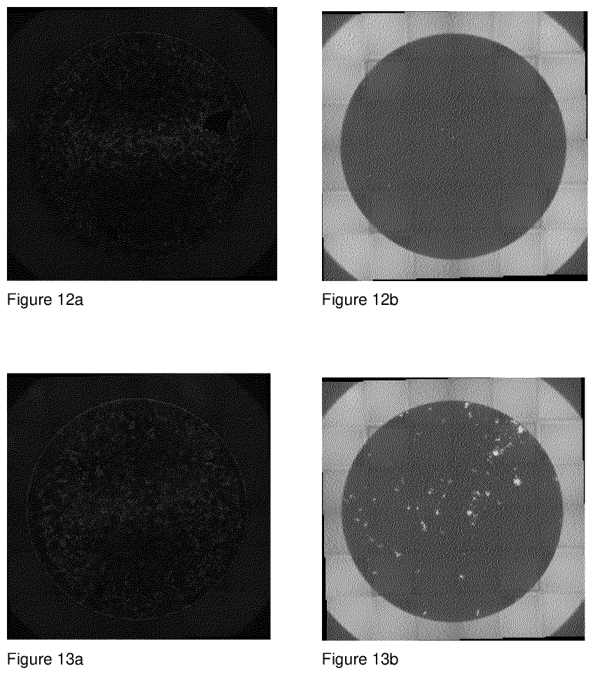

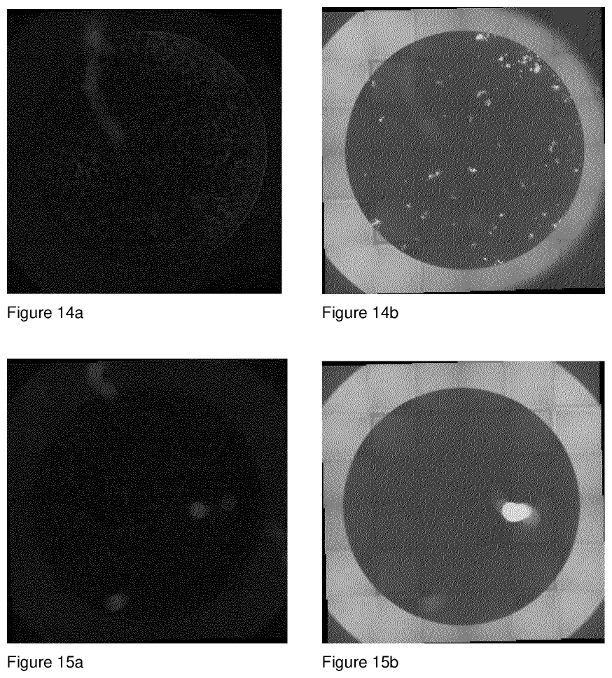

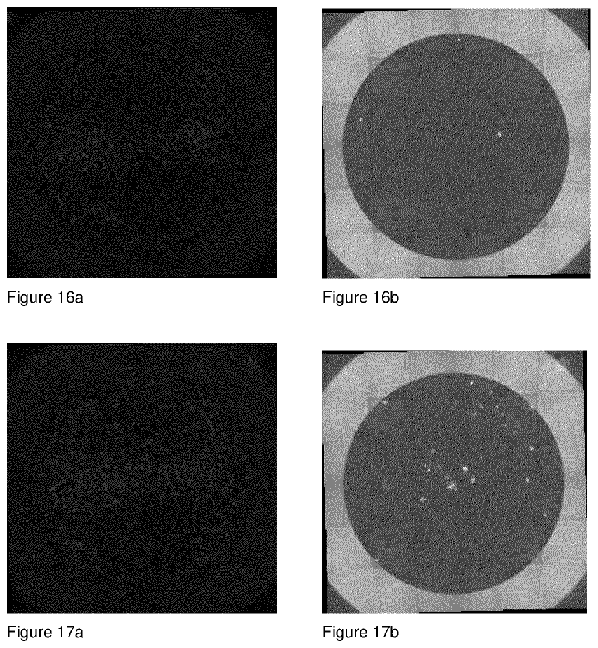

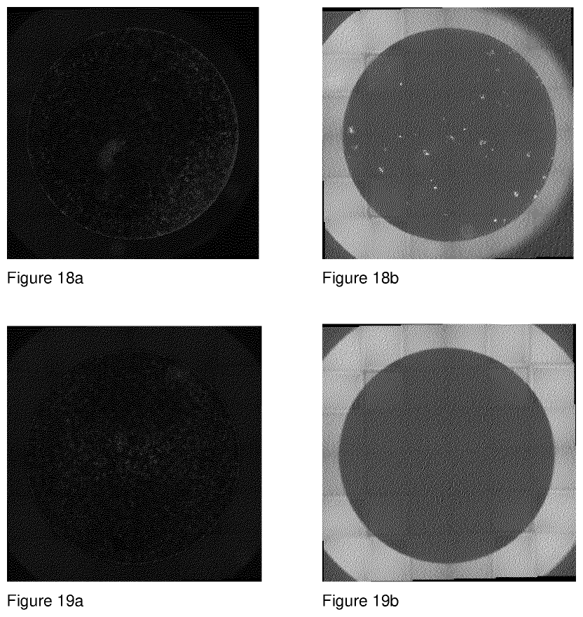

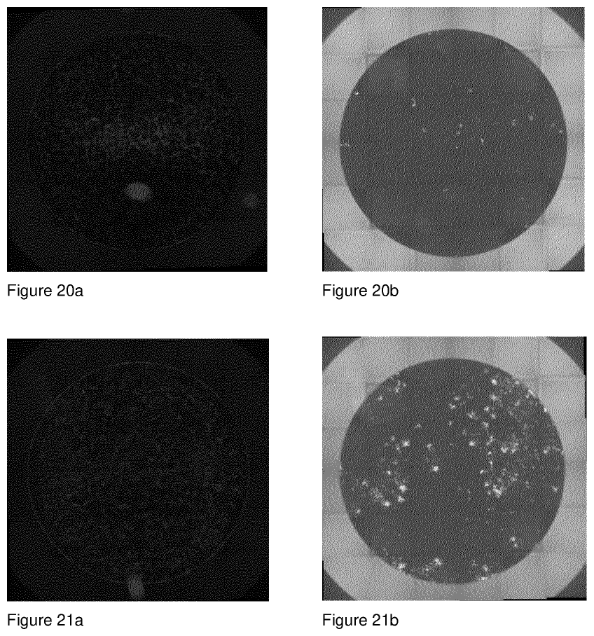

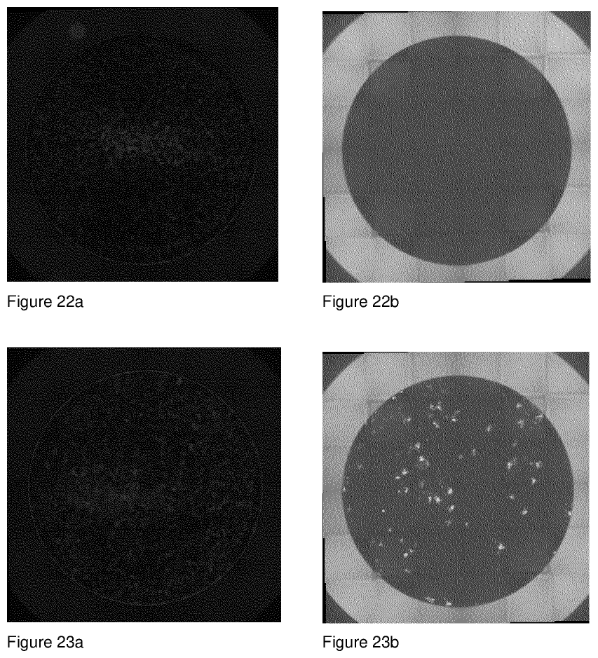

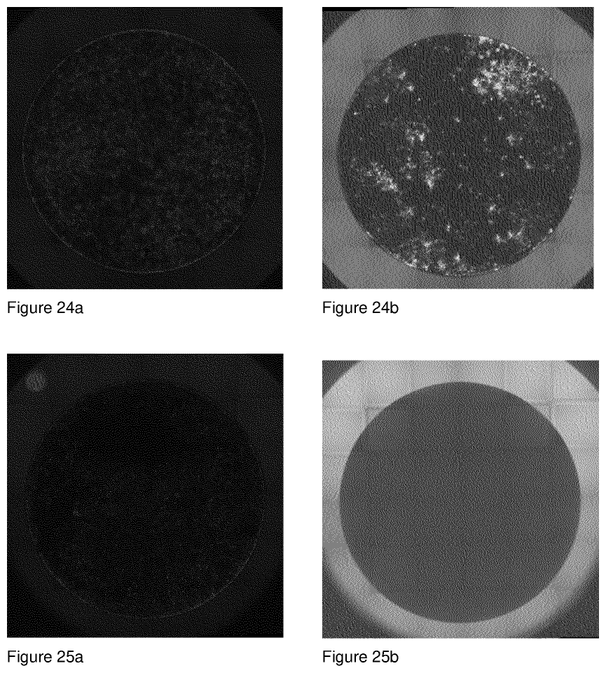

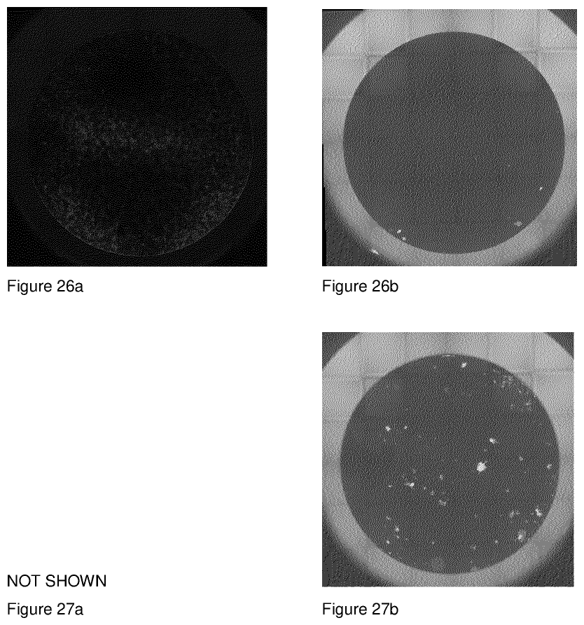

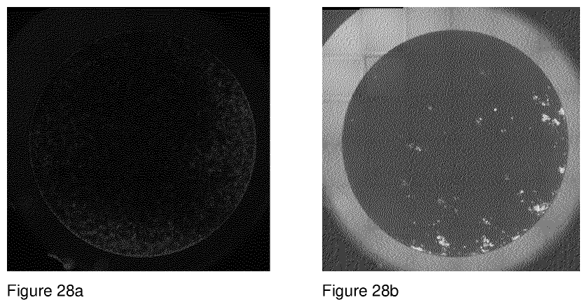

TABLE 1

| | 1 | 2 | 3 | 4 | 5 |
|---|---|---|---|---|---|
| A (FIGS. 6-10) | 50 nM | 5 nM | 0.5 nM | 5 nM | 0.5 nM |
| B (FIGS. 11-14) | 50 nM | 5 nM | 0.5 nM | 0.5 nM | |
| C (FIGS. 15-18) | 50 nM | 5 nM | 0.5 nM | 0.5 nM | |
| D (FIGS. 19-21) | 50 nM | 5 nM | 0.5 nM | | |
| E (FIGS. 22-24) | 50 nM | 5 nM | 0.5 nM | | |
| F (FIGS. 25-28) | 50 nM | 5 nM | 0.5 nM | 0.5 nM | |

A=DMSO (control); B=Compound 240 (DidemninB); C═PLD; D=Compound 9; E=Compound 10; F=PLD

Fluorescent cells were observed 24 hpi. Photos were obtained using an automated system. Cells were fixed for 30 min with PFA 4%, washed with PBS, and cell nuclei were stained with DAPI 1:200 in PBS 20 min RT. Images in green show GFP tagged vial particles. Images in blue show DAPI stained nuclei.

For a short while, confluent cultures of Huh-7 were infected at a multiplicity of infection (MOI) of 0.01 pfu/cell, with a viral inoculum of 3×10⁷ pfu/ml and after 8 hours, plitidepsin was added at concentrations ranging from 0.5 nM to 50 μM. The cultures with plitidepsin were incubated for 48 hours and then viral viability was measured by fluorescence. The results obtained showed an antiviral effect induced by plitidepsin at concentrations as low as 0.5 nM (0.555 pg/1), much lower than those reported with other antivirals.

It is shown that compounds of the invention are effective antiviral agents across a range of tested concentrations, whilst retaining cell viability.

### Example 3

A multicenter, randomized, parallel and proof of concept study was undertaken to evaluate the safety profile of three doses of Plitidepsin in patients with COVID-19 requiring hospitalization. Study details are available through ClinicalTrials.gov Identifier: NCT04382066.

The primary objective of the study was to determine the safety and toxicological profile of plitidepsin at each dose level administered according to the proposed administration scheme in patients admitted for COVID-19.

The secondary objectives were to assess the efficacy of plitidepsin in patients with COVID-19 at the proposed dose levels by reference to: change in SARS-CoV-2 viral load from baseline; time until negative detection of SARS-CoV-2 by PCR; cumulative incidence of disease severity (evaluation based on: mortality; need for invasive mechanical ventilation and/or ICU admission; need for non-invasive mechanical ventilation; need for oxygen therapy) and selection of the recommended dose levels of plitidepsin for a phase II/III efficacy study.

Patients included in the study were randomised in a 1:1:1 ratio to receive:
Arm A) 1.5 mg of plitidepsin administered as a 1.5-hour infusion, once a day for 3 consecutive days (total dose 4.5 mg).
Arm B) 2.0 mg of plitidepsin administered as a 1.5-hour infusion, once a day for 3 consecutive days (total dose 6.0 mg).
Arm C) 2.5 mg of plitidepsin administered as a 1.5-hour infusion, once a day for 3 consecutive days (total dose 7.5 mg).

All patients received the following prophylactic medications 20-30 minutes before the infusion of plitidepsin:
Diphenhydramine hydrochloride 25 mg iv or equivalent.
Ranitidine 50 mg iv or equivalent.
Dexamethasone 6.6 mg intravenous.
Ondansetron 8 mg i.v. in slow infusion of 15 minutes or equivalent.

Patients included in the study received treatment for 3 days.

Plitidepsin is supplied as a powder for concentrate for solution for infusion at a concentration of 2 mg/vial. Before use, the vials are reconstituted with 4 ml of reconstitution solution to obtain a colourless to slightly yellowish solution containing 0.5 mg/ml of plitidepsin, 25 mg/ml of mannitol, 0.15 ml/ml of macrogolglycerol ricinoleate oil, 0.15 ml/ml of ethanol and 0.70 ml/ml of water for injection. An additional dilution should be made in any suitable intravenous solution prior to infusion.

Plitidepsin 2 mg is supplied in a Type I clear glass vial with a bromobutyl rubber stopper covered with an aluminium seal. Each vial contains 2 mg of plitidepsin.

The solvent for the reconstitution of macrogolglycerol ricinoleate (polyoxyl 35 castor oil)/absolute ethanol/water for injection, 15%/15%/70% (v/v/v) is supplied in a Type I colourless glass vial. The ampoules have a volume of 4 ml.

Plitidepsin will be labelled with the study protocol code, the batch number, the content, the expiry date, the storage conditions, the name of the investigator and the sponsor. The study drug will be labelled in accordance with Annex 13 of the European Good Manufacturing Practices. Plitidepsin should be stored between 2° C. and 8° C. and the vials should be kept in the outer carton to protect them from light. The drug in these conditions is stable for 60 months.

After reconstitution of the 2 mg plitidepsin vial with 4 ml of the solution for reconstitution of macrogolglycerol ricinoleate/ethanol/water for injection, the reconstituted solution should be diluted and used immediately after preparation. If not used immediately, storage times and conditions until use are the responsibility of the user. The reconstituted concentrated solution of the drug product has been shown to be physically, chemically and microbiologically stable for 24 hours under refrigerated conditions (5° C.±3° C.) and for 6 hours when stored in the original vial under indoor light at room temperature. If storage is required before administration, solutions should be stored refrigerated and protected from light and should be used within 24 hours after reconstitution.

Plasma Concentration

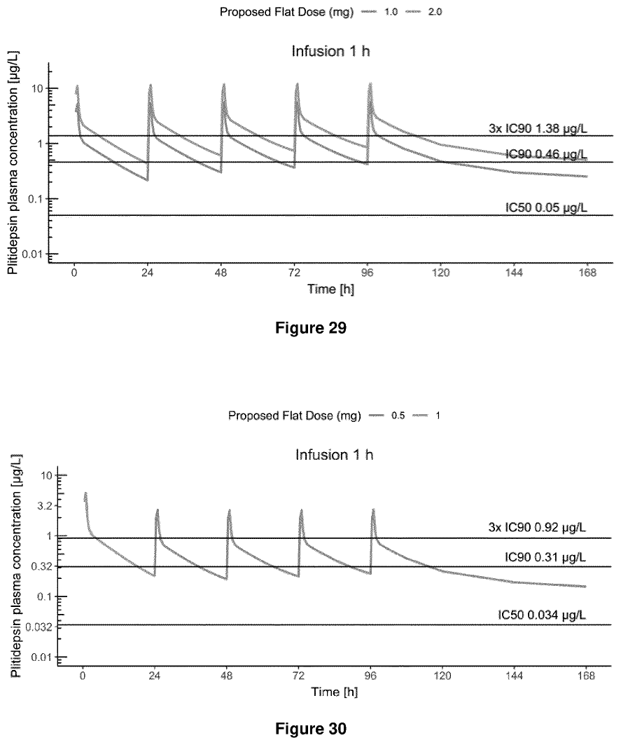

FIG. 29 illustrates the simulation of the total plasma plitidepsin concentration profiles vs. time after a daily dose (D1-D5) of 1.0 mg and 2.0 mg. The horizontal black lines represent the total plasma concentrations associated with the concentrations in lung equivalent to IC50, IC90 and 3×IC90 in vitro. With both dose levels (1.0 mg and 2.0 mg), plasma concentrations above IC50 would be obtained throughout the treatment period, and would remain above IC90 during most of the administration interval. Accumulation after five repeated administrations is minimal.

A further dosage regimen is 1.5 mg daily for 5 days. A further regimen is illustrated in FIG. 30 which simulates plitidepsin total plasma concentrations associated to an initial flat dose of 1 mg (Day 1) given as a 1-h i.v. infusion, followed by daily doses of 0.5 mg (D2-D5). With this dose regimen, plitidepsin plasma concentrations are above the IC50 during the entire treatment period, and remains above IC90 during 18 and 14 hours, after 1 mg and 0.5 mg dose infusion, respectively. Notably, minimal accumulation after repeated administration is foreseen. This regimen provides a loading dose of 1 mg of plitidepsin given as 1-h i.v. infusion on the first day of treatment, followed by a maintenance dose of 0.5 mg once daily for 4 days.

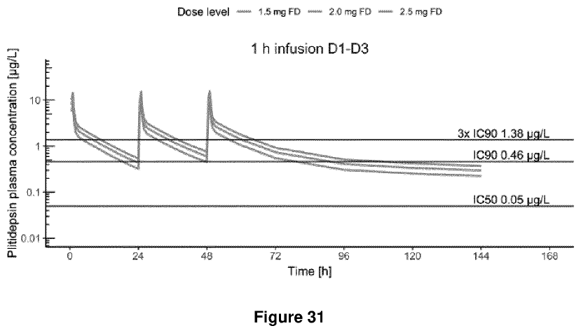

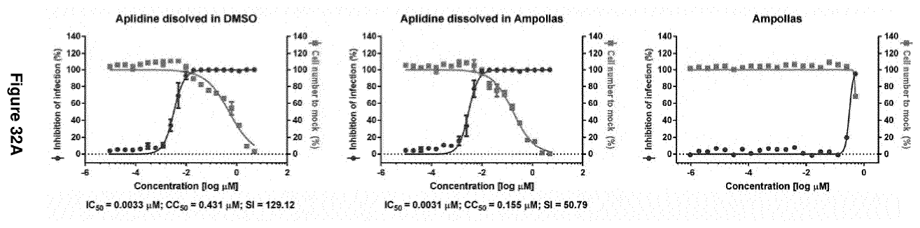

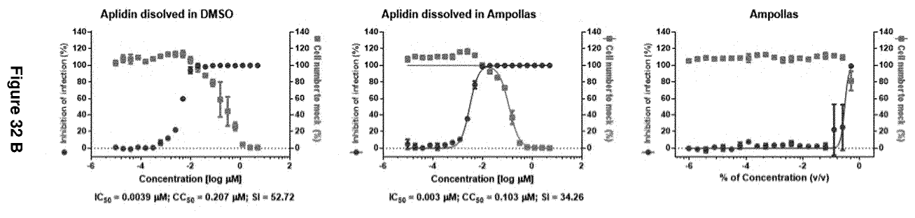

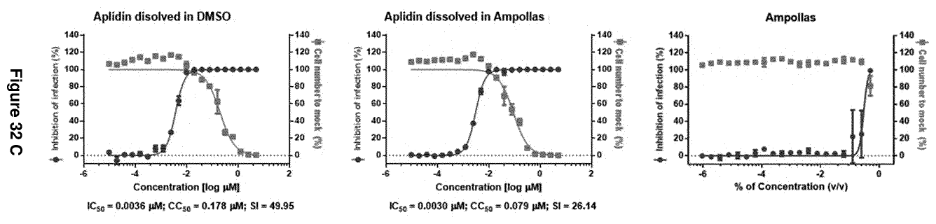

FIG. 31 illustrates the simulation of the total plasma plitidepsin concentration profiles vs. time after a daily dose (D1-D3) of 1.5 mg, 2.0 mg and 2.5 mg. The horizontal black lines represent the total plasma concentrations associated with concentrations in lungs equivalent to IC50, IC90 and 3×IC90 in vitro. With all three dosage levels (1.5 mg, 2.0 mg and 2.5 mg), plasma concentrations above IC50 would be obtained throughout the treatment period and would remain above IC90 during most of the administration interval. Accumulation after three repeated administrations is minimal.

Interim Results

To date, data is available for nine patients. PLD was administered as a 90 minute IV infusion daily for 3 consecutive days (day 1-3) with viral load assessed by PCR at baseline, day 4, day 7 and day 15 and day 31.

Patient 1—50 year old male, bilateral pneumonia. Received PLD 1.5 mg×3. PCR COVID 19 test: POSITIVE at baseline, converted to NEGATIVE (no viral load) by day 4. Acute clinical improvement. Hospital discharge by day 7. As such, PLD 1.5 mg×3 removed viral load by day 4. PLD achieved an acute clinical improvement, including removing all viral burden and treating bilateral pneumonia to enable hospital discharge by day 7.

Patient 2: 40 year old male, bilateral pneumonia. Received PLD 1.5 mg×3. By day six, lack of improvement and cross over to Remdesivir+TOL+Corticoids+Opiates. PCR converted to negative by day 15, Hospital discharge by Day 19.

Patient 3: 53 year old male, bilateral pneumonia. Received PLD 1.5 mg×3. PLD prevented clinical deterioration. Hospital discharge by day 10, PCR converted to negative by day 31.

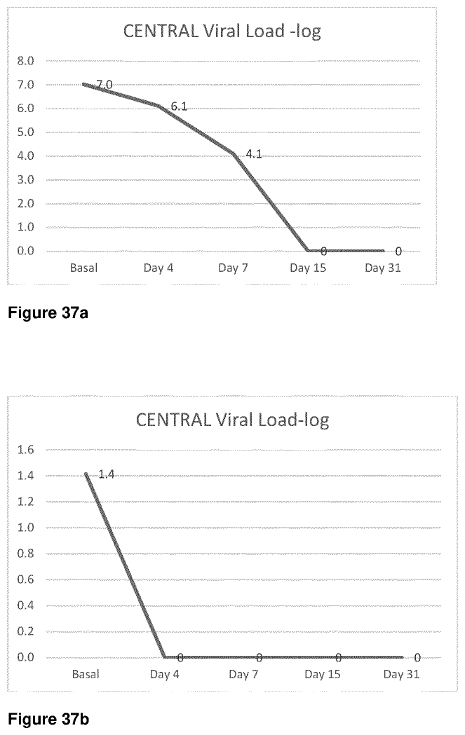

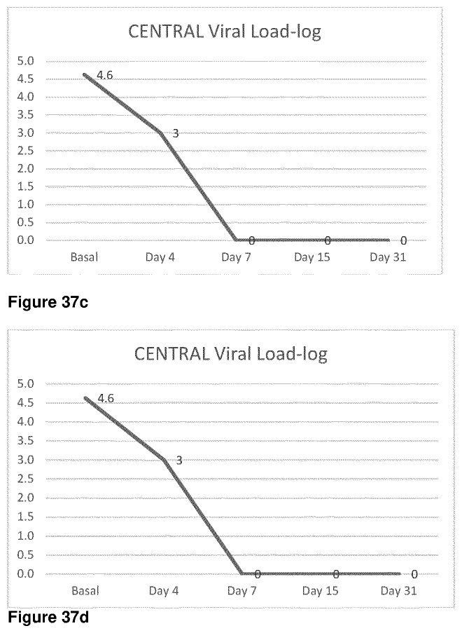

Patient 4: 42 year old male, bilateral pneumonia. Received PLD 2.0 mg×3. Corticoid therapy required. PCR COVID 19 test: POSITIVE at baseline, and still positive at day 7. By day 15 the patient was PCR negative, as shown in FIG. 37a. Patient recovered sufficiently for hospital discharge by day 10.

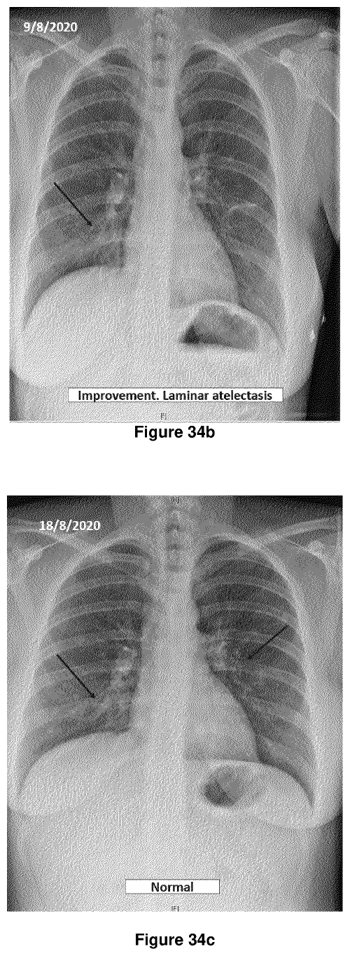

Patient 5: 33 year old female, bilateral pneumonia at entry. Received PLD 1.5 mg×3. PCR COVID 19 test: POSITIVE at baseline, converted to NEGATIVE (no viral load) by day 4 as shown in FIG. 37b. Bilateral pneumonia resolved by day 6 (normal Rx Lung). Major clinical improvement. Hospital discharge by day 8. X-rays showing pneumonia resolution shown in FIG. 34a-c. Bilateral pneumonia is evident in FIG. 34a. After treatment with PLD, improvement was seen on day 6. Laminar atelectasis is evidenced FIG. 34b. A follow up x-ray on day 15 showed return to normal FIG. 34c. PLD 1.5 mg×3 removed viral load by day 4. PLD achieved major clinical improvement, including removing all viral burden and treating bilateral pneumonia to enable hospital discharge by day 8.

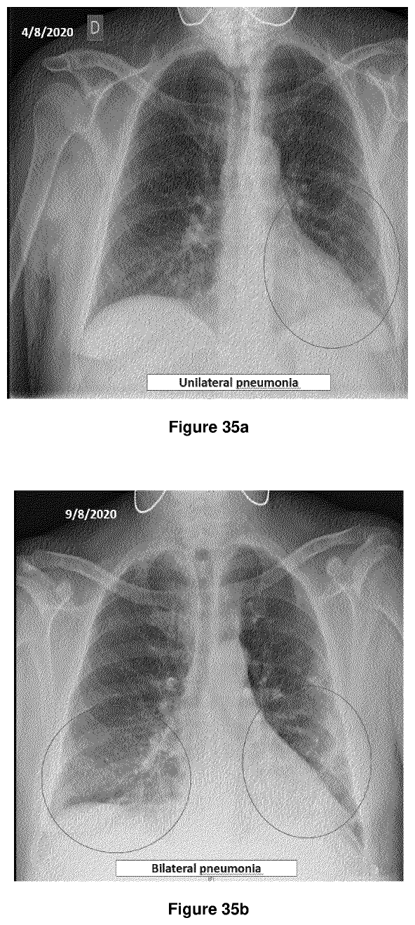

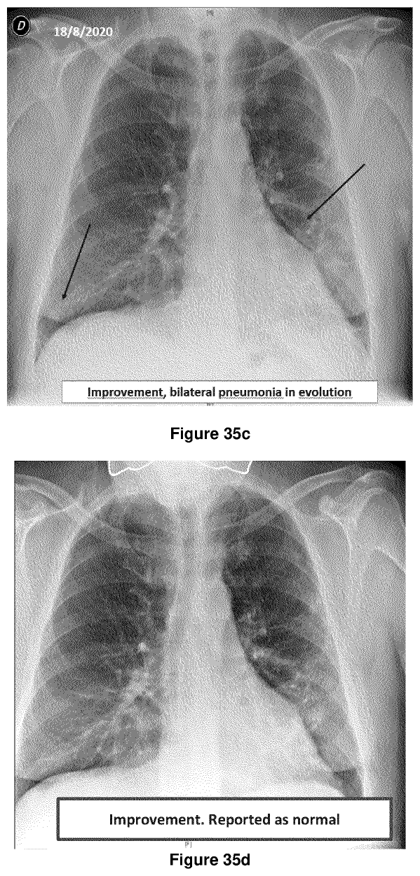

Patient 6: 69 year old female, highly symptomatic COPD. Unilateral pneumonia on entry. Received PLD 1.5 mg×3. PCR COVID 19 test: POSITIVE at baseline, converted to NEGATIVE (no viral load) by day 7 as shown in FIG. 37c. Major clinical improvement seen. Patient discharged by day 8. X-rays showing pneumonia progression shown in FIG. 35a-c. Unilateral pneumonia is evident in FIG. 35a which progressed to bilateral pneumonia in FIG. 35b. In FIG. 35c, improvement is seen. PLD achieved major clinical improvement, including removing all viral burden and treating pneumonia as shown in FIG. 35d to enable hospital discharge by day 8.

Patient 7: 39 year old female, pulmonary infiltrates. Received PLD 2.0 mg×3. PCR COVID 19 test: POSITIVE at baseline, converted to NEGATIVE (no viral load) by day 7 as shown in FIG. 37d. Following treatment with PLD, major clinical improvement. Hospital discharge by day 8.

Patient 8: 32 year old male. Received PLD 1.5 mg×3. Not evaluable for efficacy, hospital discharge by day 4.

Patient 9: 34 year old male. Received PLD 2.0 mg×3. PCR COVID 19 test: POSITIVE at baseline and still positive at day 7. However, major clinical improvement and hospital discharge by day 8.

C-Reactive Protein Tests

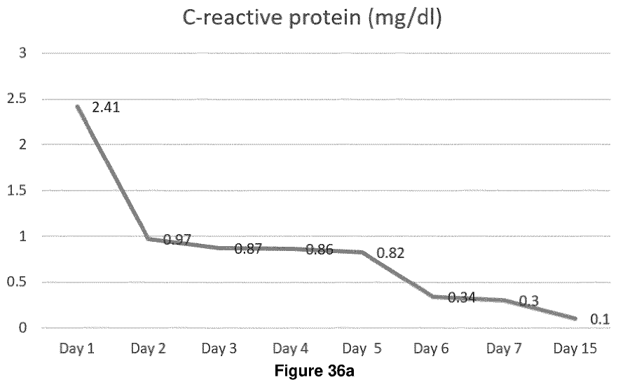

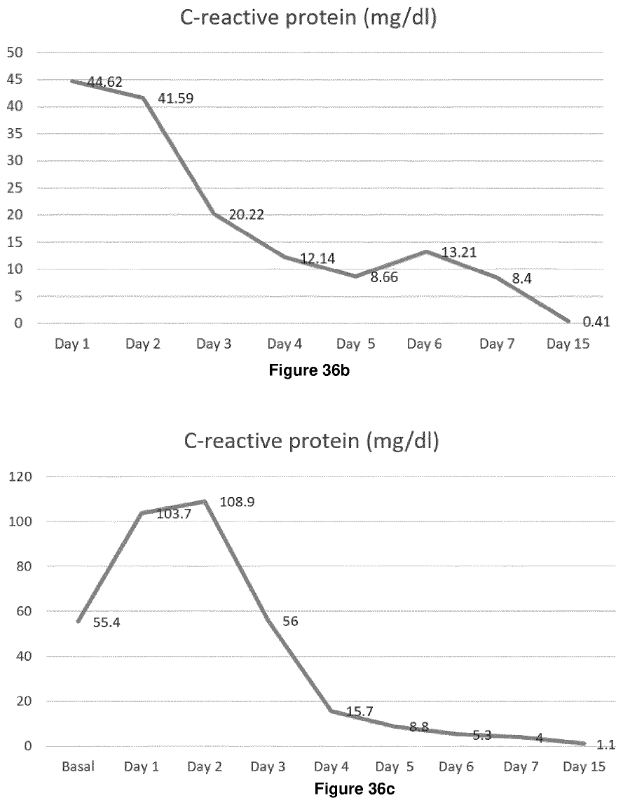

The effect of PLD on inflammatory cytokines was also measured for patients 5, 7 and 9 and the results of C-reactive protein tests are shown in FIG. 36. With patient 5 (FIG. 36a), following administration of PLD, an acute fall is seen by day 2. With patients 7 (FIG. 36b) and 9 (FIG. 36c), following administration of PLD, an acute fall is seen by day 3. These data demonstrate anti-inflammatory properties of PLD.

Discussion

The preliminary results of the PLD clinical trial on COVID-19 patients further demonstrate the remarkable properties of PLD in the treatment of SARS-CoV-2 infection and COVID-19.

6 out of 8 evaluable patients demonstrated SARS-CoV-2 PCR conversions to negative, with a median time for PCR conversion of 7 days (4-31). The antiviral properties of PLD on CoV infection, namely SARS-CoV-2, is clinically demonstrated with total removal of viral burden.

Remarkably, of the 9 patients currently tested, due to administration of PLD, none of the patients required mechanical ventilation or ICU admission, and there were no deaths on study. PDL induced disease control and major clinical improvements in 6 out of 8 evaluable patients with only 2 out of 8 requiring post PLD specific anti COVID 19 therapy. The median time to hospital discharge was 8 days (7-19). These results demonstrate that PLD is an effective therapy for SARS-CoV-2 infection and COVID-19, and pneumonia caused by SARS-CoV-2 infection.

Ad hoc analysis of the first cohort at 1.5 mg and 2 mg confirmed that PLD at the doses administered was active, achieved acute improvement and complete disease control. The complete removal of viral burden was noted in the vast majority of patients.

Results

Upon completion of the study, 45 patients hospitalised for COVID 19 were randomised to treatment with plitidepsin at doses of 1.5, 2.0, and 2.5 mg daily for 3 days. Treatment was well tolerated in all 3 dose cohorts. Treatment outcomes, assessed by hospital discharge rate, were driven by disease severity and viral load at baseline. Across dose cohorts, 100% (9/9) patients with mild disease, 82% (23/28) with moderate disease, and 57% (4/7) with severe disease were discharged by Day 15. Across dose cohorts, median viral load at baseline was 6.2 (0 to 10.6) log₁₀ copies/mL and a mean reduction in viral load of −3.1 log₁₀ copies/mL was achieved by Day 7 and 4.5 log₁₀ copies/mL by Day 15.

The study was a Phase 1, multicentre, open-label study in which 45 patients hospitalised for management of COVID-19 were randomised into 3 dose groups, comprising 1.5, 2.0, and 2.5 mg plitidepsin administered as a 1.5-hour IV infusion once a day for 3 consecutive days. The primary objective of this study was to determine the safety and toxicological profile at each dose level, based on (1) frequency of Grade ≥3 treatment-emergent adverse events (TEAE) at Days 3, 7, 15, and 31 using National Cancer Institute (NCI) Common Terminology Criteria for Adverse Events (CTCAE) version 5.0 criteria; (2) percentage of patients unable to complete treatment and reasons; (3) percentage of patients with TEAEs and SAEs at Days 3, 7, 15, and 31; (4) change from baseline haematologic and non-haematologic parameters on Days 3, 7, 15, and 31; and (5) percentage of patients with ECG abnormalities on Days 2, 3, 4, 5, 6, 7, 15, and 31. A secondary objective was to select a recommended dose for a pivotal study.

Findings for protocol-specified safety endpoints are summarised as follows:
Grade ≥3 AEs: Only 31%(14/45) of patients experienced Grade ≥3 AEs and only 2 patients experienced a treatment-related Grade ≥3 AE: 1 case each of anaphylactic reaction during the first plitidepsin infusion that resulted in treatment discontinuation and a case of diarrhoea that had no impact on plitidepsin treatment. Consistent with Grade ≥3 AEs being predominantly due to COVID-19 infection, the prevalence of Grade ≥3 AEs largely reflected the percentage of patients with severe disease in each cohort, with the highest prevalence in the 2.5-mg cohort (26.7% with severe disease, 40.0/Grade ≥3 AEs), a lower percentage in the 1.5-mg cohort (13.3% with severe disease, 33.3% Grade ≥3 AEs), and the lowest percentage in the 2.0-mg cohort (6.7% with severe disease, 20.0% Grade ≥3AEs). None of the events of special interest occurred, with the exception of 1 case of Grade ≥3 ALT elevation.
Patients unable to complete treatment: Only 1 patient was unable to complete plitidepsin treatment.
Patients with SAEs: A total of 10 patients experienced SAEs, including 6 dosed at 1.5 mg, 1 dosed at 2.0 mg, and 3 dosed at 2.5 mg. With the exception of 1 case of anaphylactic reaction, all these events were related to COVID-19 infection.
Patients with AEs: No dose-related trends were noted for any reported AEs and the most commonly reported AEs (excluding infections) were consistent with the safety profile observed for patients with advanced haematologic malignancies and solid tumours receiving single-agent plitidepsin, including gastrointestinal disorders of constipation (29% vs. 18% for cancer and COVID-19 patients, respectively), diarrhoea (31% vs. 18%), nausea (64% vs. 42%), and vomiting (38% vs. 18%) and constitutional symptoms of asthenia/fatigue (83% vs. 13%) and pyrexia (28% vs. 47%).
Changes in laboratory parameters: Although about half of patients (51%) showed ≥1 grade change in haematology parameters and most patients (89%) showed ≥1 grade change in chemistry parameters on study, few patients showed >1 grade shift. For haematology parameters, 6 patients had lymphopenia worsen by 2 to 3 grades and 2 had neutropenia worsen by 2 grades. For chemistry parameters, 5 patients had ALT increase by 2 to 3 grades, 2 patients had AST increase by 2 to 3 grades, 2 patients had GGT increase by 2 to 3 grades, and 1 patient had a 2 grade increase in CPK.

Based on these findings, it is concluded that plitidepsin treatment was well tolerated and it was not possible to detect a difference in safety between the 3 doses studied.

Efficacy in COVID-19 Patients

A total of 44 patients were evaluable for efficacy; 1 patient in the 1.5-mg cohort who experienced an anaphylactic reaction during the first infusion of plitidepsin had treatment discontinued and was not considered evaluable for efficacy. Results for protocol-specified efficacy endpoints showed equivalent results across the 3 dose cohorts (Table 2). Treatment outcomes were driven by baseline disease severity and viral load. Consistent with these findings, a recent study showed that SARS-CoV-2 viral load is associated with increased disease severity and mortality. Across dose cohorts, 100% (9/9) patients with mild disease were discharged by Day 15, compared to 82% (23/28) with moderate disease and 57% (4/7) with severe disease.

TABLE 2
Summary of Protocol-specified Efficacy Endpoints in Study APLICOV-PC

| | Dose Cohort | | | | | | | |
|---|---|---|---|---|---|---|---|---|
| | 1.5 mg | | 2.0 mg | | 2.5 mg | | Total | |
| Endpoint | N = 14^{A} | | N = 15 | | N = 15 | | N = 44 | |
| Patients discharged from hospital by | n (%) | | | | | | | |
| Day 8 | 6 | (42.9) | 9 | (60.0) | 10 | (66.7) | 25 | (56.8) |
| Day 15 | 11 | (78.6) | 14 | (93.3) | 11 | (73.3) | 36 | (81.8) |
| Day 31 | 13 | (92.9) | 14 | (93.3) | 13 | (86.7) | 40 | (90.9) |
| Mortality from Day 1 to | | | | | | | | |
| Day 7 | 0 | | 0 | | 0 | | 0 | |
| Day 15 | 0 | | 0 | | 0 | | 0 | |
| Day 31 | 1 | (7.1) | 0 | | 1 | (6.7) | 2 | (4.5) |
| Patients requiring invasive mechanical | | | | | | | | |
| ventilation and/or ICU admission from | | | | | | | | |
| Day 1 to | | | | | | | | |
| Day 7 | 2 | (14.3) | 1 | (6.7) | 2 | (13.3) | 5 | (11.4) |
| Day 15 | 2 | (14.3) | 1 | (6.7) | 3 | (20.0) | 6 | (13.6) |
| Day 31 | 2 | (14.3) | 1 | (6.7) | 3 | (20.0) | 6 | (13.6) |
| Patients requiring noninvasive | | | | | | | | |
| mechanical ventilation from Day 1 to | | | | | | | | |
| Day 7 | 3 | (21.4) | 0 | | 1 | (6.7) | 4 | (9.1) |
| Day 15 | 3 | (21.4) | 0 | | 2 | (13.3) | 5 | (11.4) |
| Day 31 | 3 | (21.4) | 1 | (6.7) | 2 | (13.3) | 6 | (13.6) |
| Patients requiring ICU admission from | | | | | | | | |
| Day 1 to | | | | | | | | |
| Day 7 | 2 | (14.3) | 1 | (6.7) | 2 | (13.3) | 5 | (11.4) |
| Day 15 | 2 | (14.3) | 1 | (6.7) | 3 | (20.0) | 6 | (13.6) |
| Day 31 | 2 | (14.3) | 1 | (6.7) | 3 | (20.0) | 6 | (13.6) |
| Patients requiring oxygen therapy at | | | | | | | | |
| Day 7 | 10 | (71.4) | 10 | (66.7) | 11 | (73.3) | 31 | (70.5) |
| Day 15 | 4 | (28.6) | 1 | (6.7) | 4 | (26.7) | 9 | (20.5) |

| | | | | | | | |
|---|---|---|---|---|---|---|---|
| Day 31 | 0 | 2 | (13.3) | 1 | (6.7) | 3 | (6.8) |

| Mean change in viral load from baseline | log₁₀ copies/mL | | | |
|---|---|---|---|---|
| to^{B} | | | | |
| Day 4 | −1.58 | −1.92 | −2.16 | −1.90 |
| Day 7 | −3.39 | −2.69 | −3.21 | −3.07 |
| Day 15 | −5.47 | −3.62 | −4.40 | −4.48 |
| Day 31 | −6.06 | −4.71 | −5.20 | −5.27 |
| Mean time from baseline until | 7 | 13 | 12 | 11 |
| undetectable viral load, days | | | | |

Abbreviations: ICU = intensive care unit
^{A}Patient who experienced an anaphylactic reaction during the first plitidepsin infusion
had treatment discontinued and was not considered evaluable for efficacy
^{B}Results based on 39 patients: 1 patient missed baseline viral load assessment and 4 patients had baseline viral load below limit of quantitation despite having positive polymerase chain reaction test within 48 hours prior to enrolment

Benefit-Risk Considerations

In the APLICOV-PC study, most patients (84%) had mild to moderate disease and 82% of patients were discharged by Day 15. As progressive deterioration of respiratory function and development of cytokine release syndrome typically occur at a mean of 10 days from onset of symptoms, the endpoint of hospital discharge rate at Day 15 is considered to reflect successful amelioration of life-threatening complications.

Post-hoc analyses showed that treatment response, assessed by hospital discharge rate, was correlated with baseline disease severity and viral load. Across dose cohorts, 100% (9/9) of patients with mild disease were discharged by Day 15, compared to 82% (23/28) with moderate disease and 57% (4/7) with severe disease.

In the APLICOV-PC study, median baseline viral load was 6.1 log₁₀ copies/mL and by Day 15 a mean −4.2 log₁₀ reduction in viral load was observed. These results support a conclusion that plitidepsin reduces viral replication.

Considering the low rate of drug-related Grade >3 AEs and the high discharge rate at Day 15, along with an average 4.2-log₁₀ reduction in baseline viral load by Day 15 (−3.0-log reduction reported for those patients with moderate disease), demonstrates a positive benefit risk for plitidepsin for treatment of patients hospitalised for COVID 19 infection.

### Example 4

The activity of plitidepsin against SARS-CoV-2 was further confirmed in an in vitro assay using vero cells.

Virus and Cells

SARS-CoV-2 was obtained from Korea Centers for Disease Control and Prevention (KCDC). Vero cells were acquired from the American Type Culture Collection (ATCC CCL-81).

Dose-Response Curve (DRC) Analysis by Immunofluorescence

The compound was prepared with two-fold serial dilutions at 20-point concentrations with DMSO and Ampolla (Cremophor:Ethanol:Water (15:15:70)) respectively. 24 hours after cell seeding, the compound was treated in the cells with the top concentration at 5 uM. After an hour, plates were transferred into the BSL-3 containment facility for viral infection and SARS-CoV-2 was added at a multiplicity of infection (MOI) of 0.0125. The plates were incubated at 37° C. for 24 hours. The cells were fixed at 24 hpi with 4% paraformaldehyde (PFA) for permeabilization. Anti-SARS-CoV-2 Nucleocapsid (N) 1st antibody and 488-conjugated goat anti-rabbit IgG 2nd antibody were treated to the cells and Hoechst 33342 were treated to dye the cells for the analysis by immunofluorescence. The acquired images with Operetta (Perkin Elmer) were analyzed using in-house software to quantify cell numbers and infection ratios, and antiviral activity was normalized to positive (mock) and negative (0.5% DMSO) controls in each assay plate.

DRCs were fitted by sigmoidal dose-response models, with the following equation: Y=Bottom+(Top Bottom)/(1+(IC₅₀/X)Hillslope), using XLfit 4 Software or Prism7. IC₅₀ values were calculated from the normalized activity dataset-fitted curves. All IC₅ and CC₅₀ values were measured in duplicate, and the quality of each assay was controlled by Z′-factor and the coefficient of variation in percent (% CV).

Dose-response curves are shown in FIGS. 32A-C (three repeats). The blue squares represent inhibition of virus infection (%) and the red triangles represent cell viability (%). Means i SD were calculated from duplicate experiments. Plitidepsin was able to inhibit viral-induced cytopathic effects (squares) at concentrations where no cytotoxic effects of the drug were observed (circles) in all experiments. In this experiment, plitidepsin had an IC₅₀ of 0.0033-0.0039 μM versus a CC₅₀ of 0.178-0.431 μM giving an SI of 49.95-129.92.

### Example 5

The activity of plitidepsin against SARS-CoV-2 was further confirmed in a different in vitro assay using vero cells.

Cell Cultures

Vero E6 cells (ATCC CRL-1586) were cultured in Dulbecco's modified Eagle medium, (DMEM; Lonza) supplemented with 5% fetal calf serum (FCS; EuroClone), 100 U/mL penicillin, 100 μg/mL streptomycin, and 2 mM glutamine (all ThermoFisher Scientific).

Virus Isolation, Titration and Sequencing

SARS-CoV-2 virus was isolated from a nasopharyngeal swab collected from an 89-year-old male patient giving informed consent and treated with Betaferon and hydroxychloroquine for 2 days before sample collection. The swab was collected in 3 mL medium (Deltaswab VICUM) to reduce viscosity and stored at −80° C. until use. Vero E6 cells were cultured on a cell culture flask (25 cm²) at 1.5×10⁶ cells overnight prior to inoculation with 1 mL of the processed sample, for 1 h at 37° C. and 5% CO₂. Afterwards, 4 mL of 2% FCS-supplemented DMEM were supplied and cells were incubated for 48 h. Supernatant was harvested, centrifuged at 200×g for 10 min to remove cell debris and stored at −80° C. Cells were assessed daily for cytopathic effect and the supernatant was subjected to viral RNA extraction and specific RT-qPCR using the SARS-CoV-2 UpE, RdRp and N assays (Corman et al., 2020). The virus was propagated for two passages and a virus stock was prepared collecting the supernatant from Vero E6.

Viral RNA was extracted directly from the virus stock using the Indimag Pathogen kit (Indical Biosciences) and transcribed to cDNA using the PrimeScript™ RT reagent Kit (Takara using oligo-dT and random hexamers, according to the manufacturer's protocol. DNA library preparation was performed using SWIFT amplicon SARS-CoV-2 panel (Swift Biosciences). Sequencing ready libraries where then loaded onto Illumina MiSeq platform and a 300 bp paired-end sequencing kit. Sequence reads were quality filtered and adapter primer sequences were trimmed using trimmomatic. Amplification primer sequences were removed using cutadapt (Martin, 2011). Sequencing reads were then mapped against coronavirus reference (NC_045512.2) using bowtie2 tool (Langmead, B. and Salzberg, S, 2012). Consensus genomic sequence was called from the resulting alignment at a 18×1800×879 average coverage using samtools (Li et al., 2009). Genomic sequence was deposited at GISAID repository (http://gisaid.org) with accession ID EPI_ISL_510689.

Compound

Plitidepsin was used at a concentration ranging from 100 μM to 0.0512 nM at 1/5 serial dilutions, and also assayed from 10 μM to 0.5 nM at 1/3 dilutions.

Antiviral Activity

Increasing concentrations of Plitidepsin was added to Vero E6 cells together with 10^{1.8} TCID₅₀/mL of SARS-CoV-2, a concentration that achieves a 50% of cytopathic effect. Non-exposed cells were used as negative controls of infection. In order to detect any drug-associated cytotoxic effect, Vero E6 cells were equally cultured in the presence of increasing drug concentrations, but in the absence of virus. Cytopathic or cytotoxic effects of the virus or drugs were measured at 3 days post infection, using the CellTiter-Glo luminescent cell viability assay (Promega). Luminescence was measured in a Fluoroskan Ascent FL luminometer (ThermoFisher Scientific).

IC₅₀ Calculation and Statistical Analysis

Response curves were adjusted to a non-linear fit regression model, calculated with a four-parameter logistic curve with variable slope. Cells not exposed to the virus were used as negative controls of infection and set as 100% of viability, and used to normalize data and calculate the percentage of cytopathic effect. Statistical differences from 100% were assessed with a one sample t test. All analyses and figures were generated with the GraphPad Prism v8.0b Software.

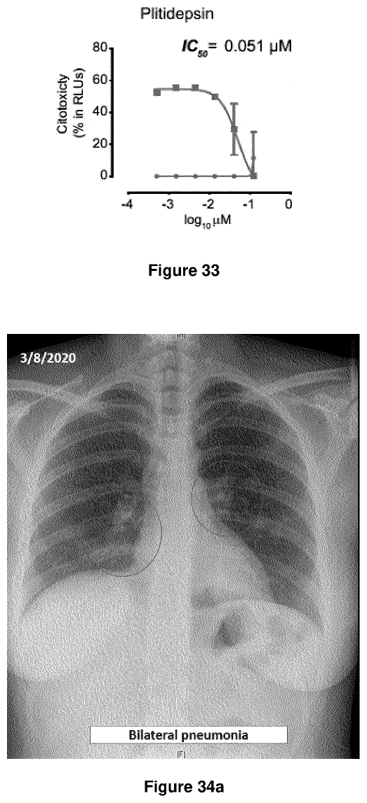

The cytopathic effect on Vero E6 cells exposed to a fixed concentration of SARS-CoV-2 in the presence of increasing concentrations of plitidepsin is shown in FIG. 33. Drug was used at a concentration ranging from 10 μM to 0.5 nM at 1/3 dilutions. Non-linear fit to a variable response curve from one representative experiment with two replicates is shown (squares). The particular IC₅₀ value of this experiment is indicated in the figure. Cytotoxic effect on Vero E6 cells exposed to increasing concentrations of plitidepsin in the absence of virus is also shown (circles).

A constant concentration of SARS-CoV-2 was mixed with increasing concentrations of plitidepsin and added to Vero E6 cells. To control for drug-induced cytotoxicity, Vero E6 cells were also cultures with increasing concentrations of plitidepsin in the absence of SARS-CoV-2.

Plitidepsin was able to inhibit viral-induced cytopathic effects (red squares) at concentrations where no cytotoxic effects of the drug were observed (grey circles). The mean IC₅₀ value and standard deviation of plitidepsin in two experiments with two replicates each was 0.06 i 0.02 μM.

### Example 6 A Phase 3, Multicentre, Randomised, Controlled Trial to Determine the Efficacy and Safety of Two Dose Levels of Plitidepsin Versus Control in Adult Patients Requiring Hospitalisation for Management of Moderate COVID 19 Infection

Indication: Treatment of patients hospitalised for management of moderate COVID 19 infection

Objectives:

Primary objective:
Compare plitidepsin 1.5 or 2.5 mg versus control on the percentage of patients who achieve complete recovery by Day 8 (±1), defined as (i) meeting categories 0 to 2 on the 11-point World Health Organization (WHO) Clinical Progression Scale below, (ii) having Barthel Index >90/100 at the time of discharge, and (iii) with no re-admission for COVID-19 infection through Day 31
   0: uninfected, no viral RNA detected
   1: asymptomatic, viral RNA detected
   2: symptomatic, independent
   3: symptomatic, assistance needed
   4: hospitalised, no oxygen therapy (if hospitalised for isolation only, record status as for ambulatory patient)
   5: hospitalised, oxygen by mask or nasal prongs
   6: hospitalised, oxygen by noninvasive ventilation (NIV) or high flow
   7: intubation and mechanical ventilation. pO₂/FIO₂≥150 or SpO₂/FIO₂≥200
   8: mechanical ventilation pO₂/FIO₂<150 (SpO₂/FIO₂<200) or vasopressors
   9: mechanical ventilation pO2/FIO₂<150 and vasopressors, dialysis, or extracorporeal membrane oxygenation (ECMO)
   10: dead

Key secondary objectives of this study are to compare plitidepsin 1.5 and 2.5 mg versus control on the following:
Time to complete recovery (in days), defined as the first day, from Day 1 through follow-up on Day 31, on which a patient (i) satisfies categories 0 to 2 on the 11-point WHO Clinical Progression Scale above, (ii) has Barthel Index >90/100 at the time of discharge, and (iii) has no subsequent re-admission for COVID-19 infection
Clinical status, as assessed by the 11-category WHO Clinical Progression Scale, at Day 8 (±1)

Other secondary objectives of this study are:
Safety and tolerability, based on treatment-emergent adverse events (TEAEs), Grade ≥3 TEAEs, serious adverse events (SAEs), and serious adverse reactions (SARs)
Compare the efficacy and safety/tolerability between plitidepsin arms (1.5 versus 2.5 mg) in case both arms are significantly better than the control arm on the primary endpoint
Compare the percentage of patients who achieve complete recovery by Day 8 (±1), as defined above, for the pooled plitidepsin arms versus control
Percentage of patients in each study arm requiring re-admission for COVID-19 infection
Clinical status in each study arm, as assessed by the 11-category WHO Clinical Progression Scale, at Days 4, 15, and 31
Duration of oxygen therapy (in days) for each study arm
Percentage of patients in each study arm requiring high-flow oxygen at Days 4, 8, 15, and 31
Percentage of patients in each study arm requiring noninvasive mechanical ventilation at Days 4, 8, 15, and 31
Percentage of patients in each study arm requiring invasive mechanical ventilation or ECMO at Days 4, 8, 15, and 31
Percentage of patients in each study arm requiring intensive care unit (ICU) admission at Days 4, 8, 15, and 31
Duration of hospitalisation in ICU for each study arm
Percentage of patients receiving subsequent antiviral therapies or immunomodulatory drugs at Days 4, 8, 15, and 31
Percentage of patients in each study arm with nosocomial infection
Mortality in each study arm at Days 4, 8, 15, and 31
Change in severe acute respiratory syndrome coronavirus 2 (SARS-CoV-2) viral load in each study arm from Day 1 before administration of study drug to Day 8 as measured by quantitative polymerase chain reaction (qPCR) from samples of oro-nasopharyngeal exudate
Percentage of patients in each study arm with undetectable SARS-CoV-2 viral load on Day 8, as measured by qPCR from samples of oro-nasopharyngeal exudate

Change in proinflammatory biomarkers (C-reactive protein [CRP], lactate dehydrogenase [LDH], ferritin, interleukin [IL]-6, IL-10, IL-10, and tumour necrosis factor alpha [TNFα]) in each study arm from baseline to Days 2, 3, 4, 8, 15, and 31

Methodology/Study Design:

This is a multicentre, open label, controlled Phase 3 study in which adults requiring hospital admission and 02 supplementation for management of moderate COVID 19 infection will be randomised in 1:1:1 to:
Plitidepsin 1.5 mg arm: Patients will receive plitidepsin 1.5 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).
Plitidepsin 2.5 mg arm: Patients will receive plitidepsin 2.5 mg/day IV combined with dexamethasone 6.6 mg/day IV on Days 1 to 3, followed by dexamethasone 6 mg/day PO/IV from Day 4 and up to Day 10 (as per physician judgement according to patient clinical condition and evolution).
Control arm: Patients will receive dexamethasone 6.6 mg/day IV on Days 1 to 3, followed by dexamethasone 6 mg/day PO/IV from Day 4 and up to Day 10 (as per physician judgement according to patient clinical condition and evolution). Additionally, in accordance with local treatment guidelines, patients may receive remdesivir 200 mg IV on Day 1 followed by 100 mg/day IV on Days 2 to 5.

Randomisation will be stratified for 2 factors:
Intention to administer remdesivir if the patient is randomised to the control arm (yes vs. no); and
Charlson Comorbidity Index13,14 (0-1 vs. >1)

From treatment initiation on Day 1, patients will be followed in the hospital for at least 4 days and then through Day 31 or resolution/stabilisation of TEAEs that occurred through Day 31. Patients discharged from the hospital prior to Day 8 will return to an out-patient clinic for assessments on Days 8 and 31 (Appendix 5).

An Independent Data Monitoring Committee (IDMC) will oversee study conduct (safety and primary endpoint), including analysis of summary safety data as per the trial requirements.

Diagnosis and main criteria for inclusion and exclusion:

The following are the inclusion criteria:
1. Signed informed consent obtained prior to initiation of any study specific procedures and study treatment
2. Laboratory confirmed SARS-CoV-2 infection as determined by qualitative polymerase chain reaction (PCR) by local laboratory from oro/nasopharyngeal exudate (or other respiratory specimen) collected no more than 48 hours prior to study treatment on Day 1
3. Admitted to hospital as clinically indicated for management of moderate SARS-CoV-2 (COVID 19) infection, defined by the following criteria:
   Positive PCR test for SARS-CoV-2
   Symptoms of moderate illness with COVID 19, which could include any symptoms of mild illness or shortness of breath with exertion
   Clinical signs suggestive of moderate illness with COVID 19 such as respiratory rate ≥20 breaths but <30 breaths per minute, SpO2>93% but <95% on room air at sea level, heart rate ≥90 but <125 beats per minute, and requiring 02 supplementation
   No clinical signs indicative of severe illness, which could include shortness of breath at rest or respiratory distress
4. Onset of COVID 19 symptoms no later than 6 days prior to initiation of study treatment on Day 1
5. Male or female aged ≥18 years
6. Adequate bone marrow, liver, kidney, and metabolic function, defined by the following tests performed at local laboratory:
   Absolute neutrophil count 21000/mm3 (1.0×109/L)
   Lymphocyte count ≥500/mm3 (0.5×109/L)
   Platelet count ≥100 000/mm3 (100×109/L)
   Haemoglobin >9.0 g/dL
   Alanine transaminase (ALT), aspartate transaminase (AST) ≤3× upper limit of normal (ULN)
   Serum bilirubin ≤1×ULN (≤3×ULN if documented Gilbert's syndrome)
   Calculated creatinine clearance ≥30 mL/min (Cockcroft and Gault formula)
   Creatine phosphokinase ≤2.5×ULN
7. Agree not to participate in another interventional clinical trial through Day 31
8. Females of reproductive capacity must have a negative serum pregnancy test by local laboratory at study enrolment and must be non-lactating
9. Females and males with partners of child bearing potential must use effective contraception while on study treatment and for 3 months after last dose of plitidepsin.

The following are the exclusion criteria:
1. Subjects with a pre-baseline (ie, in the prior month) impairment in general health condition for whatever reason except COVID 19, requiring either assistance for daily living activities or chronic oxygen therapy
2. Participating in another clinical trial for treatment of COVID 19 infection or patients previously enrolled in clinical trials and currently in follow up, or patients previously vaccinated for COVID 19
3. Evidence of respiratory failure at the time of randomisation, based on resource utilisation requiring at least 1 of the following: endotracheal intubation and mechanical ventilation, oxygen delivered by high-flow nasal cannula, noninvasive positive pressure ventilation, ECMO, or clinical diagnosis of respiratory failure (ie, clinical need for 1 of the preceding therapies, but preceding therapies not able to be administered in setting of resource limitation)
4. Patients clinically indicated for management of SARS-CoV-2 (COVID 19), with baseline disease severity rated as severe (if positive testing by standard RT PCR assay or equivalent test, symptoms suggestive of severe illness with COVID 19, which could include any symptom of moderate illness or shortness of breath at rest, or respiratory distress, clinical signs indicative of severe systemic illness with COVID 19, such as respiratory rate ≥30 per minute, heart rate ≥125 per minute, SpO2≤93% on room air at sea level, or PaO2/FiO2<300)
5. Patients receiving treatment with antivirals, IL 6 receptor inhibitor, corticosteroids, or immunomodulatory drugs for COVID 19 infection within 4 weeks before enrolment
6. History of live vaccination within the last 4 weeks prior to study enrolment; subjects must not receive live, attenuated influenza vaccine within 4 weeks before enrolment or at any time during the study
7. Patients receiving treatment with chloroquine or derivatives within 8 weeks before enrolment or during the study
8. Receiving treatment with strong cytochrome P450 3A4 (CYP3A4) inhibitors or inducers
9. Viral illness (other than COVID 19) requiring therapy, except for patients with treated and adequately controlled (undetectable) human immunodeficiency virus infection are eligible
10. QT interval corrected using Fridericia's formula prolongation >450 msec for males or >470 msec for females, based on triplicate electrocardiogram (ECG) at screening
11. Pre-existing neuropathies of any type Grade >2
12. Hypersensitivity to the active ingredient or any of the excipients (mannitol, macrogolglycerol hydroxystearate, and ethanol).
13. Females who are pregnant (negative serum pregnancy test required for all females of child bearing potential at screening) or breast feeding
14. Females and males with partners of child bearing potential (females who are not surgically sterile or postmenopausal defined as amenorrhoea for >12 months) who are not using at least 1 protocol-specified method of contraception
15. Any other clinically significant medical condition or laboratory abnormality that, in the opinion of the investigator, would jeopardise the safety of the patient or potentially impact patient compliance or the safety/efficacy observations in the study.

Test Products, Dose, and Mode of Administration:

Plitidepsin for injection is provided in vials containing 2 mg plitidepsin powder. For administration, vial contents are reconstituted by addition of 4 mL of solvent for plitidepsin to obtain a slightly yellowish solution containing 0.5 mg/mL plitidepsin with mannitol, macrogolglycerol hydroxystearate, and ethanol excipients. The required amount of plitidepsin reconstituted solution is added to an IV bag containing 0.9% sodium chloride injection or 5% glucose for injection and administered as an IV infusion over 60 minutes.

For prevention of plitidepsin related infusion reactions, all patients must receive the following medications 20 to 30 minutes prior to starting the plitidepsin infusion:
Ondansetron 8 mg IV (or equivalent; note: ondansetron is a prohibited medication for patients in the corrected QT interval [QTc] substudy during the QT evaluation [Days 1 to 3])
Diphenhydramine hydrochloride 25 mg IV (or equivalent)
Ranitidine 50 mg IV (or equivalent)
Dexamethasone 6.6 mg IV

Additionally, on Days 4 and 5 patients treated with plitidepsin must receive ondansetron 4 mg twice a day PO.

Reference Therapy, Dose, Dose Form, and Mode of Administration:

Dexamethasone: Patients on both plitidepsin and control arms will receive dexamethasone 6.6 mg/day IV on Days 1 to 3, followed by dexamethasone 6 mg/day PO/V from Day 4 and up to Day 10 (as per physician judgement according to patient clinical condition and evolution).

Remdesivir: Consistent with local treatment guidelines, patients randomised to the control arm may receive remdesivir 200 mg IV on Day 1 followed by 100 mg/day IV on Days 2 to 5.

Best Supportive Care (BSC): BSC consistent with National Institute of Health COVID 19 treatment guidelines (www.covid19treatmentguidelines.nih.gov) or other country guidelines will be provided to all study participants.

### Example 7 Open-Label, Randomized Phase II Study to Evaluate the Safety and Reduction of Viral Load of a Single-Line Treatment with Plitidepsin in Adult Patients with COVID-19 at Discharge from the Emergency Department

Indication: Treatment of patients with mild type COVID 19 infection.

Patients will be included in the study if presenting with acute clinical infection (onset of symptoms in the previous 5 days), in which the diagnosis of COVID-19 infection is reached through a diagnostic method that could be a positive antigen test or a positive PCR test.

The study comprises two arms:
arm A) single dose of 7.5 mg plitidepsin administered as a slow infusion of 90 minutes (±10 minutes) plus symptomatic treatment according to routine clinical practice in the participating centers.
arm B) symptomatic treatment according to usual clinical practice in the participating centers.

All patients receive the following prophylactic medications 20-30 minutes prior to plitidepsin infusion:
Diphenhydramine hydrochloride 25 mg i.v.,
Ranitidine 50 mg i.v.
Dexamethasone 6.6 mg intravenously.
Ondansetron 8 mg i.v. in slow infusion of 15 minutes.

Ondansetron 4 mg orally is given every 12 hours for 3 days after plitidepsin administration to relieve drug-induced nausea and vomiting. If plitidepsin is administered in the morning the patient receives the first dose of ondansetron in the afternoon.

The study will show that a single dose of plitidepsin administered to patients results in a reduction of viral load. This may be expressed as a replication cycle threshold (Ct) value greater than 30 (Ct>30), on day 6 after the administration. For example, the study will show that patients with COVID-19 infection who are to be discharged from the Emergency Department show a reduction in viral load on day 6 after discharge of emergencies expressed as a replication cycle threshold (Ct) value greater than 30 (Ct>30), when administered with a single dose of plitidepsin. This may be expressed as a reduction in SARS-CoV-2 viral load from baseline. This may be expressed as a reduction in the percentage of patients requiring hospitalisation following administration. This may be expressed as a reduction in the percentage of patients requiring invasive mechanical ventilation and/or admission to the ICU following administration. This may be expressed as a reduction of patients who develop sequelae related to persistent disease. This may be expressed as an increase in the percentage of patients with normalization of analytical parameters chosen as poor prognosis criteria (including, for example, lymphopenia, LDH, D-dimer or PCR). This may be expressed as an increase in the percentage of patients with normalization of clinical criteria (disappearance of symptoms), including, for example: headache, fever, cough, fatigue, dyspnea (shortness of breath), arthromyalgia or diarrhea.

The study will show that single-dose treatment with plitidepsin can eliminate the SARS-CoV-2 viral load in the patient on day 6, which, according to several studies, leads to clinical improvement, and therefore a decrease in complications, understood as hospitalization, ICU and death. In addition to improving the prognosis of patients in the short term, the decrease in viral load is believed to be key to two other objectives. Firstly, reducing the infectivity of asymptomatic or not very symptomatic patients with high viral loads (TC<25), known as supercontagators. Secondly, decreasing viral load can be decisive to avoid long-term complications known as COVID persistent or long COVID.

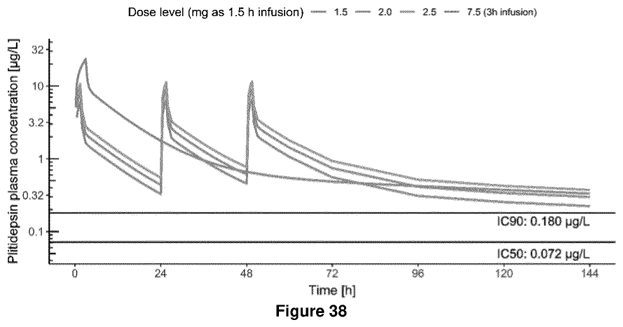

A validated plitidepsin population pharmacokinetic model (Nalda-Molina R, et al. Population pharmacokinetics meta-analysis of plitidepsin (Aplidin) in cancer subjects. Cancer Chemother Pharmacol. 2009 June; 64(1):97-108. doi: 10.1007/s00280-008-0841-4) was used to confirm total plasma concentration will reach the estimated lung target concentrations. FIG. 38 shows the results and it can be seen that plasma concentrations above IC50 and IC90 can be obtained for more than 6 days.

### REFERENCES

Almazan, F., Galan, C., Enjuanes, L., 2004. The nucleoprotein is required for efficient coronavirus genome replication. J. Virol. 78, 12683-12688. 10 Baric et al., 1988
Baric, R. S., Nelson, G. W., Fleming, J. O., Deans, R. J., Keck, J. G., Casteel, N., Stohlman, S. A. Interactions between coronavirus nucleocapsid protein and viral RNAs: implications for viral transcription. J. Virol. 1988, 62, 4280-4287
Escors, D., Camafeita, E., Ortego, J., Laude, H., Enjuanes, L. Organization of two transmissible gastroenteritis coronavirus membrane protein topologies within the virion and core. J. Virol. 2001, 75, 12228-12240.
Fang, S. G.; Shen, H.; Wang, J.; Tay, F. P.; Liu, D. X. Proteolytic processing of polyproteins 1a and 1ab between non-structural proteins 10 and 11/12 of Coronavirus infectious bronchitis virus is dispensable for viral replication in cultured cells. Virology 2008, 379, 175-180.
Graham, R. L.; Donaldson, E. F.; Baric, R. S. A decade after SARS: Strategies for controlling emerging coronaviruses. Nat. Rev. Microbiol. 2013, 11, 836-848.
Lai, M. M.; Cavanagh, D. The molecular biology of coronaviruses. Adv. Virus Res. 1997, 48, 1-100.
Losada A, Berlanga J J, Molina-Guijarro J M, Jiménez-Ruiz A, Gaggo F, Avilés P, de Haro C, Martínez-Leal J F., Generation of endoplasmic reticulum stress and inhibition of autophagy by plitidepsin induces proteotoxic apoptosis in cancer cells. Biochem Pharmacol. 2020 February; 172:113744
Losada A, Muñoz-Alonso M J, García C, Sánchez-Murcia P A, Martínez-Leal J F, Domínguez J M, Lillo M P, Gago F, Galmarini C M., Translation Elongation Factor eEF1A2 is a Novel Anticancer Target for the Marine Natural Product Plitidepsin. Sci Rep. 2016 7; 6:35100
Losada A, Muiloz-Alonso M J, Martínez-Díez M, Gago F, Domínguez J M, Martínez-Leal J F, Galmarini C M., Binding of eEF1A2 to the RNA-dependent protein kinase PKR modulates its activity and promotes tumour cell survival. Br J Cancer. 2018; 119(11):1410-1420
M. D. Vera and M. M. Joullié, Natural products as probes of cell biology: 20 years of didemnin research Med. Res. Rev., 2002, 22, 102
Pyrc, K.; Berkhout, B.; van der Hoek, L. The novel human coronaviruses NL63 and HKU1. J. Virol. 2007, 81, 3051-3057.
Rabouw H H, Langereis M A, Knaap R C, Dalebout T J, Canton J, Sola I, Enjuanes L, Bredenbeek P J, Kikkert M, de Groot R J, van Kuppeveld F J., Middle East Respiratory Coronavirus Accessory Protein 4a Inhibits PKR-Mediated Antiviral Stress Responses. PLoS Pathog. 2016, 26; 12(10):e1005982.
Remington s Pharmaceutical Sciences, 17th Ed., Mack Publishing Company, Easton, Pa., 1995
Risco, C., Anton, I. M., Enjuanes, L., Carrascosa, J. L. The transmissible gastroenteritis coronavirus contains a spherical core shell consisting of M and N proteins. J. Virol. 1996, 70, 4773-4777
Snijder, E. J.; Bredenbeek, P. J.; Dobbe, J. C.; Thiel, V.; Ziebuhr, J.; Poon, L. L.; Guan, Y.; Rozanov, M.; Spaan, W. J.; Gorbalenya, A. E. Unique and conserved features of genome and proteome of SARS-coronavirus, an early split-off from the coronavirus group 2 lineage. J. Mol. Biol. 2003, 331, 991-1004
Stark G. R., Kerr, I. M., Williams, B. R., Silverman, R. H., and Schreiber, R. D., How cells respond to interferons. Annu. Rev. Biochem. 1998, 67: 227-264.
Stohlman, S. A., Baric, R. S., Nelson, G. N., Soe, L. H., Welter, L. M., Deans, R. J. Specific interaction between coronavirus leader RNA and nucleocapsid protein. J. Virol. 1988, 62, 4288-4295
Sturman, L. S., Holmes, K. V., Behnke, J. Isolation of coronavirus envelope glycoproteins and interaction with the viral nucleocapsid. J. Virol. 1980, 33, 449-462.
Williams B. R. PKR; a sentinel kinase for cellular stress. Oncogene. 1999; 18:6112-6120
Xin Z, Hongyan S, Jianfei C, Da S, Changlong L, Li F., EF1A interacting with nucleocapsid protein of transmissible gastroenteritis coronavirus and plays a role in virus replication. Vet Microbiol. 2014, 27; 172(3-4):443-8
Xu, Jiabao & Zhao, Shizhe & Teng, Tieshan & Abdalla, Abualgasim & Zhu, Wan & Xie, Longxiang & Wang, Yunlong & Guo, Xiangqian, Systematic Comparison of Two Animal-to-Human Transmitted Human Coronaviruses: SARS-CoV-2 and SARS-CoV. Viruses. 2020, 12. 244.
Zhou, B., Liu, J., Wang, Q., Liu, X., Li, X., Li, P., Ma, Q., Cao, C. The nucleocapsid protein of severe acute respiratory syndrome coronavirus inhibits cell cytokinesis and proliferation by interacting with translation elongation factor 1 alpha. J. Virol. 2008, 82, 6962-6971.
1. Ranieri V M, Rubenfeld G D, Thompson B T, et al. Acute respiratory distress syndrome: the Berlin Definition. JAMA 2012; 307: 2526-2533;
2. Technical document. Clinical management of COVID-19: hospital care. Date: Mar. 19, 2020 Source: Ministry of Health. Spain.
3. Wiersinga W J, Bonten M J, Boersma W G, et al. Management of community-acquired pneumonia in adults: 2016 guideline update from the Dutch Working Party on Antibiotic Policy (SWAB) and Dutch Association of Chest Physicians (NVALT). Neth J Med. 2018 January; 76 (1): 4-13.
4. Lim WS1, Baudouin S V, George R C, et al. BTS guidelines for the management of community acquired pneumonia in adults: update 2009. Thorax. 2009 October; 64 Suppl 3: iii1-55. doi: 10.1136/thx.2009.121434.]
5. Rodriguez-Morales A J, Cardona-Ospina J A, Gutiérrez-Ocampo E, et al. Clinical, laboratory and imaging features of COVID-19: A systematic review and meta-analysis. Travel Med Infect Dis. 2020 Mar. 13: 101623
6. Contingency plan for intensive medicine services against the pandemic COVID-19 of the Spanish Society of Intensive Nursing and Coronary Units.
7. Fleming T R. One-sample multiple testing procedure for phase II clinical trials. Biometrics. 1982 March; 38 (1): 143-51.
8. Rinehart K L Jr, Gloer J B, Hughes R G et al. Didemnins: antiviral and antitumor depsipeptides from a caribbean tunicate. Science. 1981 May 22; 212 (4497): 933-5.
9. Canonico P G, Pannier W L, Huggins J W, Rienehart K L. Inhibition of RNA viruses in vitro and in Rift Valley fever-infected mice by didemnins A and B. Antimicrob Agents Chemother. 1982 October; 22 (4): 696-7.
10. Losada, A., et al., Translation Elongation Factor eEF1A2 is a Novel Anticancer Target for the Marine Natural Product Plitidepsin. Sci Rep, 2016. 6: p. 35100.
11. Mateyak, M. K. and T. G. Kinzy, eEF1A: thinking outside the ribosome. J Biol Chem, 2010. 285 (28): p. 21209-13.
12. Zhou, B., et al., The nucleocapsid protein of severe acute respiratory syndrome coronavirus inhibits cell cytokinesis and proliferation by interacting with translation elongation factor Ialpha. J Virol, 2008. 82 (14): p. 6962-71.
13. Ma-Lauer, Y., et al., Influences of cyclosporin A and non-immunosuppressive derivatives on cellular cyclophilins and viral nucleocapsid protein during human coronavirus 229E replication. Antiviral Res, 2020. 173: p. 104620.
14. Cervantes-Barragan, L., et al., Dendritic cell-specific antigen delivery by coronavirus vaccine vectors induces long-lasting protective antiviral and antitumor immunity. mBio, 2010. 1(4).
15. Wang M, Cao R, Zhang L, et al. Remdesivir and chloroquine effectively inhibit the recently emerged novel coronavirus (2019-nCoV) in vitro. Cell Research 2020; 30: 269-271.
16. PharmaMar, data on file; VPT1992/2014.
17. PharmaMar, data on file; VPT2678/2014.
18. Maroun, J. A., et al., Phase I study of Aplidine in a daily×5 one-hour infusion every 3 weeks in patients with solid tumors refractory to standard therapy. A National Cancer Institute of Canada Clinical Trials Group study: NCIC CTG IND 115. Ann Oncol, 2006. 17 (9): p. 1371-8.
19. Nalda-Molina R, et al. Population pharmacokinetics meta-analysis of plitidepsin (Aplidin) in cancer subjects. Cancer Chemother Pharmacol. 2009 June; 64 (1): 97-108. doi: 10.1007/s00280-008-0841-4.

1. A compound of general formula I 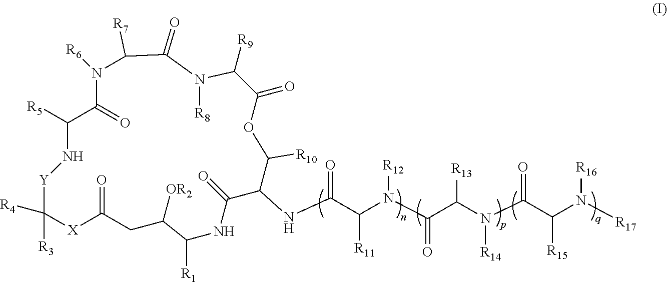
wherein X is selected from 0 and NH;
Y is selected from CO and —COCH(CH₃)CO—;
each n and p is independently selected from 0 and 1, and q is selected from 0, 1 and 2;
each R₁, R₃, R₅, R₉, R₁₁, and R₁₅ is independently selected from hydrogen, substituted or unsubstituted C₁-C₆ alkyl, substituted or unsubstituted C₂-C₆ alkenyl, and substituted or unsubstituted C₂-C₆ alkynyl;
R₂ is selected from hydrogen, CORₐ, COORₐ, substituted or unsubstituted C₁-C₆ alkyl, substituted or unsubstituted C₂-C₆ alkenyl, and substituted or unsubstituted C₂-C₆ alkynyl;
each R₄, R₈, R₁₀, R₁₂, and R₁₆ is independently selected from hydrogen and substituted or unsubstituted C₁-C₆ alkyl;
each R₇ and R₁₃ is independently selected from hydrogen, substituted or unsubstituted C₁-C₆ alkyl, substituted or unsubstituted C₂-C₆ alkenyl, and substituted or unsubstituted C₂-C₆ alkynyl; each R₆ and R₁₄ is independently selected from hydrogen and substituted or unsubstituted C₁-C₆ alkyl;
or R₆ and R₇ and/or R₁₃ and R₁₄ together with the corresponding N atom and C atom to which they are attached may form a substituted or unsubstituted heterocyclic group;
R₁₇ is selected from hydrogen, CORₐ, COORₐ, CONHR_{b}, COSR_{c}, (C═NR_{b})ORₐ, (C═NR_{b})NHR_{b}, (C═NRb)SR_{c}, (C═S)ORₐ, (C═S)NHR_{b}, (C═S)SR_{c}, SO₂R_{c}, SO₃R_{c}, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, with the proviso that when n, p, and q are 0 then R₁₇ is not hydrogen; and
each Rₐ, R_{b}, and R_{c} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
or a pharmaceutically acceptable salt or stereoisomer thereof, for use in the treatment of coronavirus (CoV) infection.

2. A compound according to claim 1, wherein R₃ and R₄ are independently selected from hydrogen and substituted or unsubstituted C₁-C₆ alkyl; preferably wherein R₃ is isopropyl and R₄ is hydrogen.

3. A compound according to claim 1 or claim 2 of general formula II, wherein R₃ and R₄ are methyl.

4. A compound according to any preceding claim, wherein R₁₁ is selected from hydrogen and substituted or unsubstituted C₁-C₆ alkyl; preferably wherein R₁₁ is methyl or isobutyl.

5. A compound according to any preceding claim, of general formula III wherein R₁₁ is methyl and n=1.

6. A compound according to any preceding claim, wherein R₁, R₅, R₉, and R₁₅ are independently selected from hydrogen and substituted or unsubstituted C₁-C₆ alkyl; preferably wherein R, is selected from sec-butyl and isopropyl, R₅ is isobutyl, R₉ is p-methoxybenzyl, and R₁₅ is selected from methyl and benzyl.

7. A compound according to any preceding claim, wherein R₈, R₁₀, R₁₂, and R₁₆ are independently selected from hydrogen and substituted or unsubstituted C₁-C₆ alkyl; preferably wherein R₈, R₁₀ and R₁₂ are methyl, and R₁₆ is hydrogen.

8. A compound according to any preceding claim, wherein R₆ and R₁₄ are independently selected from hydrogen and substituted or unsubstituted C₁-C₆ alkyl; preferably wherein R₆ is selected from hydrogen and methyl, and R₁₄ is hydrogen.

9. A compound according to any preceding claim, wherein R₇ and R₁₃ are independently selected from hydrogen and substituted or unsubstituted C₁-C₆ alkyl; preferably wherein R₇ is methyl and R₁₃ is selected from hydrogen, methyl, isopropyl, isobutyl, and 3-amino-3-oxopropyl.

10. A compound according to any of claims 1 to 7, wherein R₆ and R₇ and/or R₁₃ and R₁₄ together with the corresponding N atom and C atom to which they are attached form a substituted or unsubstituted pyrrolidine group.

11. A compound according to any preceding claim, wherein R₂ is selected from hydrogen, substituted or unsubstituted C₁-C₆ alkyl, and CORₐ, and wherein Rₐ is a substituted or unsubstituted C₁-C₆ alkyl; preferably wherein R₂ is hydrogen.

12. A compound according to any preceding claim, wherein R₁₇ is selected from hydrogen, CORₐ, COORₐ, CONHR_{b}, (C═S)NHR_{b}, and SO₂R_{c}, and wherein each Rₐ, R_{b}, and R_{c} is independently selected from substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group; preferably wherein R₁₇ is selected from hydrogen, COObenzyl, CObenzo[b]thiophen-2-yl, SO₂(p-methylphenyl), COCOCH₃ and COOC(CH₃)3.

13. A compound according to any preceding claim, wherein X is NH.

14. A compound according to any of claims 1 to 12, wherein X is O.

15. A compound according to any preceding claim wherein Y is CO.

16. A compound according to any of claims 1 to 14, wherein Y is —COCH(CH₃)CO—.

17. A compound according to claim 1 having the following structure: 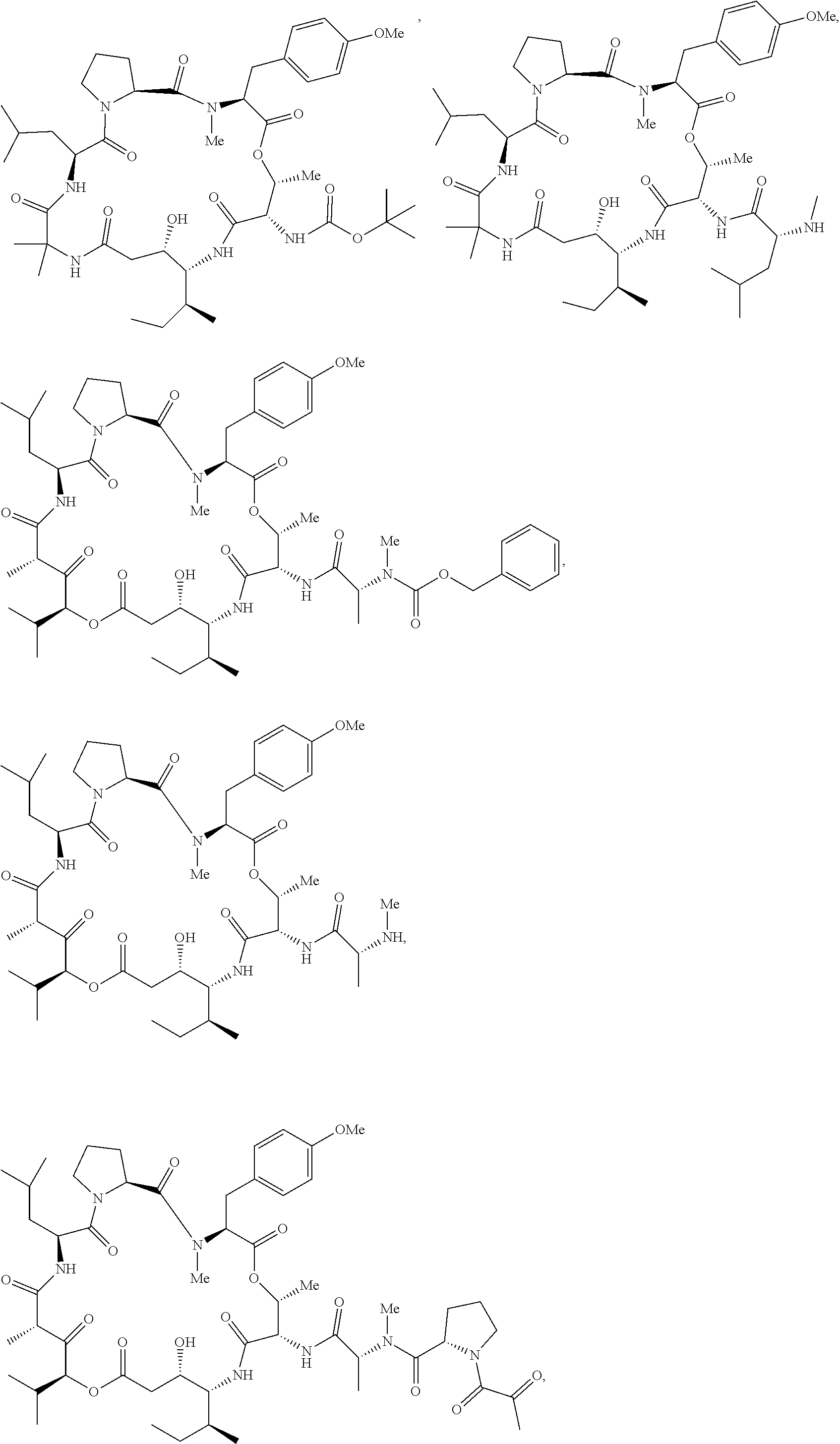 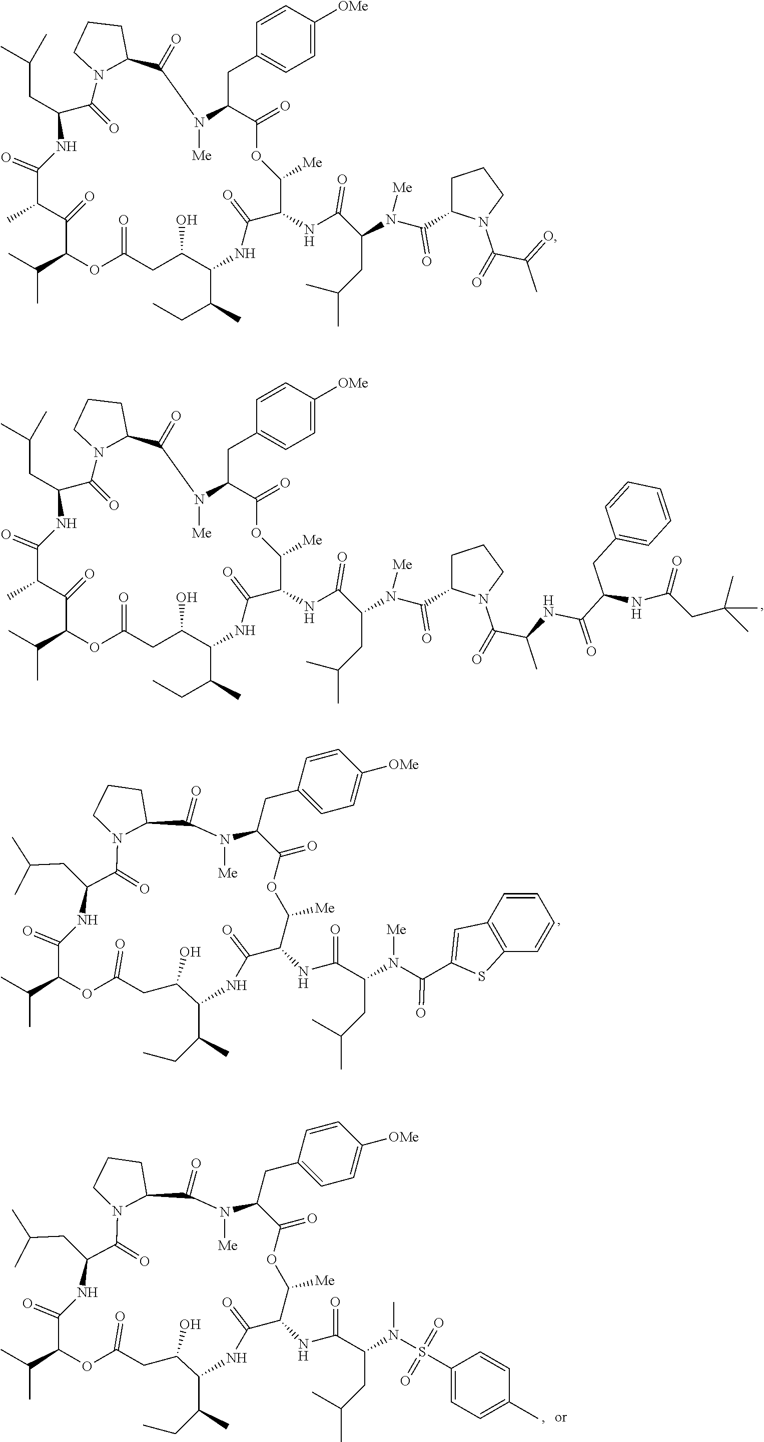 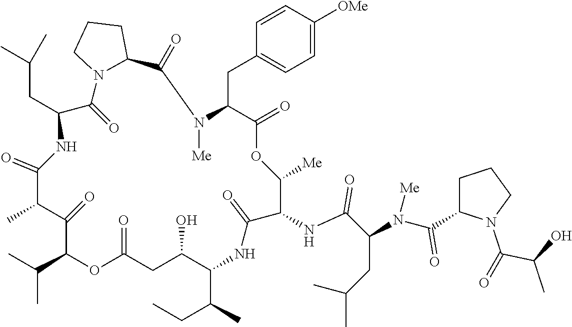 [SMILES: CC[C@H](C)[C@H]1NC(=O)[C@@H](NC(=O)[C@H](CC(C)C)N(C)C(=O)[C@@H]2CCCN2C(=O)[C@H](C)O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O]
or pharmaceutically acceptable salts or stereoisomers thereof.

18. A compound according to claim 1, wherein the compound is PLD, or pharmaceutically acceptable salts or stereoisomers thereof.

19. A compound according to claim 1, wherein the compound is didemninB, or pharmaceutically acceptable salts or stereoisomers thereof.

20. A compound according to any of the preceding claims, for use in the treatment of CoV infection, wherein the CoV is SARS-CoV-2.

21. A compound according to any one of claims 1 to 20, for use in the treatment of COVID-19 and/or for use in the treatment of pneumonia caused by COVID-19.

22. A compound according to any preceding claim, wherein the CoV infection is mild infection; and/or wherein the CoV infection is moderate infection; and/or wherein the CoV infection is severe infection.

23. A compound according to any preceding claim, wherein the CoV infection is acute CoV infection, preferably wherein the CoV infection is acute COVID-19 infection; and/or wherein the CoV infection is ongoing symptomatic CoV infection, preferably wherein the CoV infection is ongoing symptomatic COVID-19 infection; and/or wherein the CoV infection is post-CoV syndrome, CoV persistent or long CoV; preferably wherein the CoV infection is post-COVID-19 syndrome, COVID persistent or long COVID.

24. A compound according to claim 23, wherein the post-CoV syndrome, CoV persistent or long CoV include one or more symptoms arising from the cardiovascular, respiratory, gastrointestinal, neurological, musculoskeletal, metabolic, renal, dermatological, otolaryngological, haematological and autonomic systems; psychiatric problems, generalised pain, fatigue and/or persisting fever.

25. A compound according to any preceding claim, for use in the treatment of a patient with signs and symptoms of CoV infection (preferably COVID-19) for up to 4 weeks; and/or for use in the treatment of a patient with signs and symptoms of CoV infection (preferably COVID-19) from 4 weeks to 12 weeks; and/or for use in the treatment of a patient with signs and symptoms of CoV infection, preferably COVID-19, for more than 12 weeks.

26. A compound according to any preceding claim, for use in the prophylaxis, reduction or treatment of COVID persistent, long COVID or post-COVID syndrome; preferably wherein the prophylaxis, reduction or treatment minimises the likelihood that a patient suffers from COVID persistent, long COVID or post-COVID syndrome symptoms; and/or reduces the severity of such symptoms; further preferably wherein the treatment minimising the symptoms of CoV infection.

27. A compound according to any preceding claim, wherein the treatment reduces the infectivity of CoV patients; including wherein the patient is asymptomatic or not very symptomatic patients yet has a high viral load.

28. A compound for use according to any preceding claim, wherein the compound is administered in combination with a corticosteroid, preferably dexamethasone.

29. A compound for use according to claim 28, wherein the compound and corticosteroid are administered concurrently, separately or sequentially.

30. A compound for use according to any one of claims 1 to 29, wherein the compound is administered according to a regimen of a daily dose for 10 days, 9 days, 8 days, 7 days, 6 days, 5 days, 4 days, 3 days, 2 days or 1 day; preferably 2-5 days, 3-5 days, or 3, 4 or 5 days; most preferably 3 days or 5 days; most preferably 3 days.

31. A compound for use according to any one of claims 1 to 30, wherein the compound is administered at a dose of 5 mg a day or less, 4.5 mg a day or less, 4 mg a day or less, 3.5 mg a day or less, 3 mg a day or less, 2.5 mg a day or less or 2 mg a day or less; 0.5 mg/day, 1 mg/day, 1.5 mg/day, 2 mg/day, 2.5 mg/day, 3 mg/day, 3.5 mg/day, 4 mg/day, 4.5 mg/day, or 5 mg/day; preferably 1 mg/day, 1.5 mg/day, 2 mg/day or 2.5 mg/day; more preferably 1.5-2.5 mg/day; most preferably 1.5 mg/day, 2.0 mg/day or 2.5 mg/day.

32. A compound for use according to any one of claims 1 to 31, wherein the compound is administered at a total dose of 1-50 mg, 1-40 mg, 1-30 mg, 1-20 mg, 1-15 mg, 3-15 mg, 3-12 mg, 4-12 mg, 4-10 mg, or 4.5-10 mg; 4 mg, 4.5 mg, 5 mg, 5.5 mg, 6 mg, 6.5 mg, 7 mg, 7.5 mg, 8 mg, 8.5 mg, 9 mg, 9.5 mg or 10 mg; preferably 4.5 mg, 5 mg, 6 mg, 7.5 mg, 8 mg, 9 mg or 10 mg; more preferably 4.5-7.5 mg/day.

33. A compound for use according to any one of claims 1 to 32, wherein the compound is administered by infusion.

34. A compound for use according to claim 33, wherein the infusion is a 1 hour infusion, a 1.5 hour infusion, a 2 hour infusion or a 3 hour infusion, preferably a 1.5 hour infusion.

35. A compound for use according to any one of claims 1 to 34, wherein 1.5 mg of plitidepsin is administered as a 1.5-hour infusion, once a day for 3 consecutive days; or wherein 2 mg of plitidepsin is administered as a 1.5-hour infusion, once a day for 3 consecutive days; or wherein 2.5 mg of plitidepsin is administered as a 1.5-hour infusion, once a day for 3 consecutive days; or wherein 1 mg of plitidepsin is administered as a 1.5-hour infusion, once a day for 5 consecutive days; or wherein 2 mg of plitidepsin is administered as a 1.5-hour infusion, once a day for 5 consecutive days.

36. A compound for use according to any one of claims 1 to 35, wherein the compound is administered using a loading dose and a maintenance dose.

37. A compound for use according to claim 36, wherein the dosage regimen is:
a loading dose of 2.5 mg for day 1, and followed by a maintenance dose of 2 mg/day for subsequent days;
a loading dose of 2.5 mg for day 1, and followed by a maintenance dose of 1.5 mg/day for subsequent days;
a loading dose of 2.5 mg for day 1, and followed by a maintenance dose of 1 mg/day for subsequent days;
a loading dose of 2.5 mg for day 1, and followed by a maintenance dose of 0.5 mg/day for subsequent days;
a loading dose of 2 mg for day 1, and followed by a maintenance dose of 1.5 mg/day for subsequent days;
a loading dose of 2 mg for day 1, and followed by a maintenance dose of 1 mg/day for subsequent days;
a loading dose of 2 mg for day 1, and followed by a maintenance dose of 0.5 mg/day for subsequent days;
a loading dose of 1.5 mg for day 1, and followed by a maintenance dose of 1 mg/day for subsequent days;
a loading dose of 1.5 mg for day 1, and followed by a maintenance dose of 0.5 mg/day for subsequent days; or
a loading dose of 1 mg for day 1, and followed by a maintenance dose of 0.5 mg/day for subsequent days.

38. A compound for use according to any one of claims 1 to 37, wherein the compound is administered in combination with a corticosteroid, and wherein the corticosteroid is administered on the same days as administration of the compound according to any one of claims 1 to 19.

39. A compound for use according to claim 38, wherein the corticosteroid may also be administered on one or more subsequent days; preferably wherein the corticosteroid is administered with the compound on days 1-3 and the corticosteroid is further administered on one or more of days 4-10.

40. A compound for use according to claim 39, wherein the corticosteroid is administered intravenously on days when the compound is administered but is administered by oral administration or IV on subsequent days.

41. A compound for use according to any one of claims 38 to 40, wherein the corticosteroid is dexamethasone; preferably wherein dexamethasone is administered at a dose of 6.6 mg/day IV on days when the compound according to the present invention is administered.

42. A compound for use according to claim 41, wherein dexamethasone is administered at a dose of 6 mg/day oral administration or IV on subsequent days, preferably one or more of days 4, 5, 6, 7, 8, 9 and 10.

43. A compound for use according to any one of claims 1 to 42, wherein PLD is administered 1.5 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution); or
wherein PLD is administered 2.0 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution); or
wherein PLD is administered 2.5 mg/day intravenous (IV) combined with dexamethasone 6.6 mg/day IV on Days 1 to 3, followed by dexamethasone 6 mg/day oral administration (PO)/IV from Day 4 and up to Day 10 (as per physician judgement according to patient clinical condition and evolution).

44. A compound for use according to any one of claims 38 to 43, wherein the corticosteroid is administered 20 to 30 minutes prior to starting treatment with the compound according to any one of claims 1 to 19.

45. A compound for use according to any one of claims 1 to 44, wherein the patient additionally receives the following medications, preferably 20 to 30 minutes prior to starting treatment with the compound according to any one of claims 1 to 19:
Ondansetron 8 mg IV (or equivalent);
Diphenhydramine hydrochloride 25 mg IV (or equivalent); and
Ranitidine 50 mg IV (or equivalent).

46. A compound for use according to any one of claims 1 to 45, wherein on Days 4 and 5, patients receive ondansetron (or equivalent) 4 mg twice a day PO.

47. A compound for use according to any one of claims 1 to 34, wherein the compound is administered as a single dose (on day 1).

48. A compound for use according to claim 47, wherein the single dose is 1-10 mg, 4-10 mg, 4.5-10 mg, 4 mg, 4.5 mg, 5 mg, 5.5 mg, 6 mg, 6.5 mg, 7 mg, 7.5 mg, 8 mg, 8.5 mg, 9 mg, 9.5 mg or 10 mg, preferably 4.5 mg, 5 mg, 6 mg, 7.5 mg, 8 mg, 9 mg or 10 mg, more preferably 5-9 mg, 6.5-8.5 mg, 7-8 mg or most preferably 7.5 mg.

49. A compound for use according to any one of claims 47 to 48, wherein the compound is administered as a 1.5-hour infusion.

50. A compound for use according to any one of claims 47 to 49, wherein a corticosteroid is administered according to the regimen according to any one of claims 38 to 44.

51. A compound for use according to any one of claims 47 to 50, wherein the following prophylactic medications are administered 20-30 minutes prior to administration with a compound of the present invention:
Ondansetron 8 mg IV (or equivalent), particularly in slow infusion of 15 minutes;
Diphenhydramine hydrochloride 25 mg IV (or equivalent);
Ranitidine 50 mg IV (or equivalent).

52. A compound for use according to any one of claims 47 to 51, wherein ondansetron 4 mg orally is given every 12 hours for 3 days after administration of a compound of the present invention.

53. A compound for use according to any preceding claim, wherein dexamethasone is dexamethasone phosphate and is administered at a dose of 8 mg if administered on days when a compound of the invention is administered (equating to a dose of 6.6 mg base) and is administered at a dose of 7.2 mg if administered thereafter (equating to a dose of 6 mg base).

54. A pharmaceutical composition comprising a compound as defined in any of claims 1 to 53, or a pharmaceutically acceptable salt or stereoisomer thereof, and a pharmaceutically acceptable carrier, for use in the treatment of coronavirus (CoV) infection; preferably wherein the CoV is SARS-CoV-2.

55. Use of a compound as defined in any of claims 1 to 53, or a pharmaceutically acceptable salt or stereoisomer thereof, in the manufacture of a medicament for the treatment of CoV infection; preferably wherein the CoV is SARS-CoV-2.

56. A method of treating a CoV infection, wherein the method comprises administering to an individual in need thereof, a therapeutically effective amount of a compound as defined in any of claims 1 to 53, or a pharmaceutically acceptable salt or stereoisomer thereof; preferably wherein the CoV is SARS-CoV-2.

57. A method of prophylaxis, reduction or treatment of CoV persistent, long CoV or post-CoV syndrome, wherein the method comprises administering to an individual in need thereof, a therapeutically effective amount of a compound as defined in any of claims 1 to 53, or a pharmaceutically acceptable salt or stereoisomer thereof; preferably wherein the CoV is SARS-CoV-2; optionally wherein the treatment minimises the likelihood that a patient suffers from COVID persistent, long COVID or post-COVID syndrome symptoms; and/or reduces the severity of such symptoms.

58. A method of reducing the infectivity of CoV patients, wherein the method comprises administering to an individual in need thereof, a therapeutically effective amount of a compound as defined in any of claims 1 to 53, or a pharmaceutically acceptable salt or stereoisomer thereof; preferably wherein the CoV is SARS-CoV-2; optionally wherein the patient is asymptomatic or not very symptomatic yet has a high viral load.

59. A method of minimising the symptoms of CoV infection, wherein the method comprises administering to an individual in need thereof, a therapeutically effective amount of a compound as defined in any of claims 1 to 53, or a pharmaceutically acceptable salt or stereoisomer thereof; preferably wherein the CoV is SARS-CoV-2.

60. A corticosteroid for use in the treatment of CoV infection, wherein the corticosteroid is administered in combination with a compound according to any one of claims 1 to 53.

61. A compound according to any one of claims 1 to 19 and a corticosteroid for use in the treatment of CoV infection; wherein the use is according to any one of claims 1 to 53.

62. A method of treatment of CoV infection, the method comprising administering a combination therapy of compound according to any one of claims 1 to 19 or a pharmaceutically acceptable salt thereof and a corticosteroid to a patient in need thereof, thereby treating the CoV infection; wherein said method is as defined in any one of claims 1 to 53.

63. Use of a compound as defined in any of claims 1 to 53 or a pharmaceutically acceptable salt or stereoisomer thereof, in the manufacture of a medicament for the treatment of CoV infection; wherein said treatment includes administration of a corticosteroid.

64. Use of a corticosteroid in the manufacture of a medicament for the treatment of CoV infection; wherein said treatment includes administration of a compound as defined in any of claims 1 to 53 or a pharmaceutically acceptable salt or stereoisomer thereof.

65. Use of a compound as defined in any of claims 1 to 53 or a pharmaceutically acceptable salt or stereoisomer thereof and a corticosteroid in the manufacture of a medicament for the treatment of CoV infection.

66. A pharmaceutical package comprising a compound as defined in any of claims 1 to 53 and a corticosteroid, optionally further comprising instructions according to any one of claims 1 to 53.

67. A kit comprising the compound as defined in any of claims 1 to 53 together with instructions for treating CoV infections.

68. A kit comprising a compound as defined in any of claims 1 to 53 and a corticosteroid together with instructions for treating CoV infections.
